(19) United States
(12) Patent Application Publication
(10) Pub. No.: US 2024/0092914 A1
(43) Pub. Date: Mar. 21, 2024

(54) DEPLETION AND INHIBITION OF PLASMACYTOID DENDRITIC CELLS

(71) Applicant: New Paradigm Biosciences USA, Inc., Acton, MA (US)

(72) Inventor: Qi Jiang, Acton, MA (US)

(21) Appl. No.: 18/365,722

(22) Filed: Aug. 4, 2023

Related U.S. Application Data
(60) Provisional application No. 63/395,003, filed on Aug. 4, 2022.

(51) Int. Cl. C07K 16/28; A61P 35/00

(52) U.S. Cl. CPC C07K 16/2851; A61P 35/00; C07K 2317/24; C07K 2317/56; C07K 2317/92

(57) ABSTRACT

An antibody or fragment thereof, compositions, and methods of use related to depletion, inhibition, or depletion and inhibition of plasmacytoid dendritic cells (pDC), are disclosed.

### CROSS-REFERENCE TO RELATED APPLICATIONS

This application claims priority to U.S. Provisional Patent Application Ser. No. 63/395,003 filed on Aug. 4, 2022, the contents of which are incorporated herein by reference in their entirety.

### STATEMENT REGARDING SEQUENCE LISTING

This application incorporates by reference the material in the ST.26 XML file titled NPBUS-2_ST.26.xml, which was created on Aug. 4, 2022 and is 20 KB.

### FIELD

The present disclosure provides novel antibodies and compositions and uses thereof.

### BACKGROUND

Plasmacytoid dendritic cells (pDC) have been implicated in several disorders. U.S. Pat. No. 9,670,283 discloses compositions that may help deplete pDC in a patient in need thereof.

### SUMMARY

The present disclosure provides an antibody or fragment thereof comprising a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments, the antibody or fragment thereof binds an epitope having SEQ ID NO: 11 with an affinity (K_{d}) from 0.1 nanomolar (nM) to 100 nM.

In embodiments, the antibody or fragment thereof is a monoclonal antibody or fragment thereof.

In embodiments, the antibody or fragment thereof is a humanized antibody or fragment thereof.

In embodiments, the heavy chain variable region comprises an amino acid sequence given by SEQ ID NO: 1 or a sequence at least about 90% identical to SEQ ID NO: 1; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments, the heavy chain variable region comprises an amino acid sequence given by SEQ ID NO: 2 or a sequence at least about 90% identical to SEQ ID NO: 2; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments, the heavy chain variable region comprises an amino acid sequence given by SEQ ID NO: 3 or a sequence at least about 90% identical to SEQ ID NO: 3; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments, the heavy chain variable region comprises an amino acid sequence given by SEQ ID NO: 4 or a sequence at least about 90% identical to SEQ ID NO: 4; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 5 or a sequence at least about 90% identical to SEQ ID NO: 5.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 6 or a sequence at least about 90% identical to SEQ ID NO: 6.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 7 or a sequence at least about 90% identical to SEQ ID NO: 7.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 8 or a sequence at least about 90% identical to SEQ ID NO: 8.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 9 or a sequence at least about 90% identical to SEQ ID NO: 9.

In embodiments, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and the light chain variable region comprises an amino acid sequence given by SEQ ID NO: 10 or a sequence at least about 90% identical to SEQ ID NO: 10.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 5 or a sequence at least about 90% identical to SEQ ID NO: 5.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 5 or a sequence at least about 90% identical to SEQ ID NO: 5.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 5 or a sequence at least about 90% identical to SEQ ID NO: 5.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 5 or a sequence at least about 90% identical to SEQ ID NO: 5.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 6 or a sequence at least about 90% identical to SEQ ID NO: 6.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 7 or a sequence at least about 90% identical to SEQ ID NO: 7.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 8 or a sequence at least about 90% identical to SEQ ID NO: 8.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 9 or a sequence at least about 90% identical to SEQ ID NO: 9.

In embodiments, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 10 or a sequence at least about 90% identical to SEQ ID NO: 10.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 6 or a sequence at least about 90% identical to SEQ ID NO: 6.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 6 or a sequence at least about 90% identical to SEQ ID NO: 6.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 6 or a sequence at least about 90% identical to SEQ ID NO: 6.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 7 or a sequence at least about 90% identical to SEQ ID NO: 7.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 7 or a sequence at least about 90% identical to SEQ ID NO: 7.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 7 or a sequence at least about 90% identical to SEQ ID NO: 7.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 8 or a sequence at least about 90% identical to SEQ ID NO: 8.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 8 or a sequence at least about 90% identical to SEQ ID NO: 8.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 8 or a sequence at least about 90% identical to SEQ ID NO: 8.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 9 or a sequence at least about 90% identical to SEQ ID NO: 9.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 9 or a sequence at least about 90% identical to SEQ ID NO: 9.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 9 or a sequence at least about 90% identical to SEQ ID NO: 9.

In embodiments, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 10 or a sequence at least about 90% identical to SEQ ID NO: 10.

In embodiments, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 10 or a sequence at least about 90% identical to SEQ ID NO: 10.

In embodiments, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 10 or a sequence at least about 90% identical to SEQ ID NO: 10.

In embodiments, a method for depleting, inhibiting, or depleting and inhibiting plasmacytoid dendritic cells (pDC) in a patient in need thereof includes administering to the patient an effective amount of an antibody or fragment thereof that specifically binds to an epitope comprising blood dendritic cell antigen-2 (BDCA2) and depletes, inhibits, or depletes and inhibits pDC, thereby depleting, inhibiting, or depleting and inhibiting pDC. The antibody or fragment thereof comprises: a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and a light chain variable region comprising an amino acid sequence selected from the group consisting of SEQ ID NO: 5, SEQ ID NO:6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for depleting, inhibiting, or depleting and inhibiting pDC, the epitope comprises an amino acid sequence given by SEQ ID NO: 11, and the antibody or fragment thereof binds the epitope with an affinity (K_{d}) from 0.1 nanomolar (nM) to 100 nM.

In embodiments of the method for depleting, inhibiting, or depleting and inhibiting pDC, the antibody or fragment thereof is a monoclonal antibody or fragment thereof.

In embodiments of the method for depleting, inhibiting, or depleting and inhibiting pDC, the antibody or fragment thereof is a humanized antibody or fragment thereof.

In embodiments of the method for depleting, inhibiting, or depleting and inhibiting pDC, the BDCA2 is human BDCA2.

In embodiments, a method for treating a disorder associated with plasmacytoid dendritic cells (pDC) in a patient in need thereof includes administering to the patient an effective amount of an antibody or a fragment thereof that specifically binds to an epitope comprising blood dendritic cell antigen-2 (BDCA2) and depletes, inhibits, or depletes and inhibits pDC, thereby treating the disorder. The antibody or fragment thereof comprises: a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for treating a disorder associated with pDC, the disorder is selected from the group consisting of an infectious disease, an autoimmune disease, an inflammatory condition, a respiratory condition, a skin disorder, a cancer, and a combination of two or more of these.

In embodiments of the method for treating a disorder associated with pDC, the disorder is an infectious disease associated with infection by an infectious agent.

In embodiments of the method for treating a disorder associated with pDC, the infectious agent is one or more viruses or one or more microorganisms.

In embodiments of the method for treating a disorder associated with pDC, the infectious agent is a virus selected from the group consisting of Hepadnaviridae; Flaviviridae; Retroviridae; Herpesviridae; Papovaviridae; Rhabdoviridae; Paramyxoviridae; Reoviridae; Bunyaviridae; Filoviridae; Adenoviridae; Parvoviridae; Arenaviridae; Orthomyxoviridae; Poxviridae; Togaviridae; Coronaviridae; Picornaviridae; rhinoviruses; orbiviruses; picodnaviruses; encephalomyocarditis virus (EMV); Parainfluenza viruses; adenoviruses; Coxsackieviruses; Echoviruses; Rubeola virus; Rubella virus; human papillomaviruses; Canine distemper virus; Canine contagious hepatitis virus; Feline calicivirus; Feline rhinotracheitis virus; TGE virus (swine); Foot and mouth disease virus; simian virus 5; human parainfluenza virus type 2; human metapneuomovirus; and enteroviruses.

In embodiments of the method for treating a disorder associated with pDC, the infectious agent is a microorganism selected from the group consisting of Rickettsia, Chlamydia, Mycobacteria, Clostridia, Corynebacteria, Mycoplasma, Ureaplasma, Legionella, Shi-gella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., and Helicobacter spp.

In embodiments of the method for treating a disorder associated with pDC, the infectious agent is a microorganism selected from the group consisting of Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum. Bacillus anthracis, Salmonella typhi, Vibrio cholera. Pasteurella pestis (Yersinia pestis), Pseudomonas aeruginosa, Campylobacter jejuni, Clostridium dificile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

In embodiments of the method for treating a disorder associated with pDC, the disorder comprises an autoimmune disease selected from the group consisting of diabetes, cutaneous psoriasis, hyperthyroidism, autoimmune adrenal insufficiency, hemolytic anemia, rheumatic carditis, systemic lupus erythematosus, cutaneous lupus erythematosis, psoriatic arthritis, Sjogren's syndrome polymyositis, myositis, multiple sclerosis, Crohn's disease, ulcerative colitis, lupus, inflammatory bowel syndrome, dermatomyositis, dermatitis, atopic dermatitis, scleroderma, polymyositis, psoriasis, alopecia areata, rheumatoid arthritis, graft-versus-host disease, interferonopathies, irritable bowel syndrome, and a combination of two or more of these.

In embodiments of the method for treating a disorder associated with pDC, the disorder comprises an inflammatory condition, the inflammatory condition comprising acute kidney injury.

In embodiments of the method for treating a disorder associated with pDC, the disorder comprises a respiratory condition, the respiratory condition comprising asthma, lung fibrosis, or a combination of asthma and lung fibrosis.

In embodiments of the method for treating a disorder associated with pDC, the disorder comprises a skin disorder selected from the group consisting of scleroderma, psoriasis, atopic dermatitis, dermamyosititis, and a combination of two or more of these.

In embodiments of the method for treating a disorder associated with pDC, the disorder comprises a cancer selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, B-cell acute lymphocytic leukemia, blastic plasmacytoid dendritic cell neoplasm (“BPDCN” or “pDC leukemia”), chronic lymphocytic leukemia, acute myelogenous leukemia, chronic myelogenous leukemia (“AML”), chronic granulocytic leukemia, acute granulocytic leukemia, aging-related hairy cell leukemia, chronic myelomonocytic leukemia (“CMML”), myelodysplastic syndrome, chronic myelofibrosis, multiple myeloma, neuroblastoma, rhabdomyosarcoma, Kaposi's sarcoma, Waldenström's macroglobulinemia, diffuse large B-cell lymphoma (DLBCL) (L265P), polycythemia vera, essential thrombocytosis, Hodgkin's disease, non-Hodgkin's lymphoma, soft-tissue sarcoma, osteogenic sarcoma, primary macroglobulinemia, retinoblastoma, and a combination of two or more of these.

In embodiments of the method for treating a disorder associated with pDC, the epitope comprises an amino acid sequence given by SEQ ID NO: 11 and the antibody or fragment thereof binds the epitope with an affinity (K_{d}) from 0.1 nanomolar (nM) to 100 nM.

In embodiments of the method for treating a disorder associated with pDC, the antibody or fragment thereof is a monoclonal antibody or fragment thereof.

In embodiments of the method for treating a disorder associated with pDC, the antibody or fragment thereof is a humanized antibody or fragment thereof.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 5 or a sequence at least about 90% identical to SEQ ID NO: 5.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 6 or a sequence at least about 90% identical to SEQ ID NO: 6.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 7 or a sequence at least about 90% identical to SEQ ID NO: 7.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 8 or a sequence at least about 90% identical to SEQ ID NO: 8.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 9 or a sequence at least about 90% identical to SEQ ID NO: 9.

In embodiments of the method for treating a disorder associated with pDC, the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO:2, SEQ ID NO: 3, or SEQ ID NO: 4; and the light chain variable region comprises an amino acid sequence given by SEQ ID NO: 10 or a sequence at least about 90% identical to SEQ ID NO: 10.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 5 or a sequence at least about 90% identical to SEQ ID NO: 5. In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 5 or a sequence at least about 90% identical to SEQ ID NO: 5.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 5 or a sequence at least about 90% identical to SEQ ID NO: 5.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 5 or a sequence at least about 90% identical to SEQ ID NO: 5.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 6 or a sequence at least about 90% identical to SEQ ID NO: 6.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 7 or a sequence at least about 90% identical to SEQ ID NO: 7.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 8 or a sequence at least about 90% identical to SEQ ID NO: 8.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 9 or a sequence at least about 90% identical to SEQ ID NO: 9.

In embodiments of the method for treating a disorder associated with pDC, the heavy chain variable region comprises an amino acid sequence given by SEQ ID NO: 1 or a sequence at least about 90% identical to SEQ ID NO: 1; and the light chain variable region comprises an amino acid sequence given by SEQ ID NO: 10 or a sequence at least about 90% identical to SEQ ID NO: 10.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 6 or a sequence at least about 90% identical to SEQ ID NO: 6.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 6 or a sequence at least about 90% identical to SEQ ID NO: 6.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 6 or a sequence at least about 90% identical to SEQ ID NO: 6.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 7 or a sequence at least about 90% identical to SEQ ID NO: 7.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 7 or a sequence at least about 90% identical to SEQ ID NO: 7.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 7 or a sequence at least about 90% identical to SEQ ID NO: 7.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 8 or a sequence at least about 90% identical to SEQ ID NO: 8.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 8 or a sequence at least about 90% identical to SEQ ID NO: 8.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 8 or a sequence at least about 90% identical to SEQ ID NO: 8.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 9 or a sequence at least about 90% identical to SEQ ID NO: 9.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 9 or a sequence at least about 90% identical to SEQ ID NO: 9.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 9 or a sequence at least about 90% identical to SEQ ID NO: 9.

In embodiments of the method for treating a disorder associated with pDC, the heavy chain variable region comprises an amino acid sequence given by SEQ ID NO: 2 or a sequence at least about 90% identical to SEQ ID NO: 2; and the light chain variable region comprises an amino acid sequence given by SEQ ID NO: 10 or a sequence at least about 90% identical to SEQ ID NO: 10.

In embodiments of the method for treating a disorder associated with pDC, the heavy chain variable region comprises an amino acid sequence given by SEQ ID NO: 3 or a sequence at least about 90% identical to SEQ ID NO: 3; and the light chain variable region comprises an amino acid sequence given by SEQ ID NO: 10 or a sequence at least about 90% identical to SEQ ID NO: 10.

In embodiments of the method for treating a disorder associated with pDC, the heavy chain variable region comprises an amino acid sequence given by SEQ ID NO: 4 or a sequence at least about 90% identical to SEQ ID NO: 4; and the light chain variable region comprises an amino acid sequence given by SEQ ID NO: 10 or a sequence at least about 90% identical to SEQ ID NO: 10.

In embodiments, a kit for depleting, inhibiting, or depleting and inhibiting plasmacytoid dendritic cells (pDC) in a patient in need thereof includes: instructions; and an antibody or fragment thereof. The antibody or fragment thereof includes a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and a light chain variable region comprising an amino acid sequence selected from the group consisting of SEQ ID NO: 5, SEQ ID NO: 6, SEQ ID NO: 7, SEQ ID NO:8, SEQ ID NO: 9, SEQ ID NO: 10, and a sequence at least about 90% identical to SEQ ID NO: 5, SEQ ID NO: 6, SEQ ID NO: 7, SEQ ID NO:8, SEQ ID NO: 9, or SEQ ID NO: 10.

In embodiments, a pharmaceutical composition includes: at least one excipient; and an antibody or fragment thereof. The antibody or fragment thereof includes a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and a light chain variable region comprising an amino acid sequence selected from the group consisting of SEQ ID NO: 5, SEQ ID NO: 6, SEQ ID NO: 7, SEQ ID NO:8, SEQ ID NO: 9, SEQ ID NO: 10, and a sequence at least about 90% identical to SEQ ID NO: 5, SEQ ID NO: 6, SEQ ID NO: 7, SEQ ID NO:8, SEQ ID NO: 9, or SEQ ID NO: 10.

In embodiments, an antibody or fragment thereof may be used in therapy.

In embodiments, an antibody or fragment thereof may be used for depleting, inhibiting, or depleting and inhibiting plasmacytoid dendritic cells (pDC).

In embodiments, an antibody or fragment thereof may be used for the manufacture of a medicament.

This summary is not intended to identify key or essential features of the claimed subject matter, nor is it intended to be used as an aid in limiting the scope of the claimed subject matter. Further embodiments, forms, features, and aspects of the present disclosure shall become apparent from the description and figures provided herewith.

### BRIEF DESCRIPTION OF THE DRAWINGS

The teachings of some embodiments of the present disclosure will be better understood by reference to the description taken in conjunction with the accompanying drawings, wherein:

FIG. 1 shows a schematic diagram of the expression vectors used to produce chimeric and humanized anti-BDCA2 antibodies according to embodiments;

FIG. 2 shows the SDS-PAGE analysis of NPB-303 according to embodiments;

FIG. 3 shows the protein analysis of NPB-303 on a Superose 6 column using an AKTA FPLC system according to embodiments; FIG. 3A. BioRad's Gel Filtration Standard; B. NPB-303.

FIG. 4 shows the biophysical binding of 15b, 12b, and NPB-303 to BDCA2 antigens from either monkey (Panel A) or human (Panel B) according to embodiments;

FIG. 5 shows a structural model of the BDCA2 extracellular domain according to embodiments; Green: BDCA2 extracellular domain; Yellow: NPB-303 epitope and Orange: BDCA2 lectin (ligand) binding residues.

FIG. 6 shows structural model of the NPB-303 variable (binding) domain according to embodiments; Green: VH framework; Yellow: VH CDRs; Blue: VL framework; Brown: VL CDRs and Red: Aspartic acid isomerization motifs.

FIG. 7 shows binding characterization of NPB-303 antibodies by surface plasmon resonance (SPR) according to embodiments;

FIG. 8 shows binding characterization of NPB-303 mAb to human BDCA2 antigen in vitro according to embodiments;

FIG. 9 shows binding characterization of NPB-303 mAb to human pDC in vitro according to embodiments;

FIG. 10 shows FACS plots in which NPB-303 inhibits pDC IFNα response to CpG-A stimulation in vitro according to embodiments;

FIG. 11 shows a dose-response curve for NPB-303 versus IFNα according to embodiments;

FIG. 12 shows the IC₅₀ of NPB-303 in multiple human donor PBMC samples according to embodiments;

FIG. 13 shows the potency analysis of NPB-303 in vitro according to embodiments;

FIG. 14 shows the pharmacokinetic profile of NPB-303 in genetically modified mice according to embodiments;

FIG. 15 shows FACS plots exhibiting pDC depletion and BDCA2 down-regulation by NPB-303 in the blood according to embodiments;

FIG. 16 shows summarized data exhibiting pDC depletion (% CD4+CD123+ of Lin−) and BDCA2 down-regulation (% CD4+BDCA2+ of Lin−) on pDCs according to embodiments;

FIG. 17 shows the pharmacodynamics profile of NPB-303 mAb in humanized mice in vivo according to embodiments;

FIG. 18 shows a summary of % pDCs or % BDCA2+ cells from humanized mice treated with either isotype control or NPB-303 (normalized to isotype control antibody) according to embodiments;

FIG. 19 shows summarized analysis of human BDCA2+ pDCs in splenocytes of humanized mice according to embodiments;

FIG. 20 shows summarized analysis of human CD123+ pDCs in splenocytes of humanzied mice according to embodiments;

FIG. 21 shows the summary of other cell types (Lin−CD4−, CD4+CD123− and CD4−CD123−) in the blood of humanized mice treated with isotype control mAb or NPB-303 according to embodiments;

FIG. 22 shows endotoxin of NPB-303 (1:1000 dilution) testing performed using a Kinetic Turbidimetric and Kinetic Chromogenic Testing method (ENDOSAFE);

FIG. 23 shows the summary of anti-tumor activities of NPB-303 or KEYTRUDA® in mice bearing Human Immune and Tumor cells (“HIT mice”) according to embodiments;

FIG. 24 shows the summary of body weight of tumor-bearing mice from various treatment groups according to embodiments;

FIG. 25 shows the summary of pDC depletion in spleen by NPB-303 but not KEYTRUDA® in tumor-bearing HIT mice according to embodiments;

FIG. 26 shows the summary of pDC depletion in tumor by NPB-303 but not KEYTRUDA® in tumor-bearing HIT mice according to embodiments;

FIG. 27 shows the summary of BDCA2 down-regulation in spleen by NPB-303 but not KEYTRUDA® in tumor-bearing HIT mice according to embodiments;

FIG. 28 shows the summary of BDCA2 down-regulation in tumor by NPB-303 but not KEYTRUDA® in tumor-bearing HIT mice according to embodiments;

FIG. 29 shows a schematic illustration of the protocol for the establishment of tumor-bearing humanized mouse model according to embodiments;

FIG. 30 shows representative flow plots of human pDCs in tumor isolated from the HIT-TNBC mice according to embodiments;

FIG. 31 shows a summary of percentages of human pDCs in the tumors according to embodiments;

FIG. 32 shows representative flow plots of human T cells in the tumor vs. blood isolated from HIT-TNBC mice according to embodiments;

FIG. 33 shows the mean fluorescence intensities (MFIs) of Tim3 and PD1 on CD4+ T cells according to embodiments;

FIG. 34 shows the mean fluorescence intensities (MFIs) of Tim3 and PD1 on CD8+ T cells according to embodiments;

FIG. 35 shows a dose-dependent anti-tumor activity (via tumor volume measurement) with NPB-303 according to embodiments;

FIG. 36 shows stable body weight over time for NPB-303 treated HIT mice according to embodiments;

FIG. 37 shows representative flow plots and summary of % CD4+CD123+ of Lin− in spleen according to embodiments;

FIG. 38 shows representative flow plots and summary of % CD4+CD123+ of Lin− in tumor according to embodiments;

FIG. 39 shows tumor volume over time for a second human TNBC cell line (MDA-MB-468) according to embodiments;

FIG. 40 shows a dose-dependent anti-tumor activity (via tumor volume measurement) with NPB-303 according to embodiments;

FIG. 41 shows stable body weight over time for NPB-303 treated HIT mice according to embodiments;

FIG. 42 shows a study procedure of NPB-303 and ABRAXANE® combination therapy according to embodiments;

FIG. 43 shows anti-tumor activity (via tumor volume measurement) with NPB-303 alone, ABRAXANE® alone, and a combination therapy according to embodiments;

FIG. 44 shows stable body weight over time for HIT mice treated with NPB-303 alone, ABRAXANE® alone, and a combination therapy according to embodiments;

FIG. 45 shows that PD1 was down-regulated in tumor infiltration CD8+ cells in both NPB-303 and combination therapy groups compared to the isotype control group according to embodiments;

FIG. 46 shows that Tim3 was down-regulated in tumor infiltration CD8+ cells in both NPB-303 and combination therapy groups compared to the isotype control group according to embodiments;

FIG. 47 shows representative liver images from each treatment group (isotype control group, NPB-303 alone, ABRAXANE® alone, and a combination therapy) demonstrating the reduction of tumor nodules in the groups of mice treated with NPB-303 alone or in combination with ABRAXANE® of according to embodiments;

FIG. 48 shows a summary of liver infiltration score for each treatment group (isotype control group, NPB-303 alone, ABRAXANE® alone, and a combination therapy) according to embodiments;

FIG. 49 shows representative H&E staining data of the histological sections (100 microns) of liver tissues from each treatment group (isotype control group, NPB-303 alone, ABRAXANE® alone, and a combination therapy) according to embodiments;

FIG. 50 shows representative lung H&E staining images (3× and 17×) according to embodiments;

FIG. 51 shows a summary of lung infiltration score for each treatment group (isotype control group, NPB-303 alone, ABRAXANE® alone, and a combination therapy) according to embodiments;

FIG. 52 shows thermal denaturation (Tₘ) and aggregation onset (T_{agg}) for NPB-303 compared to therapeutic antibodies that progressed at least to clinical trials according to embodiments;

FIG. 53 shows the binding activities of NPB-303 at various storage temperature conditions according to embodiments;

FIG. 54 shows a detailed sequence modification of the NPB-303 antibody according to embodiments;

FIG. 55 shows sensorgrams of SPR based affinity testing for 24 antibodies according to embodiments;

FIG. 56 shows a set of graphs of IFNα inhibition assay according to embodiments;

FIG. 57 shows a summary of the potencies (IC₅₀) of antibodies according to embodiments;

FIG. 58 shows sensorgrams of SPR based affinity testing for 3 antibodies according to embodiments;

FIG. 59 shows a set of graphs demonstrating dose dependent inhibition of TLR-9-induced IFNα by PBMC from two healthy human donors using NPB-303 and three additional monoclonal antibodies according to embodiments;

FIG. 60 shows a summary of the potencies (IC₅₀) of antibodies to IFNα inhibition according to embodiments;

FIG. 61 shows a representative set of FACS plots of human pDCs (CD4+CD123+) and CD4+BDCA2+ cells (gated on human CD45+ then Lin− cells) in blood from humanized mice treated with isotype control antibody, NPB-303, and 3 additional antibodies according to embodiments;

FIG. 62 shows a summary of % pDCs of Lin− cells in blood from humanized mice treated with isotype control antibody, NPB-303, and 3 additional antibodies according to embodiments;

FIG. 63 shows a summary of % BDCA2+ of Lin− cells in blood from humanized mice treated with isotype control antibody, NPB-303, and 3 additional antibodies according to embodiments;

FIG. 64 shows a representative set of FACS plots of human pDCs (CD4+CD123+) and CD4+BDCA2+ cells (gated on human CD45+ then Lin− cells) in spleen from humanized mice treated with isotype control antibody, NPB-303, and 3 additional antibodies according to embodiments;

FIG. 65 shows a summary of % pDCs of Lin− cells in spleen from humanized mice treated with isotype control antibody, NPB-303, and 3 additional antibodies according to embodiments;

FIG. 66 shows a summary of % BDCA2+ of Lin− cells in spleen from humanized mice treated with isotype control antibody, NPB-303, and 3 additional antibodies according to embodiments;

FIG. 67 shows a representative set of FACS plots of myeloid cells (Lin−CD4−), conventional DCs (CD4+CD123−) and other cell types (Lin−CD4−CD123−) in blood from humanized mice treated with isotype control antibody, NPB-303, and 3 additional antibodies according to embodiments;

FIG. 68 shows a summary of % Lin−CD4−, CD4+CD123−, and Lin−CD4−CD123− cells in blood from humanized mice treated with isotype control antibody, NPB-303, and 3 additional antibodies according to embodiments; and

FIG. 69 shows a summary of % Lin−CD4−, CD4+CD123−, and Lin−CD4−CD123− cells in spleen from humanized mice treated with isotype control antibody, NPB-303, and 3 additional antibodies according to embodiments.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Amino acids are represented herein in the manner recommended by the IUPAC-IUB Biochemical Nomenclature Commission, or by either the one-letter code or the three letter code, both in accordance with 37 C.F.R. § 1.822 and established usage.

The present disclosure provides an antibody or fragment thereof comprising a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

As used in the description and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”). The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The transitional phrase “consisting essentially of” means that the scope of a claim is to be interpreted to encompass the specified materials or steps recited in the claim, “and those that do not materially affect the basic and novel characteristic(s)” of the claimed subject matter. See, In re Herz, 537 F.2d 549, 551-52, 190 USPQ 461, 463 (CCPA 1976) (emphasis in the original); see also MPEP § 2111.03 (9^{th} edition, 10^{th} revision). The term “consists essentially of” (and grammatical variants), as applied to a polypeptide sequence, means a polypeptide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence such that the function of the polypeptide is not materially altered. The additional amino acids includes the total number of additional amino acids on both ends added together. The term “materially altered,” as applied to polypeptides, refers to an increase or decrease in epitope binding activity of at least about 50% or more as compared to the activity of a polypeptide consisting of the recited sequence.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence, or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “fragment,” as applied to a polypeptide, may be understood to mean an amino acid sequence of reduced length relative to a reference polypeptide or amino acid sequence and comprising, consisting essentially of, and/or consisting of an amino acid sequence of contiguous amino acids identical or almost identical (e.g., about 90%, about 92%, about 95%, about 98%, about 99% identical) to the reference polypeptide or amino acid sequence. Such a polypeptide fragment according to the disclosure may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence.

As used herein, the terms “protein” and “polypeptide” are used interchangeably and encompass both peptides and proteins, unless indicated otherwise.

As used herein, a “functional polypeptide” or “functional fragment” is one that substantially retains at least one biological activity normally associated with that polypeptide (e.g., target protein binding). In particular embodiments, the “functional polypeptide” or “functional fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional polypeptide” is one that exhibits little or essentially no detectable biological activity normally associated with the polypeptide (e.g., at most, only an insignificant amount, e.g., less than about 10% or even less than about 5%). Biological activities such as protein binding can be measured using assays known to one of ordinary skill in the art and as described herein.

Disclosed herein are antibodies that specifically bind to an epitope comprising blood dendritic cell antigen-2 (BDCA2) and deplete, inhibit, or deplete and inhibit pDC. Such antibodies may be used to deplete, inhibit, or deplete and inhibit pDC in a patient, e.g., for research or therapeutic purposes. Such antibodies can be used to treat disorders associated with pDC. Accordingly, one aspect relates to antibodies or fragments thereof that specifically bind to BDCA2 and deplete, inhibit, or deplete and inhibit pDC when administered to a patient.

The term “antibody” or “antibodies,” as used herein, refers to all types of immunoglobulins, including IgG, IgM, IgA, IgD, and IgE. The antibody may be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or 4,816,567. The antibodies can also be chemically constructed according to the method disclosed in U.S. Pat. No. 4,676,980.

Antibody fragments included, for example, Fab, Fab′, F(ab′)2, and Fv fragments; domain antibodies, diabodies; vaccibodies, linear antibodies; single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)).

Antibodies may be altered or mutated for compatibility with species other than the species in which the antibody was produced. For example, antibodies may be humanized or camelized. When the patient is a human, the antibody may be a humanized antibody. See, e.g., Safdari, et al., Antibody humanization methods—a review and update. Biotechnol. Genet. Eng. Rev., 2013; 29:175-86. Humanized forms of non-human (e.g., murine) antibodies are chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some embodiments, Fv framework residues of the human immunoglobulin are replaced by corresponding non-human residues. Humanized antibodies may also comprise residues which are found neither in the recipient antibody nor in the imported CDR or framework sequences. In general, the humanized antibody may comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody may also comprise at least a portion of an immunoglobulin constant region (Fc), typically that of a human immuno-globulin (Jones et al., Nature 321:522 (1986); Riechmann et al., Nature, 332:323 (1988); and Presta, Curr. Op. Struct. Biol. 2:593 (1992)).

As noted above, methods for humanizing non-human antibodies are known. Generally, a humanized antibody has one or more amino acid residues introduced into it from a source which is non-human. These non-human amino acid residues are often referred to as “import” residues, which are typically taken from an “import” variable domain. Humanization can essentially be performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies may be human antibodies in which some CDR residues (e.g., all of the CDRs or a portion thereof) and possibly some FR residues are substituted by residues from analogous sites in rodent (e.g., murine) antibodies.

Human antibodies may be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, Inc.: New York, p. 77 (1985) and Boerner et al., J. Immunol. 147:86 (1991)). Similarly, human antibodies may be made by introducing human immunoglobulin loci into transgenic animals, e.g., mice, in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio Technology 10:779 (1992); Lonberg et al., Nature 368:856 (1994); Morrison, Nature 368:812 (1994); Fishwild et al., Nature Biotechnol. 14:845 (1996); Neuberger, Nature Biotechnol. 14:826 (1996); and Lonberg and Huszar, Intern. Rev. Immunol. 13:65 (1995).

Polyclonal antibodies used to carry out the present disclosure can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures. The polynucleotide sequence and polypeptide sequence of BDCA2 is known and can be found in sequence databases such as GenBank. Examples of sequences include the human BDCA2 polypeptide sequence (Accession No. Q8WTTO) and polynucleotide sequence (Accession No. AF293615), incorporated herein by reference in their entirety.

Monoclonal antibodies may be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells may then be immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

In some embodiments, the antibody or fragment thereof formed by the methods described above may bind an epitope having SEQ ID NO: 11 with an affinity (K_{d}) from 0.1 nanomolar (nM) to 100 nM. SEQ ID NO: 11 is a fragment of SEQ ID NO: 12, which provides the amino acid sequence of an epitope of a BDCA2 antigen. In embodiments, the affinity (K_{d}) may be from 0.1 nM to 90 nM, from 0.1 nM to 80 nM, from 0.1 nM to 70 nM, from 0.1 nM to 60 nM, from 0.1 nM to 50 nM, from 0.1 nM to 40 nM, from 0.1 nM to 30 nM, from 0.1 nM to 20 nM, from 0.1 nM to 10 nM, from 0.1 nM to 9 nM, from 0.1 nM to 8 nM, from 0.1 nM to 7 nM, from 0.1 nM to 6 nM, from 0.1 nM to 5 nM, from 0.1 nM to 4 nM, from 0.1 nM to 3 nM, from 0.1 nM to 2 nM, from 0.1 nM to 1 nM, from 0.1 nM to 0.9 nM, from 0.1 nM to 0.8 nM, from 0.1 nM to 0.7 nM, from 0.1 nM to 0.6 nM, from 0.1 nM to 0.5 nM, from 0.1 nM to 0.4 nM, from 0.1 nM to 0.3 nM, from 0.1 nM to 0.2 nM, from 0.2 nM to 100 nM, from 0.3 nM to 100 nM, from 0.4 nM to 100 nM, from 0.5 nM to 100 nM, from 0.6 nM to 100 nM, from 0.7 nM to 100 nM, from 0.8 nM to 100 nM, from 0.9 nM to 100 nM, from 1 nM to 100 nM, from 2 nM to 100 nM, from 3 nM to 100 nM, from 4 nM to 100 nM, from 5 nM to 100 nM, from 6 nM to 100 nM, from 7 nM to 100 nM, from 8 nM to 100 nM, from 9 nM to 100 nM, from 10 nM to 100 nM, from 20 nM to 100 nM, from 30 nM to 100 nM, from 40 nM to 100 nM, from 50 nM to 100 nM, from 60 nM to 100 nM, from 70 nM to 100 nM, from 80 nM to 100 nM, or even from 90 nM to 100 nM.

The term “affinity,” as used herein, refers to the extent, or strength, of binding of the antibody to the epitope. Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay). It is understood that an affinity is an average affinity for a given population of antibodies which bind to an epitope. Various immunoassays can be used for screening to identify antibodies having the desired affinity for the peptide sequence given by SEQ ID NO: 11.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵S, ¹³¹I), enzyme labels (e.g., horseradish peroxidase, alkaline phosphatase), and fluorescence labels (e.g., fluorescein), in accordance with known techniques. Determination of the formation of an antibody/antigen complex in the methods disclosed herein can be by detection of, for example, precipitation, agglutination, flocculation, radioactivity, color development or change, fluorescence, luminescence, and the like.

In some embodiments, the CDRs of the antibody may include at least one heavy chain variable region and at least one light chain variable region.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto. In some embodiments, the antibody or fragment thereof comprises a heavy chain variable region comprising at least 50 contiguous amino acids of the amino acid sequence of SEQ ID NO: 1 or a sequence at least about 90% identical thereto, e.g., at least about 100, 150, or 200 or more contiguous amino acids.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto. In some embodiments, the antibody or fragment thereof comprises a heavy chain variable region comprising at least about 50 contiguous amino acids of the amino acid sequence of SEQ ID NO: 2 or a sequence at least about 90% identical thereto, e.g., at least about 100, 150, or 200 or more contiguous amino acids.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto. In some embodiments, the antibody or fragment thereof comprises a heavy chain variable region comprising at least about 50 contiguous amino acids of the amino acid sequence of SEQ ID NO: 3 or a sequence at least about 90% identical thereto, e.g., at least about 100, 150, or 200 or more contiguous amino acids.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto. In some embodiments, the antibody or fragment thereof comprises a heavy chain variable region comprising at least about 50 contiguous amino acids of the amino acid sequence of SEQ ID NO: 4 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 5 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 5 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 6 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 6 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 7 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 7 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 8 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 8 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 9 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 9 or a sequence at least about 90% identical thereto, e.g., at least about 100, 150, or 200 or more contiguous amino acids.

In some embodiments, the antibody or fragment thereof comprises a light chain variable region comprising the amino acid sequence of SEQ ID NO: 10 or a sequence at least about 90% identical thereto, e.g., at least about 95, 96, 97, 98, or 99% identical thereto. In some embodiments, the antibody or fragment thereof comprises a light chain variable region comprising at least about 50 contiguous amino acids of the amino acid sequence of SEQ ID NO: 10 or a sequence at least about 90% identical thereto, e.g., at least about 100, 150, or 200 or more contiguous amino acids.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto. Further, the antibody or fragment thereof comprises a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to, e.g., at least about 95, 96, 97, 98, or 99% identical to, SEQ ID NO: 5, SEQ ID NO: 6, SEQ ID NO: 7, SEQ ID NO: 8, SEQ ID NO: 9, or SEQ ID NO: 10.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto. Further, the antibody or fragment thereof comprises a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to, e.g., at least about 95, 96, 97, 98, or 99% identical to, SEQ ID NO: 5, SEQ ID NO: 6, SEQ ID NO: 7, SEQ ID NO: 8, SEQ ID NO: 9, or SEQ ID NO: 10.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto. Further, the antibody or fragment thereof comprises a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to, e.g., at least about 95, 96, 97, 98, or 99% identical to, SEQ ID NO: 5, SEQ ID NO: 6, SEQ ID NO: 7, SEQ ID NO: 8, SEQ ID NO: 9, or SEQ ID NO: 10.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto. Further, the antibody or fragment thereof comprises a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to, e.g., at least about 95, 96, 97, 98, or 99% identical to, SEQ ID NO: 5, SEQ ID NO: 6, SEQ ID NO: 7, SEQ ID NO: 8, SEQ ID NO: 9, or SEQ ID NO: 10.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 5 or a sequence at least about 90% identical thereto, e.g., at least about 95, 96, 97, 98, or 99% identical thereto.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 6 or a sequence at least about 90% identical thereto, e.g., at least about 95, 96, 97, 98, or 99% identical thereto.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 7 or a sequence at least about 90% identical thereto, e.g., at least about 95, 96, 97, 98, or 99% identical thereto.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 8 or a sequence at least about 90% identical thereto, e.g., at least about 95, 96, 97, 98, or 99% identical thereto.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 9 or a sequence at least about 90% identical thereto, e.g., at least about 95, 96, 97, 98, or 99% identical thereto.

In some embodiments, the antibody or fragment thereof comprises a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to, e.g., at least about 95, 96, 97, 98, or 99% identical to, SEQ ID NO: 1, SEQ ID NO: 2, SEQ ID NO: 3, or SEQ ID NO: 4. Further, the antibody or fragment thereof comprises a light chain variable region comprising the amino acid sequence of SEQ ID NO: 10 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 5 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 5 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 5 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 5 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 6 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 7 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 8 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 9 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 1 or a sequence at least about 90% identical thereto, e.g., at least about 95, 96, 97, 98, or 99% identical thereto, and a light chain variable region comprising the amino acid sequence of SEQ ID NO: 10 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 6 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 7 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 8 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 9 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 2 or a sequence at least about 90% identical thereto, e.g., at least about 95, 96, 97, 98, or 99% identical thereto, and a light chain variable region comprising the amino acid sequence of SEQ ID NO: 10 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 6 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 7 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 8 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 9 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 3 or a sequence at least about 90% identical thereto, e.g., at least about 95, 96, 97, 98, or 99% identical thereto, and a light chain variable region comprising the amino acid sequence of SEQ ID NO: 10 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 6 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 7 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 8 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 9 or a sequence at least about 90% identical thereto, e.g., at least about 95, 96, 97, 98, or 99% identical thereto.

In some embodiments, the antibody or fragment thereof comprises a heavy chain variable region comprising the amino acid sequence of SEQ ID NO: 4 or a sequence at least about 90% identical thereto, e.g., at least about 95, 96, 97, 98, or 99% identical thereto, and a light chain variable region comprising the amino acid sequence of SEQ ID NO: 10 or a sequence at least about 90% identical thereto, e.g., at least about 95, 96, 97, 98, or 99% identical thereto.

In some embodiments, the antibodies or fragments thereof described above may be included in compositions. In some embodiments, the compositions are pharmaceutical compositions comprising the antibodies or fragments thereof in a pharmaceutically acceptable carrier, optionally containing one or more medicinal agents, pharmaceutical agents, carriers, adjuvants, dispersing agents, diluents, and the like. As used herein, the term “pharmaceutically acceptable,” with respect to formulation components such as carriers, excipients, and diluents, refers to those components which are not deleterious to a patient and which are compatible with other ingredients, active ingredients, salts, or components. “Pharmaceutically acceptable” includes “veterinarily acceptable,” and thus includes both human and non-human mammal applications independently.

More specifically, as used herein, the term “pharmaceutical composition” refers to a pharmaceutical preparation that contains an antibody or fragment thereof of the present disclosure and that is suitable for administration to a patient for therapeutic purposes. As used herein, the term “patient” refers to a living organism that is treated with an antibody or fragment thereof of the present disclosure, including without limitation any mammal such as, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

In some embodiments, the pharmaceutical composition may include at least one pharmaceutically acceptable component to provide an improved formulation of a compound of the present disclosure, including without limitation one or more pharmaceutically acceptable carriers, excipients or diluents. The carrier, excipient, or diluent may take a wide variety of forms depending on the form of preparation desired for administration.

As used herein, the term “carrier” includes without limitation calcium carbonate, calcium phosphate, various sugars, such as lactose, glucose, or sucrose, types of starch, cellulose derivatives, gelatin, lipids, liposomes, nanoparticles, physiologically acceptable liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like.

As used herein, the term “excipient” generally includes without limitation fillers, binders, disintegrants, glidants, lubricants, complexing agents, solubilizers, stabilizer, preservatives, and surfactants, which may be chosen to facilitate administration of the compound by a particular route. Suitable excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil, mineral oil, polyethylene glycol (e.g., PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g., 2-hydroxypropyl-delta-cyclodextrin), polysorbates (e.g., polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g., a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

As the skilled artisan understands, any diluent known in the art may be utilized in accordance with the present disclosure. In some embodiments of the present disclosure, the diluent is water soluble. In some embodiments of the present disclosure, the diluent is water insoluble. As used herein, the term “diluent” includes without limitation water, saline, phosphate buffered saline (PBS), dextrose, glycerol, ethanol, buffered sodium or ammonium acetate solution, or the like, and combinations thereof.

In some embodiments, the pharmaceutical compositions of the present disclosure include at least one additional active ingredient. As used herein, the term “active ingredient” refers to a therapeutically active compound, as well as any prodrugs thereof and pharmaceutically acceptable salts, hydrates, and solvates of the compound and the prodrugs. Additional active ingredients may be combined with an antibody or fragment thereof of the present disclosure and may be either administered separately or in the same pharmaceutical composition. The amount of additional active ingredients to be given may be determined by one skilled in the art based upon therapy with an antibody or fragment thereof of the present disclosure.

In some embodiments, the pharmaceutical composition is a human pharmaceutical composition. As used herein, the term “human pharmaceutical composition” refers to a pharmaceutical composition intended for administration to a human.

The pharmaceutical compositions of the present disclosure are suitable for administration to a patient by any suitable means, including without limitation those means used to administer conventional medicaments. The pharmaceutical compositions of the present disclosure may be administered using any applicable route that would be considered by one of ordinary skill, including without limitation oral, intravenous (“IV”) injection or infusion, intravesical, subcutaneous (“SC”), intramuscular (“IM”), intraperitoneal, intradermal, intraocular, inhalation (and intrapulmonary), intranasal, transdermal, epicutaneously, subdermal, topical, mucosal, nasal, ophthalmic, impression into skin, intravaginal, intrauterine, intracervical, and rectal. Such dosage forms should allow an antibody or fragment thereof of the present disclosure to reach target cells. Other factors are well known in the art and include considerations such as toxicity and dosage forms that retard a compound or composition from exerting its effects. Techniques and formulations generally may be found in Remington: The Science and Practice of Pharmacy, 21st edition, Lippincott, Williams and Wilkins, Philadelphia, Pa., 2005.

In some embodiments, the pharmaceutical compositions of the present disclosure are adapted for topical administration. As used herein, the term “topical administration” refers to administration of an antibody or fragment thereof of the present disclosure to the skin surface of a patient so that an antibody or fragment thereof of the present disclosure passes through the skin layer. Transdermal administration and transmucosal administration are also encompassed within the term topical administration. As used herein, the term “transdermal” refers to passage of an antibody or fragment thereof of the present disclosure across at least one skin layer of a patient. As used herein, “transmucosal” refers to passage of an antibody or fragment thereof of the present disclosure across a mucous membrane of a patient. Unless otherwise stated or implied, the terms “topical administration,” “transdermal administration,” and “transmucosal administration” are used interchangeably herein.

A variety of topical delivery systems for delivering bioactive compounds to microbes in a patient are well known in the art. Such systems include without limitation lotions, creams, gels, oils, ointments, solutions, suspensions, emulsions, and the like by choice of appropriate carriers in the art. In some embodiments, the pharmaceutical composition is administered in the form of a gel including a polyhydric alcohol.

Suitable carriers include without limitation vegetable or mineral oils, white petrolatum (e.g., white soft paraffin), branched chain fats or oils, animal fats and high molecular weight alcohol (e.g., greater than Cu). In some embodiments, carriers are selected such that an antibody or fragment thereof of the present disclosure is soluble. In some embodiments, emulsifiers, stabilizers, humectants, and antioxidants may also be included as well as agents imparting color or fragrance, if desired. In some embodiments, an organic solvent or co-solvent such as ethanol or propanol may be employed in the pharmaceutical compositions of the present disclosure. In some embodiments, evaporation of the solvent leaves a residue on the treated surface. In some embodiments, penetrants appropriate to the barrier to be permeated are used. Such penetrants are generally known in the art and include without limitation bile salts and fusidic acid derivatives. In some embodiments, detergents may be used to facilitate permeation. In some embodiments, creams for topical administration are formulated from a mixture of mineral oil, self-emulsifying beeswax, and water in which mixture an antibody or fragment thereof of the present disclosure, dissolved in a small amount of solvent (e.g., an oil), is admixed. The specific topical delivery system used depends on the intended site of administration.

In some embodiments, other materials may also be added to the topical pharmaceutical compositions of the present disclosure to have moisturizing effects and to improve the consistency of the pharmaceutical composition. Examples of such compounds include without limitation cetyl esters wax, stearyl alcohol, cetyl alcohol, glycerin, methyl paraben, propyl paraben, quaternium-15, humectants, volatile methylsiloxane fluids, and polydiorganosiloxane-polyoxyalkylene. See, e.g., U.S. Pat. Nos. 5,153,230 and 4,421,769. If it is desirable for the pharmaceutical composition to have additional cleaning effects in some embodiments, chemicals such as sodium lauryl sulfate or a metal salt of a carboxylic acid may be added.

In some embodiments, a wide variety of nonvolatile emollients are useful in the pharmaceutical compositions of the present disclosure. Non-limiting examples of such nonvolatile emollients are listed in McCutcheon's, Vol. 2 Functional Materials, North American Edition, (1992), pp. 137-168, and CTFA Cosmetic Ingredient Handbook, Second Edition (1992) which lists Skin-Conditioning Agents at pp. 572-575 and Skin Protectants at p. 580. In some embodiments, the nonvolatile emollients include silicones, hydrocarbons, esters, and mixtures thereof. In some embodiments, the esters include esters of monofunctional and difunctional fatty acids that have been esterified with alcohols and polyols (i.e., alcohols having two or more hydroxyl groups). In some embodiments, long chain esters of long chain fatty acids are utilized in the pharmaceutical compositions of the present disclosure (i.e., C10-40 fatty acids esterified with C10-40 fatty alcohols). Non-limiting examples of esters useful in the pharmaceutical compositions of the present disclosure include without limitation those selected from the group consisting of diisopropyl adipate, isopropyl myristate, isopropyl palmitate, myristyl propionate, ethylene glycol distearate, 2-ethylhexyl palmitate, isodecyl neopentanoate, C12-15 alcohol benzoate, di-2-ethylhexyl maleate, ceryl palmitate, myristyl myristate, stearyl stearate, cetyl stearate, behenyl behenrate, and mixtures thereof.

Examples of silicone emollients useful in the pharmaceutical compositions of the present disclosure include without limitation polyalkylsiloxanes, cyclic polyalkylsiloxanes, and polyalkylarylsiloxanes. Suitable commercially available polyalkylsiloxanes include the polydimethylsiloxanes, which are also known as dimethicones, non-limiting examples of which include the VICASIL™ series sold by General Electric Company and the DOW CORNING™ 200 series sold by Dow Corning Corporation. Commercially available polyalkylsiloxanes include cyclomethicones (DOW CORNING™ 244 fluid), DOW CORNING™ 344 fluid, DOW CORNING™ 245 fluid and DOW CORNING™ 345), among others. A suitable commercially available trimethylsiloxysilicate is sold as a mixture with dimethicone as DOW CORNING™ 593 fluid. Also useful in the pharmaceutical compositions of the present disclosure are dimethiconols, which are hydroxyl terminated dimethyl silicones. Suitable commercially available dimethiconols are typically sold as mixtures with dimethicone or cyclomethicone (e.g., DOW CORNING™ 1401, 1402, and 1403 fluids). Suitable commercially available polyalkylarylsiloxanes include SF1075 methylphenyl fluid (sold by General Electric Company) and 556 Cosmetic Grade phenyl trimethicone fluid (sold by Dow Corning Corporation).

Hydrocarbons suitable for use in the pharmaceutical compositions of the present disclosure include without limitation straight and branched chain hydrocarbons having from about 10 to about 30 carbon atoms. In some embodiments, the straight and branched chain hydrocarbons have from about 12 to about 24 carbon atoms. In some embodiments, the straight and branched chain hydrocarbons have from about 16 to about 22 carbon atoms. Non-limiting examples of such hydrocarbon materials include dodecane, squalane, cholesterol, 5 hydrogenated polyisobutylene, docosane (i.e., a C22 hydrocarbon), hexadecane, and isohexadecane (a commercially available hydrocarbon sold as PERMETHYL™ 101A by Presperse, South Plainsfield, N.J.), among others.

In some embodiments, the topical pharmaceutical compositions of the present disclosure include propylene glycol. In some embodiments, propylene glycol acts as a surfactant and assists in penetration, contact, and absorption of an antibody or fragment thereof of the present disclosure. In some embodiments, propylene glycol serves as a preservative. In some embodiments, the pharmaceutical compositions of the present disclosure include a non-ionic surfactant, such as, for example, polysorbate. Such a surfactant provides better surface contact of the pharmaceutical compositions of the present disclosure with mucosa (such as vaginal mucosa) by further reducing surface tension.

The topical pharmaceutical compositions of the present disclosure optionally may also be formulated with a lipophilic phase, such as, for example, emulsions and liposome dispersions. In some embodiments, liposomal formulations may extend circulation time of an antibody or fragment thereof of the present disclosure, increase permeability of an antibody or fragment thereof of the present disclosure, and improve overall efficacy of an antibody or fragment thereof of the present disclosure as medicament. In some embodiments, an antibody or fragment thereof of the present disclosure may be combined with a lipid, cationic lipid or anionic lipid. In some embodiments, the resulting emulsion or liposomal suspension can effectively increase the in vivo half-life of the activity of a pharmaceutical composition of the present disclosure. Examples of suitable anionic lipids for use with the pharmaceutical compositions of the present disclosure include, but are not limited to, cardiolipin, dimyristoyl, dipalmitoyl, dioleoyl phosphatidyl choline, phosphatidyl glycerol, palmitoyloleoyl phosphatidyl choline, phosphatidyl glycerol, phosphatidic acid, lysophosphatidic acid, phosphatidyl serine, phosphatidyl inositol, and anionic forms of cholesterol.

In some embodiments, an antibody or fragment thereof of the present disclosure is incorporated into liposomes. In some embodiments, neutral lipids, cholesterol, and/or polyethylene glycol (PEG) are utilized in such liposomes. In some embodiments, the liposomal composition is composed of partially hydrogenated soy phosphatidylcholine (PHSC), cholesterol, methoxy-terminated PEG (mPEG), and/or distearoyl phosphatidyl ethanolamine (DSPE). The liposomes can be prepared according to any suitable method known in the art.

In some embodiments, topical administration is through nasal sprays or suppositories (rectal or vaginal). Suppositories are prepared by mixing an antibody or fragment thereof of the present disclosure with a lipid vehicle such as Theobroma oil, cacao butter, glycerin, gelatin, polyoxyethylene glycols, and the like. In some embodiments, topical administration comprises a transdermal patch or dressing such as a bandage impregnated with an antibody or fragment thereof of the present disclosure and optionally one or more carriers, excipients or diluents known in the art. In some embodiments, such dressings include without limitation semipermeable films, foams, hydrocolloids, and calcium alginate swabs. In some embodiments, the dosage administration will be continuous rather than intermittent throughout the dosage regimen.

In some embodiments, the pharmaceutical compositions of the present disclosure are adapted for oral administration. As used herein, the term “oral administration” refers to administration of an antibody or fragment thereof of the present disclosure to the mouth of a patient for ingestion into the gastrointestinal tract. In some embodiments, the pharmaceutical compositions of the present disclosure can be formulated into conventional oral dosage forms including without limitation capsules, tablets, powders, and liquid preparations such as suspensions, solutions, elixirs, syrups, concentrated drops, and the like. In some embodiments, an antibody or fragment thereof of the present disclosure may be combined with solid excipients, optionally grinding a resulting mixture, and optionally processing the mixture of granules, after adding suitable auxiliaries, if desired, to obtain, for example, tablets, coated tablets, hard capsules, soft capsules, solutions (e.g., aqueous, alcoholic or oily solutions), and the like. In some embodiments, excipients suitable for use in the oral pharmaceutical compositions of the present disclosure include without limitation fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP or povidone); and oily excipients, including vegetable and animal oils, such as sunflower oil, olive oil, or cod liver oil. In some embodiments, the oral pharmaceutical compositions of the present disclosure may also contain disintegrating agents, such as, for example, cross-linked polyvinylpyrrolidone, agar, or alginic acid, or a salt thereof such as sodium alginate; a lubricant, such as talc or magnesium stearate; a plasticizer, such as glycerol or sorbitol; a sweetening agent such as sucrose, fructose, lactose, or aspartame; a natural or artificial flavoring agent, such as, for example, peppermint, oil of wintergreen, or cherry flavoring; or dye-stuffs or pigments, which may be used for identification or characterization of different doses or combinations. In some embodiments, the oral pharmaceutical compositions of the present disclosure may also contain dragée cores with suitable coatings. In some embodiments, concentrated sugar solutions may be used, which may optionally contain, for example, gum arabic, talc, poly-vinylpyrrolidone, carbopol gel, polyethylene glycol, titanium dioxide, lacquer solutions, and suitable organic solvents or solvent mixtures.

In some embodiments, the pharmaceutical compositions of the present disclosure that can be used orally include without limitation push-fit capsules made of gelatin (“gelcaps”), as well as soft, sealed capsules made of gelatin, and a plasticizer, such as glycerol or sorbitol. In some embodiments, the push-fit capsules can contain an antibody or fragment thereof of the present disclosure in admixture with filler such as lactose, binders such as starches, and/or lubricants such as talc or magnesium stearate and, optionally, stabilizers. In some embodiments including soft capsules, the an antibody or fragment thereof may be dissolved or suspended in suitable liquids, such as fatty oils, liquid paraffin, liquid polyethylene glycols, and the like.

In some embodiments, the pharmaceutical compositions of the present disclosure are adapted for inhalation administration. As used herein, the term “inhalation administration” refers to delivery of an antibody or fragment thereof of the present disclosure by passage through a patient's nose or mouth during inhalation and passage of the antibody or fragment thereof through the walls of the lungs of the patient. In some embodiments, the pharmaceutical compositions of the present disclosure suitable for inhalation administration may be formulated as dry powder or a suitable solution, suspension, or aerosol. In some embodiments, powders and solutions may be formulated with suitable additives known in the art. In some embodiments, powders may include a suitable powder base such as lactose or starch. In some embodiments, solutions may comprise propylene glycol, sterile water, ethanol, sodium chloride, and other additives, such as, for example, acid, alkali, and buffer salts. In some embodiments, such solutions or suspensions may be administered by inhaling via a spray, pump, atomizer, nebulizer, and the like. In some embodiments, the pharmaceutical compositions of the present disclosure suitable for inhalation administration may also be used in combination with other inhaled therapies, including without limitation corticosteroids such as, for example, fluticasone proprionate, beclomethasone dipropionate, triamcinolone acetonide, budesonide, and mometasone furoate; beta agonists such as, for example, albuterol, salmeterol, and formoterol; anticholinergic agents such as, for example, ipratroprium bromide or tiotropium; vasodilators such as, for example, treprostinal and iloprost; enzymes such as, for example, DNAase; therapeutic proteins; immunoglobulin antibodies; an oligonucleotide, such as, for example, single or double stranded DNA or RNA, siRNA; antibiotics such as, for example, tobramycin; muscarinic receptor antagonists; leukotriene antagonists; cytokine antagonists; protease inhibitors; cromolyn sodium; nedocril sodium; and sodium cromoglycate.

In some embodiments, the pharmaceutical compositions of the present disclosure are adapted for intravesical administration. As used herein, the term “intravesical administration” refers to delivery of an antibody or fragment thereof of the present disclosure directly into the bladder of a patient. In some embodiments, the pharmaceutical composition is administered via a catheter. In some embodiments, the catheter is a urethral catheter.

In some embodiments, the pharmaceutical compositions of the present disclosure are adapted for parenteral administration. As used herein, the term “parenteral administration” refers to an antibody or fragment thereof of the present disclosure being injected or infused into a patient and includes without limitation intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the pharmaceutical compositions of the present disclosure suitable for parenteral administration may be formulated in sterile liquid solutions, including without limitation physiologically compatible buffers or solutions, such as, for example, saline solution, Hank's solution or Ringer's solution. In some embodiments, the pharmaceutical compositions of the present disclosure suitable for parenteral administration may be prepared as dispersions in non-aqueous solutions, such as, for example, glycerol, propylene glycol, ethanol, liquid polyethylene glycols, triacetin, vegetable oils, and the like. In some embodiments, solutions may also contain a preservative, such as, for example, methylparaben, propylparaben, chlorobutanol, phenol, sorbic acid, thimerosal, and the like. In addition, pharmaceutical compositions of the present disclosure suitable for parenteral administration may be formulated in solid form, including, for example, lyophilized forms, and redissolved or suspended prior to use. In some embodiments, the pharmaceutical composition is administered via a needle. For injection, the carrier may be a liquid, such as sterile pyrogen-free water, pyrogen-free phosphate-buffered saline solution, bacteriostatic water, or CREMOPHOR EL® (BASF, Parsippany, N.J.). For other methods of administration, the carrier can be either solid or liquid.

The present disclosure also provides a kit. In some embodiments, the kit comprises an antibody or fragment thereof according to the present disclosure. As used herein, the term “kit” refers to any manufacture, such as, for example, a package, container, and the like, containing an antibody or fragment thereof according to the present disclosure. In some embodiments, an antibody or fragment thereof according to the present disclosure is packaged in a vial, bottle, tube, flask, or patch, which may be further packaged within a box, envelope, bag, or the like. In some embodiments, an antibody or fragment thereof according to the present disclosure is approved by the U.S. Food and Drug Administration or similar regulatory agency in the U.S. or a jurisdiction or territory outside the U.S. for administration to a patient. In some embodiments, the kit includes written instructions for use and/or other indication that an antibody or fragment thereof according to the present disclosure is suitable or approved for administration to a patient. In some embodiments, an antibody or fragment thereof of the present disclosure is packaged in unit dose or single unit dose form, such as, for example, single unit dose pills, capsules or the like. In some embodiments, the kit includes a dispenser.

The present disclosure also provides the use of an antibody or fragment thereof of the present disclosure for the manufacture of a medicament. As used herein, the term “medicament” refers to a pharmaceutical composition according to the present disclosure. In some embodiments, the pharmaceutical composition is contained in any manufacture, such as, for example, a package, container, and the like.

As one aspect, methods of depleting, inhibiting, or depleting and inhibiting pDC in a patient include delivering to the patient an effective amount of the antibody or fragment thereof that specifically binds to an epitope comprising BDCA2 and depletes, inhibits, or depletes and inhibits pDC, thereby depleting, inhibiting, or depleting and inhibiting pDC. The antibody or fragment thereof is as described above. Additionally, the antibody or fragment thereof may be formulated in a pharmaceutical composition as described above.

The term “deplete,” as used herein with respect to pDC, refers to a measurable decrease in the number of pDC in a patient or in a sample. The reduction can be at least about 10%, e.g., at least about 20%, 30%, 40%, 50%, 60%, 70%, 80%, 90%, 95%, 96%, 97%, 98%, 99%, or more. In certain embodiments, the term refers to a decrease in the number of pDC in a patient or in a sample to an amount below detectable limits. In some embodiments, pDC are depleted by at least about 50% relative to patients that have not received the antibody or fragment thereof, e.g., at least about 60%, 70%, 80%, 90%, 95%, 96%, 97%, 98%, or 99% or more. In other embodiments, the present disclosure provides methods of reducing the number of and/or depleting pDC in a sample ex vivo or in vitro, e.g., a mixed population of cells, comprising delivering to the sample an effective amount of an antibody or a fragment thereof that specifically binds to BDCA2 and reduces the number of and/or depletes pDC, thereby reducing the number of and/or depleting pDC. In some embodiments, the BDCA2 is human BDCA2.

The term “inhibit,” as used herein with respect to pDC, refers to slowing or eliminating some activity of the pDC, such as cellular survival, pDC activation (for example, Type 1 interferon production), and any other activity of the cells. Various methods of quantifying and/or qualifying cell inhibition are within the purview of one of ordinary skill.

In some embodiments, a method of treating a disorder associated with pDC in a patient in need thereof includes delivering to the patient an effective amount of an antibody or a fragment thereof that specifically binds to an epitope comprising blood dendritic cell antigen-2 (BDCA2) and depletes, inhibits, or depletes and inhibits pDC, thereby treating the disorder. The antibody or fragment thereof is as described above. Additionally, the antibody or fragment thereof may be formulated in a pharmaceutical composition as described above.

The present disclosure provides methods of treatment. As used herein, the terms “treating,” “treatment,” “therapy,” and like terms refer to administration of an antibody or fragment thereof or a pharmaceutical composition of the present disclosure in an amount effective to prevent, alleviate or ameliorate one or more symptoms of a disease or condition (i.e., indication) and/or to prolong the survival of the patient being treated. In some embodiments, “treating,” “treatment,” “therapy,” and like terms also include without limitation reducing or eliminating infection in a patient.

In carrying out the methods of the present disclosure, an effective amount of an antibody or fragment thereof of the present disclosure is administered to a patient in need thereof. As used herein, the term “effective amount,” in the context of administration, refers to the amount of an antibody or fragment thereof or pharmaceutical composition of the present disclosure that when administered to a patient is sufficient to prevent, alleviate or ameliorate one or more symptoms of a disease or condition (i.e., indication) and/or to prolong the survival of the patient being treated. Such an amount should result in no or few adverse events in the treated patient. Similarly, such an amount should result in no or few toxic effects in the treated patient. As those familiar with the art will understand, the amount of an antibody or fragment thereof or pharmaceutical composition of the present disclosure will vary depending upon a number of factors, including without limitation the activity of an antibody or fragment thereof of the present disclosure (in vitro, e.g. a compound of the present disclosure vs. target, or in vivo activity in animal efficacy models), pharmacokinetic results in animal models (e.g., biological half-life or bioavailability), the type of patient being treated, the patient's age, size, weight, and general physical condition, the disorder associated with the patient, and the dosing regimen being employed in the treatment. Those skilled in the art will appreciate that the therapeutic effects need not be complete or curative, as long as some benefit is provided to the patient.

In some embodiments of the present disclosure, an effective amount of an antibody or fragment thereof of the present disclosure to be delivered to a patient in need thereof can be quantified by determining micrograms of an antibody or fragment thereof of the present disclosure per kilogram of patient body weight. In some embodiments, the amount of an antibody or fragment thereof of the present disclosure administered to a patient is from about 0.1 to about 1000 milligram (mg) of an antibody or fragment thereof of the present disclosure per kilogram (kg) of patient body weight. In some embodiments, the amount of an antibody or fragment thereof of the present disclosure administered to a patient is from about 0.1 to about 500 mg of an antibody or fragment thereof of the present disclosure per kg of patient body weight. In some embodiments, the amount of an antibody or fragment thereof of the present disclosure administered to a patient is from about 0.1 to about 300 mg of an antibody or fragment thereof of the present disclosure per kg of patient body weight. In some embodiments, the amount of an antibody or fragment thereof of the present disclosure administered to a patient is from about 0.1 to about 200 mg of an antibody or fragment thereof of the present disclosure per kg of patient body weight. In some embodiments, the amount of an antibody or fragment thereof of the present disclosure administered to a patient is from about 0.1 to about 100 mg of an antibody or fragment thereof of the present disclosure per kg of patient body weight. In some embodiments, the amount of an antibody or fragment thereof of the present disclosure administered to a patient is from about 0.1 to about 80 mg of an antibody or fragment thereof of the present disclosure per kg of patient body weight. In some embodiments, the amount of an antibody or fragment thereof of the present disclosure administered to a patient is from about 0.1 to about 50 mg of an antibody or fragment thereof of the present disclosure per kg of patient body weight. In some embodiments, the amount of an antibody or fragment thereof of the present disclosure administered to a patient is from about 0.1 to about 20 mg of an antibody or fragment thereof of the present disclosure per kg of patient body weight. In some embodiments, the amount of an antibody or fragment thereof of the present disclosure administered to a patient is from about 0.1 to about 10 mg of an antibody or fragment thereof of the present disclosure per kg of patient body weight. As those of ordinary skill in the art understand multiple doses may be used.

In some embodiments of the present disclosure, an antibody or fragment thereof of the present disclosure is administered as a multiple dose regimen. As used herein, the term “multiple dose regimen” refers to a treatment time period of more than one day. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. In some embodiments of the present disclosure, the multiple dose regimen is a time period of up to about five months. In some embodiments of the present disclosure, the multiple dose regimen is a time period of up to about six months. Other time periods may be used herein.

In some embodiments of the present disclosure, an antibody or fragment thereof of the present disclosure is administered as part of a chronic treatment regimen. As used herein, the term “chronic treatment regimen” refers to treatment with an antibody or fragment thereof of the present disclosure over an extended period of time during a patient's lifetime. In some embodiments, chronic treatment is lifelong treatment.

In some embodiments of the present disclosure, an antibody or fragment thereof of the present disclosure is administered as a single dose. In some embodiments of the present disclosure, an antibody or fragment thereof of the present disclosure is administered as a single unit dose. As used herein, the term “unit dose” is a predetermined amount of an antibody or fragment thereof of the present disclosure. The amount of an antibody or fragment thereof of the present disclosure is generally equal to the dosage of an antibody or fragment thereof of the present disclosure that would be administered to a patient or a convenient fraction of such a dosage such as, for example, one-half or one-third of such a dosage. According to the methods of the present disclosure, the terms “single dose” and “single unit dose” include embodiments wherein an antibody or fragment thereof of the present disclosure can be administered as a single application and administered as multiple applications.

In some embodiments, an antibody or fragment thereof of the present disclosure may also be used in combination with one or more additional active ingredients for treating the same disease or condition. In some embodiments, such combination use includes administration of an antibody or fragment thereof of the present disclosure and one or more additional active ingredient at different times, or co-administration of an antibody or fragment thereof of the present disclosure and one or more additional active ingredients. In some embodiments, dosage may be modified for an antibody or fragment thereof of the present disclosure or one or more additional active ingredients used in combination, e.g., reduction in the amount dosed relative to an antibody or fragment thereof of the present disclosure or one or more additional active ingredients used alone, by methods well known to those of ordinary skill in the art. In some embodiments, co-administration includes simultaneous administration of an antibody or fragment thereof of the present disclosure and an additional active ingredient in the same dosage form, simultaneous administration of an antibody or fragment thereof of the present disclosure and an additional active ingredient in separate dosage forms, and separate administration of an antibody or fragment thereof of the present disclosure and an additional active ingredient.

It is understood that use in combination includes use with one or more additional active ingredients or other medical procedure in which the one or more additional active ingredients or other medical procedure may be administered at different times (e.g., within a short time, such as within hours (e.g., about 1, 2, 3, 4-24 hours, etc.), or within a longer time (e.g. about 1-2 days, 2-4 days, 4-7 days, 1-4 weeks, etc.)) than an antibody or fragment thereof or pharmaceutical composition of the present disclosure, or at the same time as an antibody or fragment thereof or pharmaceutical composition of the present disclosure. Use in combination also includes use with one or more additional active ingredients or other medical procedure that is administered once or infrequently, such as surgery, along with an antibody or fragment thereof or pharmaceutical composition of the present disclosure administered within a short time or longer time before or after the administration of the one or more additional active ingredients or completion of the other medical procedure.

In some embodiments, the present disclosure provides for delivery of an antibody or fragment thereof or pharmaceutical composition of the present disclosure and one or more additional active ingredients delivered by a different route of administration or by the same route of administration. In some embodiments, the use in combination for any route of administration includes delivery of an antibody or fragment thereof or pharmaceutical composition of the present disclosure and one or more additional active ingredients delivered by the same route of administration together in any pharmaceutical composition, including pharmaceutical compositions in which the two compounds are chemically linked in such a way that such compounds maintain their therapeutic activity when administered. In some embodiments, the one or more additional active ingredients may be co-administered with an antibody or fragment thereof or pharmaceutical composition of the present disclosure. In some embodiments, use in combination by co-administration includes administration of co-formulations or formulations of chemically joined compounds, or administration of two or more compounds in separate formulations within a short time of each other (e.g., within about an hour, 2 hours, 3 hours, up to about 24 hours, etc.), administered by the same or different routes. In some embodiments, co-administration of separate formulations includes co-administration by delivery via one device, for example, the same inhalant device, the same syringe, etc., or administration from separate devices within a short time of each other. In some embodiments, co-formulations of an antibody or fragment thereof or pharmaceutical composition of the present disclosure and one or more additional active ingredients delivered by the same route includes preparation of the materials together such that they can be administered by one device, including the separate compounds combined in one formulation, or compounds that are modified such that the compounds are chemically joined, yet still maintain their biological activity. In some embodiments, such chemically joined compounds may have a linkage that is substantially maintained in vivo, or the linkage may break down in vivo, separating the two active components.

The phrase “disorder associated with pDC,” as used herein, refers to any disease, disorder, or condition in which pDC play a role in a cause, side effect, symptom, or other aspect in the disease, disorder, or condition. Examples of such disorders include, without limitation, an autoimmune disease, an inflammatory condition, a respiratory condition, a skin disorder, a cancer, and a combination of two or more of these.

The term “infectious diseases,” as used herein, refers to any disease associated with infection by an infectious agent. Examples of infectious agents include, without limitation, viruses and microorganisms. Viruses include, without limitation, Hepadnaviridae including hepatitis A, B, C, D, E, F, G, etc.; Flaviviridae including human hepatitis C virus (HCV), yellow fever virus and dengue viruses; Retroviridae including human immunodeficiency viruses (HIV) and human T lymphotropic viruses (HITLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses; Poxviridae including Orf virus, molluscum contageosum virus, smallpox virus and Monkey pox virus; Togaviridae including Venezuelan equine encephalitis virus; Coronaviridae including corona viruses such as a severe acute respiratory syndrome (SARS) virus (e.g., SARS-CoV-2); and Picornaviridae including polioviruses; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, Canine distemper virus, Canine contagious hepatitis virus, Feline calicivirus, Feline rhinotracheitis virus, TGE virus (swine), Foot and mouth disease virus, simian virus 5, human parainfluenza virus type 2, human metapneuomovirus, enteroviruses, and any other pathogenic virus now known or later identified (see, e.g., Fundamental Virology, Fields et al., Eds., 3d ed., Lippincott-Raven, New York, 1996, the entire content of which is incorporated by reference herein for the teachings of pathogenic viruses).

Pathogenic microorganisms include, but are not limited to, Rickettsia, Chlamydia, Mycobacteria, Clostridia, Corynebacteria, Mycoplasma, Ureaplasma, Legionella, Shi-gella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4th ed., Lippincott, New York, 1990, the entire content of which is incorporated herein by reference for the teachings of pathogenic microorganisms). Specific examples of microorganisms include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum. Bacillus anthracis, Salmonella typhi, Vibrio cholera. Pasteurella pestis (Yersinia pestis), Pseudomonas aeruginosa, Campylobacter jejuni, Clostridium dificile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

The term “autoimmune disorders,” as used herein, refers to any disorder associated with an autoimmune reaction. Examples include, without limitation, diabetes, cutaneous psoriasis, hyperthyroidism, autoimmune adrenal insufficiency, hemolytic anemia, rheumatic carditis, systemic lupus erythematosus, cutaneous lupus erythematosis, psoriatic arthritis, Sjogren's syndrome polymyositis, myositis, multiple sclerosis, Crohn's disease, ulcerative colitis, lupus, inflammatory bowel syndrome, dermatomyositis, dermatitis, atopic dermatitis, scleroderma, polymyositis, psoriasis, alopecia areata, rheumatoid arthritis, graft-versus-host disease, interferonopathies, and irritable bowel syndrome.

The term “inflammatory condition,” as used herein, refers to a condition in which tissue of an organism undergoes a response to certain stimuli, including an autoimmune disorder, which is generally referred to as “inflammation.” Symptoms of inflammation include swelling, pain, heat, redness, and loss of function of the tissue. Exemplary inflammatory conditions include neurodegenerative diseases; diabetes-associated nephropathy and retinopathy; protein wasting, muscle fatigue or inflammation; infectious diseases; various cardiovascular diseases or disorders, including atherosclerosis; neurodegenerative diseases such as Alzheimer's disease; infectious disease, such as, for example, myocarditis, cardiomyopathy, acute endocarditis, pericarditis; Systemic Inflammatory Response Syndrome (SIRS)/sepsis; adult respiratory distress syndrome (ARDS); asthma; rheumatoid arthritis; osteoarthritis; juvenile arthritis; dry eye syndrome; systemic lupus erythematosis; airway hyperresponsiveness (AHR); bronchial hyperreactivity; chronic obstructive pulmonary disease (COPD); congestive heart failure (CHF); inflammatory complications of diabetes mellitus; metabolic syndrome; ankylosis spondylitis; end stage renal disease (ESRD); hepatitis; liver fibrosis; lung fibrosis organ transplant rejection; acute kidney injury; and aging-related diseases.

The term “respiratory condition,” as used herein, refers to a pathological condition affecting the organs and tissues that make gas exchange difficult in air-breathing animals. They include conditions of the respiratory tract including the trachea, bronchi, bronchioles, alveoli, pleurae, pleural cavity, the nerves, and muscles of respiration. Respiratory diseases range from mild and self-limiting, such as the common cold, influenza, and pharyngitis to life-threatening diseases such as bacterial pneumonia, pulmonary embolism, tuberculosis, acute or chronic asthma, lung fibrosis, lung cancer, and severe acute respiratory syndromes, such as COVID-19. Respiratory diseases may be classified in many different ways, including by the organ or tissue involved, by the type and pattern of associated signs and symptoms, or by the cause of the disease.

The term “skin disorder,” as used herein, refers to any medical condition that affects the integumentary system of a patient—the organ system that encloses the body and includes skin, hair, nails, and related muscle and glands, the major function of which is as a barrier against the external environment. Exemplary skin disorders include, without limitation, scleroderma, psoriasis, dermatomyositits, and atopic dermatitis.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, B-cell acute lymphocytic leukemia, blastic plasmacytoid dendritic cell neoplasm (“BPDCN” or “pDC leukemia”), chronic lymphocytic leukemia, acute myelogenous leukemia, chronic myelogenous leukemia (“AML”), chronic granulocytic leukemia, acute granulocytic leukemia, aging-related hairy cell leukemia, chronic myelomonocytic leukemia (“CMML”), myelodysplastic syndrome, chronic myelofibrosis, multiple myeloma, neuroblastoma, rhabdomyosarcoma, Kaposi's sarcoma, Waldenström's macroglobulinemia, diffuse large B-cell lymphoma (DLBCL) (L265P), polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers.

In addition to the aspects and embodiments described and provided elsewhere in the present disclosure, the following non-limiting list of embodiments are also contemplated.
1. An antibody or fragment thereof comprising:
   a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
2. The antibody or fragment there of according to clause 1, wherein the antibody or fragment thereof binds an epitope having SEQ ID NO: 11 with an affinity (K_{d}) from 0.1 nanomolar (nM) to 100 nM.
3. The antibody or fragment thereof according to clause 1 or clause 2, wherein the antibody or fragment thereof is a monoclonal antibody or fragment thereof.
4. The antibody or fragment thereof according to clause 1 to 3, wherein the antibody or fragment thereof is a humanized antibody or fragment thereof.
5. The antibody or fragment thereof according to clause 1 to 4, wherein:
   the heavy chain variable region comprises an amino acid sequence given by SEQ ID NO: 1 or a sequence at least about 90% identical to SEQ ID NO: 1; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
6. The antibody or fragment thereof according to clause 1 to 4, wherein:
   the heavy chain variable region comprises an amino acid sequence given by SEQ ID NO: 2 or a sequence at least about 90% identical to SEQ ID NO: 2; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
7. The antibody or fragment thereof according to clause 1 to 4, wherein:
   the heavy chain variable region comprises an amino acid sequence given by SEQ ID NO: 3 or a sequence at least about 90% identical to SEQ ID NO: 3; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
8. The antibody or fragment thereof according to clause 1 to 4, wherein:
   the heavy chain variable region comprises an amino acid sequence given by SEQ ID NO: 4 or a sequence at least about 90% identical to SEQ ID NO: 4; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
9. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 5 or a sequence at least about 90% identical to SEQ ID NO: 5.
10. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 6 or a sequence at least about 90% identical to SEQ ID NO: 6.
11. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 7 or a sequence at least about 90% identical to SEQ ID NO: 7.
12. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 8 or a sequence at least about 90% identical to SEQ ID NO: 8.
13. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 9 or a sequence at least about 90% identical to SEQ ID NO: 9.
14. The antibody or fragment thereof according to clause 1 to 4, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 10 or a sequence at least about 90% identical to SEQ ID NO: 10.
15. The antibody or fragment thereof according to clause 1 to 5 or 9, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 5 or a sequence at least about 90% identical to SEQ ID NO: 5.
16. The antibody or fragment thereof according to clause 1 to 4, 6, or 9, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 5 or a sequence at least about 90% identical to SEQ ID NO: 5.
17. The antibody or fragment thereof according to clause 1 to 4, 7, or 9, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 5 or a sequence at least about 90% identical to SEQ ID NO: 5.
18. The antibody or fragment thereof according to clause 1 to 4, 8, or 9, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 5 or a sequence at least about 90% identical to SEQ ID NO: 5.
19. The antibody or fragment thereof according to clause 1 to 5 or 10, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 6 or a sequence at least about 90% identical to SEQ ID NO: 6.
20. The antibody or fragment thereof according to clause 1 to 5 or 11, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 7 or a sequence at least about 90% identical to SEQ ID NO: 7.
21. The antibody or fragment thereof according to clause 1 to 5 or 12, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 8 or a sequence at least about 90% identical to SEQ ID NO: 8.
22. The antibody or fragment thereof according to clause 1 to 5 or 13, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 9 or a sequence at least about 90% identical to SEQ ID NO: 9.
23. The antibody or fragment thereof according to clause 1 to 5 or 14, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 10 or a sequence at least about 90% identical to SEQ ID NO: 10.
24. The antibody or fragment thereof according to clause 1 to 4, 6, or 10, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 6 or a sequence at least about 90% identical to SEQ ID NO: 6.
25. The antibody or fragment thereof according to clause 1 to 4, 7, or 10, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 6 or a sequence at least about 90% identical to SEQ ID NO: 6.
26. The antibody or fragment thereof according to clause 1 to 4, 8, or 10, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 6 or a sequence at least about 90% identical to SEQ ID NO: 6.
27. The antibody or fragment thereof according to clause 1 to 4, 6, or 11, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 7 or a sequence at least about 90% identical to SEQ ID NO: 7.
28. The antibody or fragment thereof according to clause 1 to 4, 7, or 11, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 7 or a sequence at least about 90% identical to SEQ ID NO: 7.
29. The antibody or fragment thereof according to clause 1 to 4, 8, or 11, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 7 or a sequence at least about 90% identical to SEQ ID NO: 7.
30. The antibody or fragment thereof according to clause 1 to 4, 6, or 12, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 8 or a sequence at least about 90% identical to SEQ ID NO: 8.
31. The antibody or fragment thereof according to clause 1 to 4, 7, or 12, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 8 or a sequence at least about 90% identical to SEQ ID NO: 8.
32. The antibody or fragment thereof according to clause 1 to 4, 8, or 12, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 8 or a sequence at least about 90% identical to SEQ ID NO: 8.
33. The antibody or fragment thereof according to clause 1 to 4, 6, or 13, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 9 or a sequence at least about 90% identical to SEQ ID NO: 9.
34. The antibody or fragment thereof according to clause 1 to 4, 7, or 13, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 9 or a sequence at least about 90% identical to SEQ ID NO: 9.
35. The antibody or fragment thereof according to clause 1 to 4, 8, or 13, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 9 or a sequence at least about 90% identical to SEQ ID NO: 9.
36. The antibody or fragment thereof according to clause 1 to 4, 6, or 14, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 10 or a sequence at least about 90% identical to SEQ ID NO: 10.
37. The antibody or fragment thereof according to clause 1 to 4, 7, or 14, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 10 or a sequence at least about 90% identical to SEQ ID NO: 10.
38. The antibody or fragment thereof according to clause 1 to 4, 8, or 14, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 10 or a sequence at least about 90% identical to SEQ ID NO: 10.
39. A method for depleting, inhibiting, or depleting and inhibiting plasmacytoid dendritic cells (pDC) in a patient in need thereof, the method comprising administering to the patient an effective amount of an antibody or fragment thereof that specifically binds to an epitope comprising blood dendritic cell antigen-2 (BDCA2) and depletes, inhibits, or depletes and inhibits pDC, thereby depleting, inhibiting, or depleting and inhibiting pDC; wherein the antibody or fragment thereof comprises:
   a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
40. The method according to clause 39, wherein the epitope comprises an amino acid sequence given by SEQ ID NO: 11, and the antibody or fragment thereof binds the epitope with an affinity (K_{d}) from 0.1 nanomolar (nM) to 100 nM.
41. The method according to clause 39 or clause 40, wherein the antibody or fragment thereof is a monoclonal antibody or fragment thereof.
42. The method according to clause 39 to 41, wherein the antibody or fragment thereof is a humanized antibody or fragment thereof.
43. The method according to clause 39 to 42, wherein the BDCA2 is human BDCA2.
44. A method for treating a disorder associated with plasmacytoid dendritic cells (pDC) in a patient in need thereof, the method comprising:
administering to the patient an effective amount of an antibody or a fragment thereof that specifically binds to an epitope comprising blood dendritic cell antigen-2 (BDCA2) and depletes, inhibits, or depletes and inhibits pDC, thereby treating the disorder; wherein the antibody or fragment thereof comprises:
   a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
45. The method of clause 44, wherein the disorder is selected from the group consisting of an infectious disease, an autoimmune disease, an inflammatory condition, a respiratory condition, a skin disorder, a cancer, and a combination of two or more of these.
46. The method of clause 45, wherein the disorder is an infectious disease associated with infection by an infectious agent.
47. The method of clause 46, wherein the infectious agent is one or more viruses or one or more microorganisms.
48. The method of clause 47, wherein the infectious agent is a virus selected from the group consisting of Hepadnaviridae; Flaviviridae; Retroviridae; Herpesviridae; Papovaviridae; Rhabdoviridae; Paramyxoviridae; Reoviridae; Bunyaviridae; Filoviridae; Adenoviridae; Parvoviridae; Arenaviridae; Orthomyxoviridae; Poxviridae; Togaviridae; Coronaviridae; Picornaviridae; rhinoviruses; orbiviruses; picodnaviruses; encephalomyocarditis virus (EMV); Parainfluenza viruses; adenoviruses; Coxsackieviruses; Echoviruses; Rubeola virus; Rubella virus; human papillomaviruses; Canine distemper virus; Canine contagious hepatitis virus; Feline calicivirus; Feline rhinotracheitis virus; TGE virus (swine); Foot and mouth disease virus; simian virus 5; human parainfluenza virus type 2; human metapneuomovirus; and enteroviruses.
49. The method of clause 47, wherein the infectious agent is a microorganism selected from the group consisting of Rickettsia, Chlamydia, Mycobacteria, Clostridia, Corynebacteria, Mycoplasma, Ureaplasma, Legionella, Shi-gella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., and Helicobacter spp.
50. The method of clause 47, wherein the infectious agent is a microorganism selected from the group consisting of Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum. Bacillus anthracis, Salmonella typhi, Vibrio cholera. Pasteurella pestis (Yersinia pestis), Pseudomonas aeruginosa, Campylobacter jejuni, Clostridium dificile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.
51. The method of clause 45, wherein the disorder comprises an autoimmune disease selected from the group consisting of diabetes, cutaneous psoriasis, hyperthyroidism, autoimmune adrenal insufficiency, hemolytic anemia, rheumatic carditis, systemic lupus erythematosus, cutaneous lupus erythematosis, psoriatic arthritis, Sjogren's syndrome polymyositis, myositis, multiple sclerosis, Crohn's disease, ulcerative colitis, lupus, inflammatory bowel syndrome, dermatomyositis, dermatitis, atopic dermatitis, scleroderma, polymyositis, psoriasis, alopecia areata, rheumatoid arthritis, graft-versus-host disease, interferonopathies, irritable bowel syndrome, and a combination of two or more of these.
52. The method of clause 45, wherein the disorder comprises an inflammatory condition, the inflammatory condition comprising acute kidney injury.
53. The method of clause 45, wherein the disorder comprises a respiratory condition, the respiratory condition comprising asthma, lung fibrosis, or a combination of asthma and lung fibrosis.
54. The method of clause 45, wherein the disorder comprises a skin disorder selected from the group consisting of scleroderma, psoriasis, dermatomyosititis, atopic dermatitis, and a combination of two or more of these.
55. The method of clause 45, wherein the disorder comprises a cancer selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, B-cell acute lymphocytic leukemia blastic plasmacytoid dendritic cell neoplasm (“BPDCN” or “pDC leukemia”), chronic lymphocytic leukemia, acute myelogenous leukemia, chronic myelogenous leukemia (“AML”), chronic granulocytic leukemia, acute granulocytic leukemia, aging-related hairy cell leukemia, chronic myelomonocytic leukemia (“CMML”), myelodysplastic syndrome, chronic myelofibrosis, multiple myeloma, neuroblastoma, rhabdomyosarcoma, Kaposi's sarcoma, Waldenström's macroglobulinemia, diffuse large B-cell lymphoma (DLBCL) (L265P), polycythemia vera, essential thrombocytosis, Hodgkin's disease, non-Hodgkin's lymphoma, soft-tissue sarcoma, osteogenic sarcoma, primary macroglobulinemia, retinoblastoma, and a combination of two or more of these.
56. The method of clause 44 to 55, wherein the epitope comprises an amino acid sequence given by SEQ ID NO: 11 and the antibody or fragment thereof binds the epitope with an affinity (K_{d}) from 0.1 nanomolar (nM) 100 nM.
57. The method of clause 44 to 56, wherein the antibody or fragment thereof is a monoclonal antibody or fragment thereof.
58. The method of clause 44 to 57, wherein the antibody or fragment thereof is a humanized antibody or fragment thereof.
59. The method of clause 44 to 58, wherein:
   the heavy chain variable region comprises an amino acid sequence given by SEQ ID NO: 1 or a sequence at least about 90% identical to SEQ ID NO: 1; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
60. The method of clause 44 to 58, wherein:
   the heavy chain variable region comprises an amino acid sequence given by SEQ ID NO: 2 or a sequence at least about 90% identical to SEQ ID NO: 2; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
61. The method of clause 44 to 58, wherein:
   the heavy chain variable region comprises an amino acid sequence given by SEQ ID NO: 3 or a sequence at least about 90% identical to SEQ ID NO: 3; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
62. The method of clause 44 to 58, wherein:
   the heavy chain variable region comprises an amino acid sequence given by SEQ ID NO: 4 or a sequence at least about 90% identical to SEQ ID NO: 4; and
   the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
63. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 5 or a sequence at least about 90% identical to SEQ ID NO: 5.
64. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 6 or a sequence at least about 90% identical to SEQ ID NO: 6.
65. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 7 or a sequence at least about 90% identical to SEQ ID NO: 7.
66. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 8 or a sequence at least about 90% identical to SEQ ID NO: 8.
67. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 9 or a sequence at least about 90% identical to SEQ ID NO: 9.
68. The method of clause 44 to 58, wherein:
   the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   the light chain variable region comprises an amino acid sequence given by SEQ ID NO: 10 or a sequence at least about 90% identical to SEQ ID NO: 10.
69. The method of clause 44 to 59 or 63, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 5 or a sequence at least about 90% identical to SEQ ID NO: 5.
70. The method of clause 44 to 58, 60, or 63, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 5 or a sequence at least about 90% identical to SEQ ID NO: 5.
71. The method of clause 44 to 58, 61, or 63, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 5 or a sequence at least about 90% identical to SEQ ID NO: 5.
72. The method of clause 44 to 58, 62, or 63, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 5 or a sequence at least about 90% identical to SEQ ID NO: 5.
73. The method of clause 44 to 59 or 64, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 6 or a sequence at least about 90% identical to SEQ ID NO: 6.
74. The method of clause 44 to 59 or 65, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 7 or a sequence at least about 90% identical to SEQ ID NO: 7.
75. The method of clause 44 to 59 or 66, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 8 or a sequence at least about 90% identical to SEQ ID NO: 8.
76. The method of clause 44 to 59 or 67, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 9 or a sequence at least about 90% identical to SEQ ID NO: 9.
77. The method of clause 44 to 59 or 68, wherein:
   the heavy chain variable region comprises an amino acid sequence given by SEQ ID NO: 1 or a sequence at least about 90% identical to SEQ ID NO: 1; and
   the light chain variable region comprises an amino acid sequence given by SEQ ID NO: 10 or a sequence at least about 90% identical to SEQ ID NO: 10.
78. The method of clause 44 to 58, 60, or 64, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 6 or a sequence at least about 90% identical to SEQ ID NO: 6.
79. The method of clause 44 to 58, 61, or 64, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 6 or a sequence at least about 90% identical to SEQ ID NO: 6.
80. The method of clause 44 to 58, 62, or 64, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 6 or a sequence at least about 90% identical to SEQ ID NO: 6.
81. The method of clause 44 to 58, 60, or 65, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 7 or a sequence at least about 90% identical to SEQ ID NO: 7.
82. The method of clause 44 to 58, 61, or 65, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 7 or a sequence at least about 90% identical to SEQ ID NO: 7.
83. The method of clause 44 to 58, 62, or 65, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 7 or a sequence at least about 90% identical to SEQ ID NO: 7.
84. The method of clause 44 to 58, 60, or 66, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 8 or a sequence at least about 90% identical to SEQ ID NO: 8.
85. The method of clause 44 to 58, 61, or 66, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 8 or a sequence at least about 90% identical to SEQ ID NO: 8.
86. The method of clause 44 to 58, 62, or 66, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 8 or a sequence at least about 90% identical to SEQ ID NO: 8.
87. The method of clause 44 to 58, 60, or 67, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 9 or a sequence at least about 90% identical to SEQ ID NO: 9.
88. The method of clause 44 to 58, 61, or 67, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 9 or a sequence at least about 90% identical to SEQ ID NO: 9.
89. The method of clause 44 to 58, 62, or 67, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 9 or a sequence at least about 90% identical to SEQ ID NO: 9.
90. The method of clause 44 to 58, 60, or 68, wherein:
   the heavy chain variable region comprises an amino acid sequence given by SEQ ID NO: 2 or a sequence at least about 90% identical to SEQ ID NO: 2; and
   the light chain variable region comprises an amino acid sequence given by SEQ ID NO: 10 or a sequence at least about 90% identical to SEQ ID NO: 10.
91. The method of clause 44 to 58, 61, or 68, wherein:
   the heavy chain variable region comprises an amino acid sequence given by SEQ ID NO: 3 or a sequence at least about 90% identical to SEQ ID NO: 3; and
   the light chain variable region comprises an amino acid sequence given by SEQ ID NO: 10 or a sequence at least about 90% identical to SEQ ID NO: 10.
92. The method of clause 44 to 58, 62, or 68, wherein:
   the heavy chain variable region comprises an amino acid sequence given by SEQ ID NO: 4 or a sequence at least about 90% identical to SEQ ID NO: 4; and
   the light chain variable region comprises an amino acid sequence given by SEQ ID NO: 10 or a sequence at least about 90% identical to SEQ ID NO: 10.
93. A kit for depleting, inhibiting, or depleting and inhibiting plasmacytoid dendritic cells (pDC) in a patient in need thereof, said kit comprising:
instructions; and
an antibody or fragment thereof comprising:
   a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
94. A pharmaceutical composition comprising:
at least one excipient; and
an antibody or fragment thereof comprising:
   a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
   a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.
95. An antibody or fragment thereof according to clause 1 for use in therapy.
96. An antibody or fragment thereof according to clause 1 for use in depleting, inhibiting, or depleting and inhibiting plasmacytoid dendritic cells (pDC).
97. The use of an antibody or fragment thereof according to clause 1 for the manufacture of a medicament.

### EXAMPLES

Examples related to the present disclosure are described below. In most cases, alternative techniques can be used. The examples are intended to be illustrative and are not limiting or restrictive of the scope of the invention as set forth in the claims.

To define the role of and elucidate the immunologic mechanisms of tumor-associated/TA-pDC in the human breast cancer tumor microenvironment (“BC-TME”) that contribute to immune suppression, and to develop novel therapeutics (combining pDC depleting antibody with other immunotherapeutics), a fully humanized antibody (NPB-303) that specifically binds human BDCA2 and inhibits/depletes human pDC in vitro and in vivo was developed. In a humanized mouse model with human immune and human BC tumor cells (“BC-HIT mice”), it was demonstrated that TA-pDC in the BC-TME play a role in the immune suppressive TME and depletion of TA-pDC in BC-TME reversed immune suppression to control human BC growth in vivo. These findings may be used as a basis of a clinical study to test whether TA-pDC depletion will show enhanced antitumor activity with or without other immune-oncology (“IO”) drugs in BC patients. The mechanism of TA-pDC in programing the BC-TME may be investigated, and novel immune therapeutics in human BC-HIT mice may be modeled.

### Example 1

### Humanization of Mouse Monoclonal Antibody 12b

The cDNA sequences for mouse monoclonal antibody 12b heavy chain variable (“VH”) regions and light chain variable (“VL”) regions were determined. These sequences were used to design optimized VH region and VL region expression constructs, where codons rarely used in mammals were replaced with frequently used alternatives. These gene constructs were synthesized and cloned into the human IgG1 antibody expression vector. VH region and VL region genes were cloned upstream of either the heavy chain constant regions (CH1, hinge, CH2 and CH3), or light chain constant regions (VL/kappa), as appropriate. The resulting chimeric antibody was designated NPB-301.

A three-dimensional molecular model of the mouse 12b variable regions was constructed using JN Biosciences' proprietary algorithm. This was used to identify framework amino acid residues implicated in the formation of CDR structures. In parallel, the mouse VH and VL domains from 12b were searched within the GenBank® database to identify highly homologous human VH and VL domains, which were selected as acceptors for CDR grafting. Finally, gene constructs were designed where 12b CDRs were grafted onto the human acceptor frameworks (i.e. original human CDRs were replaced), and human framework residues corresponding to various structural positions in 12b were back-mutated to the mouse sequence to ensure functional CDR orientation.

Genes encoding the humanized VH and VL domains were cloned into an IgG1 expression vector as described above for chimeric antibodies. The resulting humanized antibody variants were designated as NPB-30X, with different humanized variants indicated by a different final number (i.e. NPB-302, NPB-303 etc.).

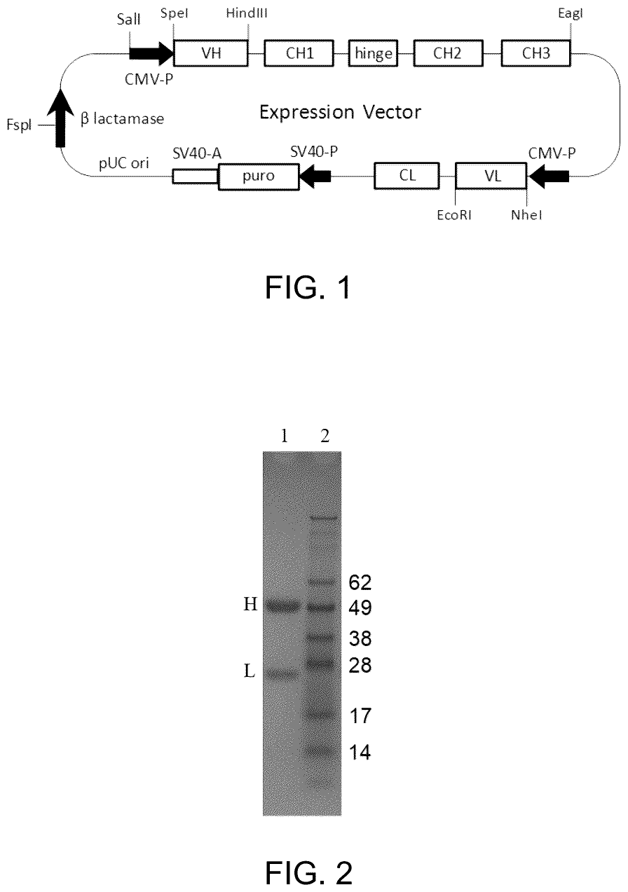

FIG. 1 provides a schematic diagram of the expression vectors used to produce chimeric and humanized anti-BDCA2 antibodies. The plasmid contains the heavy chain transcription unit starting with the human CMV promoter (CMV-P) to initiate transcription of the antibody heavy chain gene. The CMV promoter is followed by the VH, CH1, hinge, CH2, and CH3 exons. After the heavy chain gene sequence, the light chain transcription unit begins with the CMV promoter, followed by the VL exon and CL (human kappa chain constant region) exon. The light chain gene is then followed by the SV40 early promoter (SV40-P), the puromycin N-acetyl-transferase gene (puro) for resistance to puromycin, and a segment containing the SV40 polyadenylation site (SV40-A). Finally, the plasmid contains a part of the plasmid pUC19, comprising the bacterial origin of replication (pUC ori) and beta-lactamase gene (P lactamase). Arrows indicate the orientation of transcription. Relevant restriction enzyme sites are also shown in the FIG. 1.

To obtain cell lines for stable antibody production, the expression vectors of FIG. 1 were stably transfected into CHO-K1 cells (ATCC). Briefly, CHO-K1 cells were cultured in SFM4CHO media (HyClone) at 37° C. and 7.5% CO₂. For transfection, 20 μg of the expression vector was linearized using the FspI restriction enzyme and transferred into approximately 107 cells by electroporation. Transfected cells were diluted in SFM4CHO media and aliquoted across several 96-well plates. After 48 hrs, 10 μg/ml of puromycin was added to select for stable integrants. Cells were kept under puromycin selection for 10 days.

Antibody expression was measured by ELISA. Briefly, plates were coated with a human IgG Fcγ-chain-specific polyclonal antibody (Sigma-Aldrich). Wells were then blocked with PBS containing 0.05% Tween20 and 2% milk. Wells were washed and 100 μL of supernatant from each stable cell line. Supernatant was added and incubated for 60 mins. Supernatants were removed by washing, and a HRP-conjugated goat anti-human kappa chain polyclonal antibody (Southern Biotech) was added and incubated for 30 mins. After a final wash, the ELISA signal was developed by adding ABTS substrate and quenched with 2% oxalic acid.

The assay signals were analyzed at 405 nM using a plate reader. All antibodies were diluted in PBS with 0.1% skim milk and 0.025% Tween 20. Wash steps using PBS-T (PBS with 0.05% Tween20) were applied between each of the incubation and signal development steps.

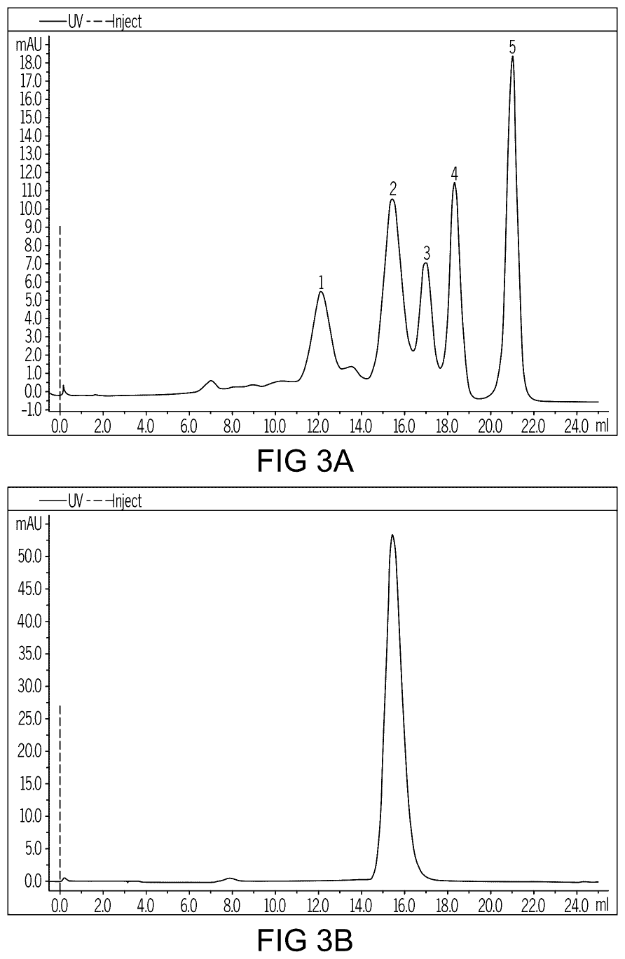

Stable transfectants were grown in SFM4CHO medium using roller bottles until the cell viability was under 50%. Supernatants were harvested and clarified by centrifugation and filtration. Samples were loaded on a protein-A Sepharose column (HiTrap MabSelect Xtra, GE Healthcare). The column was washed with PBS and captured antibodies were eluted with 0.1 M glycine-HCl (pH 3). The eluted antibodies were neutralized with 1 M Tris-HCl (pH 8) and buffer-exchanged into PBS by dialysis. Antibody concentration was determined by measuring absorbance at 280 nm (1 mg/ml=1.4 OD). Purified NPB-301, NPB-302, and NPB-303 were characterized by SDS-PAGE under reducing conditions according to standard procedures. FIG. 2 provides the SDS-PAGE analysis of NPB-303. FIG. 3 provides the protein analysis of NPB-303 on a Superose 6 column using an AKTA FPLC system. Panel A provides the results with a gel filtration standard produced by BioRad. Panel B provides the results with NPB-303. This preparation had a greater than 98% antibody monomer, which has a molecular weight of 155 kD.

Production of recombinant human and rhesus BDCA2 proteins, and measurement of their binding affinity to the anti-BDCA2 antibodies by surface plasmon resonance (SPR) experiments, was conducted by Mass Biologics. The extracellular domain of either human BDCA2 (UniProt Q8WTTO, residues 45-213) or rhesus BDCA2 (UniProt A0A1D5QFY5, residues 45-212) were cloned into a Mass Biologics' pYW7 vector, or pcDNA3.4, respectively. Proteins were expressed using the ExpiCHO expression system (Thermo Fisher Scientific). Purification was conducted by affinity chromatography using a Ni Sepharose Excel column (GE Life Sciences), followed by a standard size exclusion chromatography step to remove aggregates and degradation products.

BDCA2 antibody binding kinetics were measured using a Biacore surface plasmon resonance (SPR) instrument (GE Life Sciences). Briefly, anti-BDCA2 antibodies were captured on a Protein A sensor chip (GE Life Sciences). The recombinant BDCA2 proteins were then flowed over the captured antibodies over a range of concentrations, at a rate of 50 μL/minute. The data was analyzed to determine kinetic constants using the BIAevaluation software package (GE Life Sciences).

NPB-303 was selected for further study. The heavy chain sequence and light chain sequence for NPB-303 are given by SEQ ID NO: 13 and SEQ ID NO: 14, respectively.

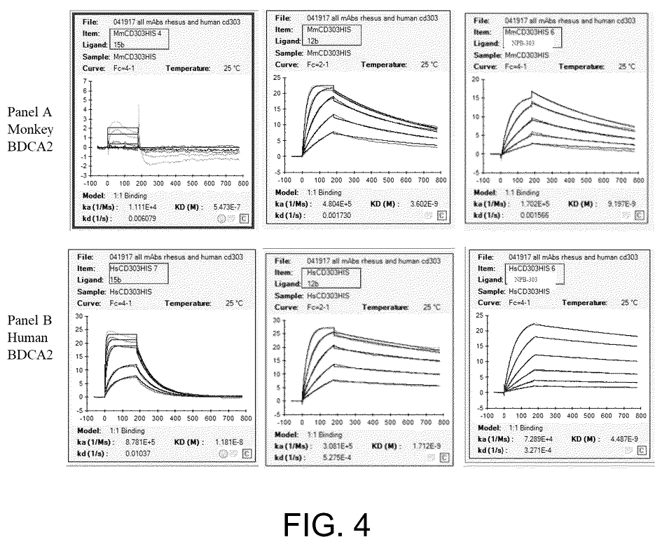

FIG. 4 shows the biophysical binding of 15b, 12b, and NPB-303 to BDCA2 antigens from either monkey (Panel A) or human (Panel B). The binding data are also summarized in Table 1. In Table 1, monoclonal antibody (mAb) 15b is a mouse monoclonal antibody that is human BDCA2-specific, mAb 12b is the mouse monoclonal antibody that was humanized to produce NPB-303 that binds both human and monkey BDCA2, and NPB-303 is the humanized monoclonal antibody.

TABLE 1

| Species | mAb | kₐ (Ms⁻¹) | k_{d} (s⁻¹) | KD (M) | rₘₐₓ |
|---|---|---|---|---|---|
| Monkey | 15b | Did not bind | | | |
| BDCA2 | 12b | 4.8 × 10⁵ | 1.73 × 10⁻³ | 3.6 × 10⁻⁹ | 21.8 |
| | NPB-303 | 1.7 × 10⁵ | 1.57 × 10⁻³ | 9.2 × 10⁻⁹ | 18.5 |
| Human | 15b | 8.78 × 10⁵ | 1.04 × 10⁻² | 1.18 × 10⁻⁸ | 20.1 |
| BDCA2 | 12b | 3.08 × 10⁵ | 5.28 × 10⁻⁴ | 1.71 × 10⁻⁹ | 25.9 |
| | NPB-303 | 7.29 × 10⁵ | 3.27 × 10⁻⁴ | 4.49 × 10⁻⁹ | 23.3 |

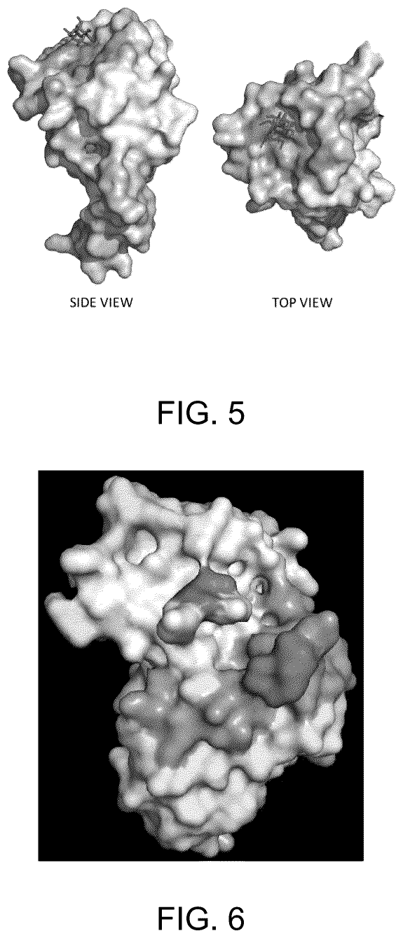

FIG. 5 is a structural model of the BDCA2 extracellular domain (green). The yellow-shaded region is the NPB-303 epitope, and the orange-shaded region are the BDCA2 lectin (ligand) binding residues. FIG. 6 is a structural model of the NPB-303 variable (binding) domain. In the figure, the VH framework is shown in green; the VH CDRs are shown in yellow; the VL framework is shown in blue; the VL CDRs are shown in brown, and aspartic acid isomerization motifs are shown in red.

### Example 2

### In Vitro and In Vivo Binding Characterization of NPB-303 Antibodies

An ELISA assay for NPB-303 binding was developed in-house. Briefly, Nunc MaxiSorp 96-well plates were coated with BDCA-2 antigen (Biorbyt CLEC4C orb358925) overnight at 4° C. After washing, the plates were blocked by the Blocking Buffer (1% BSA in 1×PBS). The diluted standard NPB-303 antibodies and other test lots were added to the plates. After 1 hour incubation at room temperature, the plates were washed and added with the secondary antibody (HIRP-conjugated Goat anti-Human IgG Fc antibody; ThermoFisher A18829) for 1 hour at RT. The signals were developed by adding TMB substrate reagent (ThermoFisher 34021). The signals at 450 nm and 590 nm were read on the plate reader.

NPB-303 antibody and human IgG control antibody were conjugated with the APCCy7 label using LIGHTNING-LINK™ Antibody Labeling Kits (Novus Biologicals, Cat #765-0005) following the instruction within the kit. One million fresh or frozen human PBMCs were stained with Live/Dead™ Dye (Aqua Yellow), and either NPB-303-APCCy7 or Human IgG-APCCy7 were added, along with human CD45-BV786, CD3/CD14/CD19-FITC, CD4-PacBlue, CD123-PE, and BDCA2-APC. Cells were acquired on a BD FACSCelesta and analyzed using a FlowJo program.

Umbilical cord blood was obtained from the National Disease Research Interchange (NDRI, Philadelphia, PA), and the use of cord blood was exempted by New England Institutional Review Board (IRB). Human hematopoietic stem cells (HSCs) were enriched with the use of ROSETTESEP™ Human CD3 Depletion Cocktail (Stem Cell Technologies). The cells were stained with Viability eF780 (BioLegend), FITC CD3 (BioLegend), PE CD34 (BioLegend), APC CD133/2 (Miltenyi), and analyzed on a BD Accuri Flow Cytometer. Viable cells were counted by ViCell (Beckman Coulter). All cells were isolated under sterile conditions. To construct humanized mice, 1-5×105 CD34+CD133+ cord blood cells with the addition of 1 μg/mL OKT3 mAb were injected into 4-6-week-old pre-irradiated mice intravenously. Ten to twelve weeks after engraftment, mice were analyzed for reconstitution by flow cytometry analysis of peripheral blood. Mice that had 20% or more human leukocyte reconstitution were used in subsequent experiments.

All experiments involving animals were approved by Institutional Animal Care & Use Committee (IACUC) at NPB. Female immunodeficiency mice including NSG mice (NOD.Cg-Prkdc^{scid} Il2rg^{tmlWjl}/SzJ) and NBSGW (NOD.Cg-Kit^{W-41J} Tyr⁺ Prkdc^{scid} Il2rg^{tmlWjl}/ThomJ) from Jackson Laboratory and NP1 (NOD-Rag1−/−IL2RgammaC-null) mice from NPB-USA were used for the construction of humanized mice. The NSG mice and NP1 mice were irradiated (180 rads for NSG mice and 400 rads for NP1 mice using Xrad IR225 X-ray Irradiator) at least 4 hours before the reconstitution. Some experiments were conducted by using humanized NSG mice (1HCM-Humanized CD34 cohort, >60% engraftment) purchased from Jackson Laboratory.

Humanized mice were injected with NPB-303 (10 mg/kg IV) at day 0 and day 2. Mice were sacrificed at Day 3. Cells from blood were stained with Live/Dead™ Fixable Yellow (Cat#L34959, Thermo Fisher), human CD45-BV786, mouse CD45.1-APCCy7, human CD3/CD14/CD19-FITC, human CD4-PacBlue, CD123-PE, and BDCA2-APC (all from BioLegend) and analyzed using a FlowJo program.

Human PBMCs were incubated with NPB-303 antibody or human IgG1 isotype control mAb (10 μg/mL) for 30 min at 37° C. The PBMCs were then stimulated with CpG-ODN-2216 (Invivogen, Catalog #tlrl-2216, 2 μg/mL) for 16 hrs. Cells were treated with GolgiPlug for 3 hours before Flow Cytometry analysis. After cultivation, human PBMCs were stained with surface markers including Live-Dead Yellow Dye, human CD45-BV786, CD3-FITC, CD14-FITC, CD19-FITC, CD123-PECy7, BDCA2-APC, CD4-Pacific Blue, followed by intracellular staining for IFNα-PE or IgG1-PE isotype control using Fixation/Permeabilization solution (BD Cytofix/Cytoperm, Cat #: 54714). Stained cells were analyzed using a BD Celesta Flow Cytometer, and data were analyzed using FlowJo software.

Human PBMCs were plated at 1×10⁶/well in complete RPMI media (10% FBS, 1× non-essential amino acids, 1×Pen-Strp, 1 mM sodium pyruvate, 10 mM HEPES, 50 μM 2-mercaptoethanol and 2 mM L-Glutamine) in 96-well round-bottom plates and treated with 10, 3.33, 1.11, 0.37, 0.124, 0.04, 0.014, 0.005, 0.0015 and 0.0005 μg/mL of NPB-303 antibody or 10 ρg/mL of an isotype control antibody for 1 hour. All conditions were established in duplicate. Cells were then stimulated with a final concentration of 10 mg/mL of CpG-ODN-2216 and cultured for 16 h at 37° C. and 5% C02. The supernatants were collected and IFNα was evaluated using PBL Human IFN Alpha Multi-Subtype ELISA Kit (Serum, Plasma, TCM). IC₅₀ was calculated using GraphPad Prism program.

Human PBMCs were incubated with NBP-303 antibody or human IgG1 isotype control mAb (10 μg/mL) for 30 min at 37° C. The PBMCs were then stimulated with CpG-ODN-2216 (Invivogen, Catalog #tlrl-2216, 2 μg/mL) for 16 hrs. Cells were treated with GolgiPlug for 3 hours before Flow Cytometry analysis. After cultivation, human PBMCs were stained with surface markers including Live-Dead Yellow Dye, human CD45-BV786, CD3-FITC, CD14-FITC, CD19-FITC, CD123-PECy7, BDCA2-APC, CD4-Pacific Blue, followed by intracellular staining for IFNα-PE or IgG1-PE isotype control using Fixation/Permeabilization solution (BD Cytofix/Cytoperm, Cat #: 54714). Stained cells were analyzed using a BD Celesta Flow Cytometer, and data were analyzed using FlowJo software.

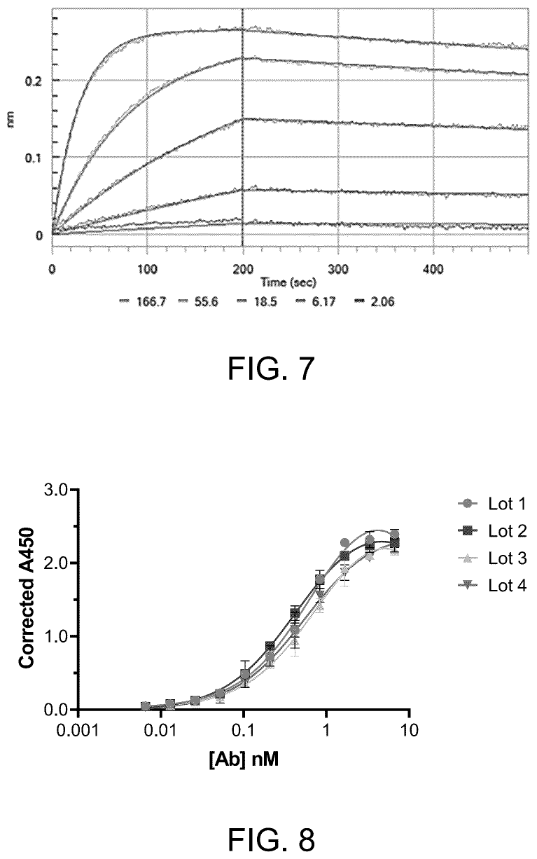

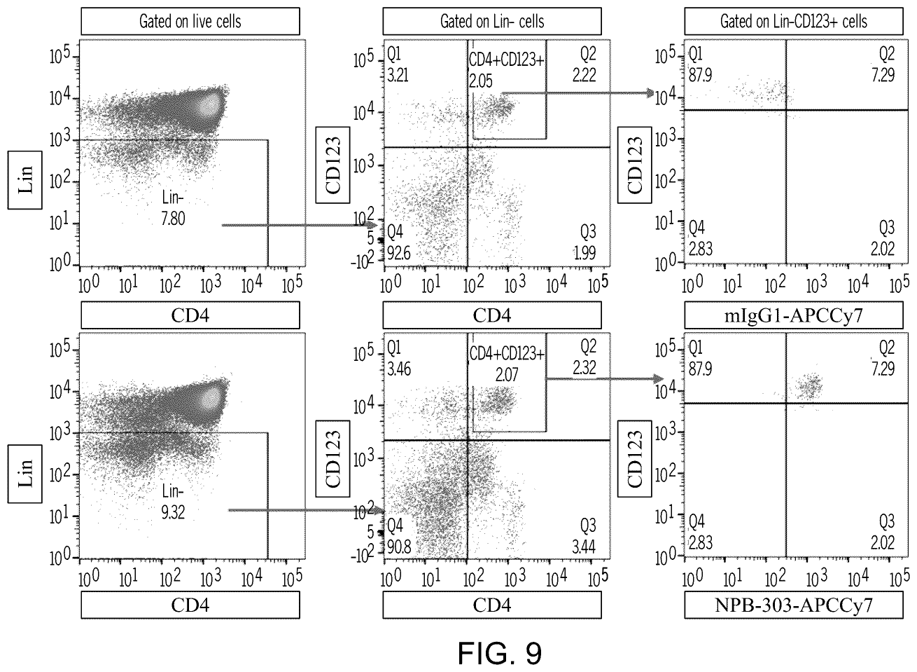

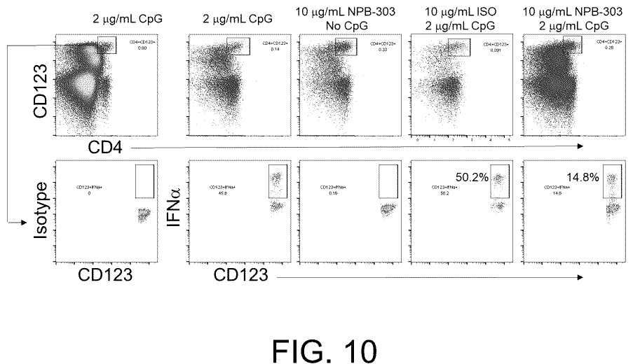

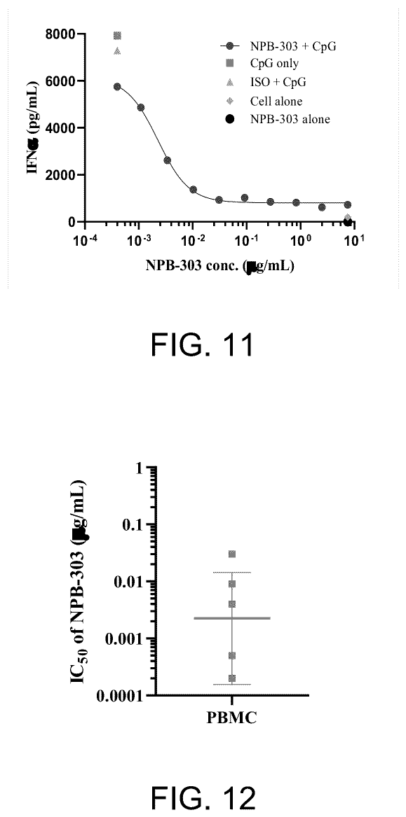

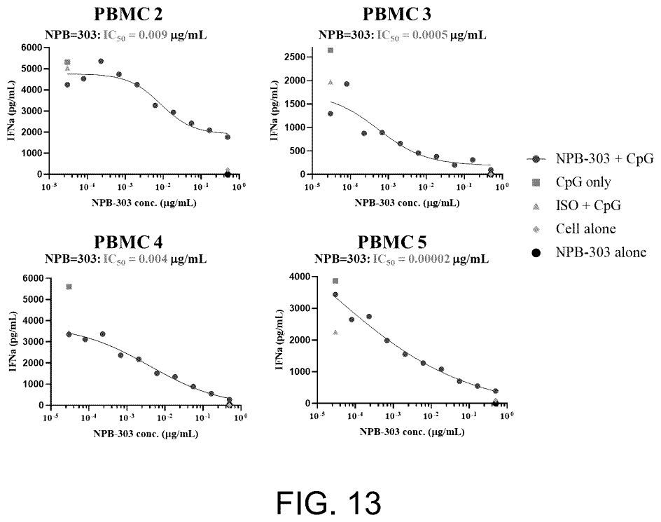

NPB-303 binds BDCA2 on human pDCs (in vitro) and depletes/inhibits pDCs in vivo. To elucidate the role of the chronic pDC/IFN-I axis in suppression of anti-tumor immunity in the tumor microenvironment, a novel humanized antibody was developed to target human pDC for treating human inflammatory diseases associated with TA-pDC. A mouse monoclonal antibody that specifically binds to human BDCA2 on pDC was engineered to replace the Fc domain and non-antigen binding regions of Fab domains to human antibody sequences (see FIG. 1). The NPB-303 antibody (huIgG1) showed highly stable interaction (FIG. 7) with BDCA2 proteins (FIG. 8, K_{d}˜1 nM, kₒₙ(1/ms)˜2.1×10⁵ and k_{off} (1/s)˜3.3×10⁴). NPB-303 specifically binds to human pDC in vitro (FIG. 9). The BDCA2-mediated suppression of pDC activation in vitro of NPB-303 was examined. NPB303 effectively inhibited pDC activation by CpG, as demonstrated by intracellular IFNα detection (FIG. 10), or by detection of IFNα in the supernatant (FIG. 11). With similar study in multiple human donor PBMC samples, the IC₅₀ was estimated to be −3 ng/ml (FIG. 12 and FIG. 13), significantly better than the recently reported Biogen BDCA2 Mab.

### Example 3

### Pharmacokinetic and Pharmcodynamic Characterization of NPB-303 Antibodies

All animals used were NOD Rag Gamma (NRG) mice purchased from The Jackson Laboratory (cat #: 007799). Animals were all male and 5-6 weeks of age. Animals were injected with NPB-303 at a concentration of 10 mg/mL. The dosing volume was 10 mL/kg, resulting in an antibody dose of 10 mg/kg. Mice were dosed once at the start of the experiment via either IV (tail vein) or SC (flank). Following NPB-303 injection, blood was collected at various time points via cheek bleeds. A group of three animals was treated for each time point. In-life samples were limited to 50 μL per bleed, with an overall limit of ˜200 μL (10% blood volume) over the two-week study period. Prior to the final sample taken from a given animal, the mouse was euthanized by CO₂ asphyxia and a terminal blood draw was collected by cardiac puncture. Blood was collected in K₃EDTA tubes (Greiner Bio-One) and processed into plasma.

NPB-303 in plasma samples was detected using an IgG (Total) Human Uncoated ELISA Kit (ThermoFisher cat #: 88-50550-22) according to the manufacturer's instructions. A pilot optimization was performed using 16 samples (n=1) at 5 different dilutions: 1:50, 1:2000, 1:500, 1:5000 and 1:10,000. The samples tested also included a standard curve constructed by preparing a solution of purified NPB-303 at 100 ng/mL, which was then used to produce 2-fold serial dilutions (7 concentrations in total, ranging from ˜1.6-100 ng/mL). The NPB-303 concentration in plasma samples was estimated by comparison with this curve. Data was used to calculate half-life (T_{1/2}), maximum plasma concentration (Cₘₐₓ), time to reach maximum plasma concentration (Tₘₐₓ), mean residence time (MRT), area under the plasma concentration curve (AUC), apparent total body clearance (CL) and the apparent volume of distribution during the terminal phase (VZ). All data analysis was conducted using the WinNonlin 7.0 software package (Certara).

The NPB-303 was further characterized to quantify endotoxin present.

Humanized female mice were injected with NPB-303 at 10 mg/kg i.v. Mice were sacrificed at the time points of 0, 1, 6, 24, 48, 72 and 168 hours after injection. Cells from mouse spleen were stained with Live/Dead Dye, mouse CD45.1 APCCy7 and human antibodies including CD3 FITC, CD14 FITC, CD19 FITC, CD123 PE, BDCA2 APC, CD4 Pacific Blue, CD45 BV786 (all from BioLegend). Cells were analyzed on a BD Celesta Instrument and data was analyzed using FlowJo software.

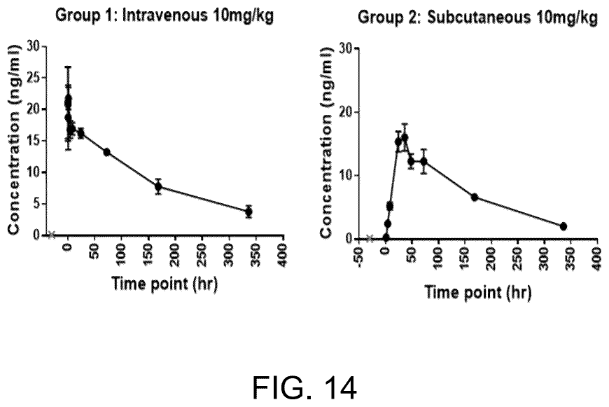

The pharmacokinetics of NPB-303 in NSG mice were investigated by iv or sc routes (FIG. 14 and Table 2). In Table 2, T_{1/2} is the terminal half-life of the analyte in plasma, Tₘₐₓ is the time from dosing to a maximum measured concentration, Cₘₐₓ is the maximum measured concentration of the analyte in plasma, MRT is the mean residence time, AUC_{0-∞} is the area under the concentration-time curve of the analyte in plasma over the time interval from 0 extrapolated to infinity, CL is the clearance, and Vz is the apparent volume of distribution during the terminal phase. In FIG. 14, the crosses on the “time point (hr)” axis indicate the plasma concentration of NPB-303 detected in samples taken 30 minutes prior to antibody injection. The estimated half-life of NPB-303 was 6.2 days and 4.2 days respectively for iv and sc routes. The Tₘₐₓ was 1 hr and 36 hr respectively, and Cₘₐₓ was 109 μg/ml and 80 μg/ml, respectively, for iv and sc routes. No adverse effect was detected. Therefore, the value calculated for these parameters and provided in Table 2 is highly dependent on the sampling protocol.

TABLE 2

| Parameter | IV | SC |
|---|---|---|
| T_{1/2} (h) | 149 (6.2 days) | 101 (4.2 days) |
| Tₘₐₓ (h) | 1 | 36 |
| Cₘₐₓ (μg/ml) | 109 | 80 |
| MRT (h) | 117 (4.9 days) | 113 (4.7 days) |
| AUC_{0-∞} (mg · hr/ml) | 19.6 | 13.8 |
| Clearance (l/day/kg) | 0.012 | 0.017 |
| V_{Z} (l/kg) | 0.1 | 0.1 |

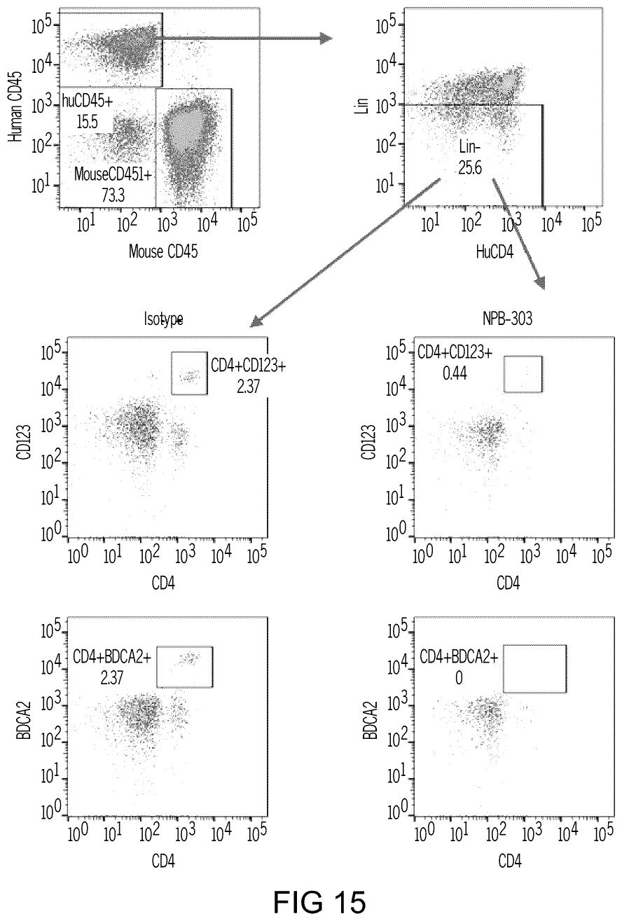

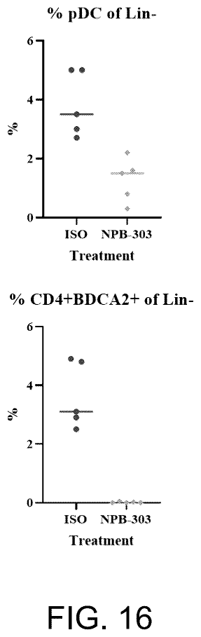

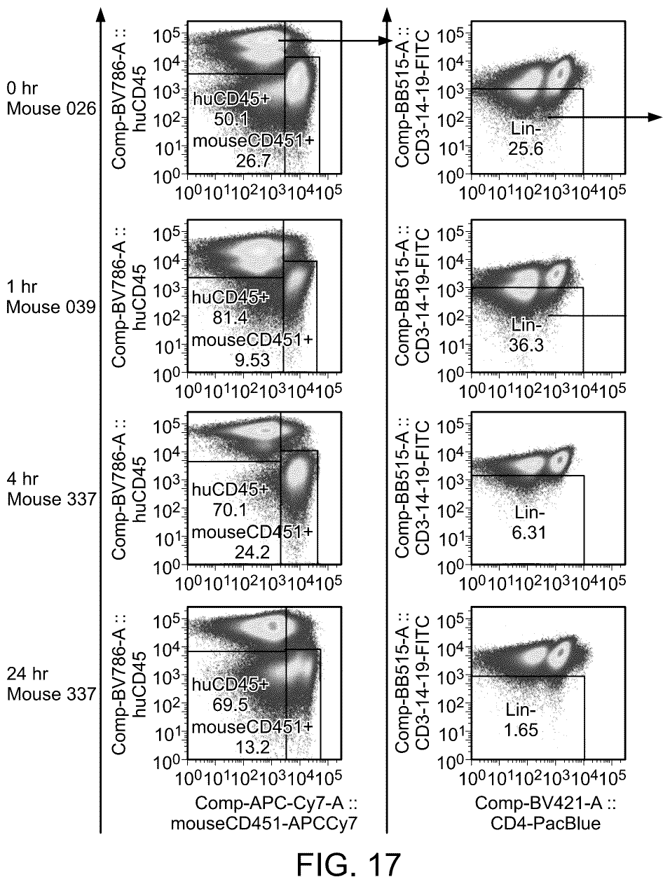

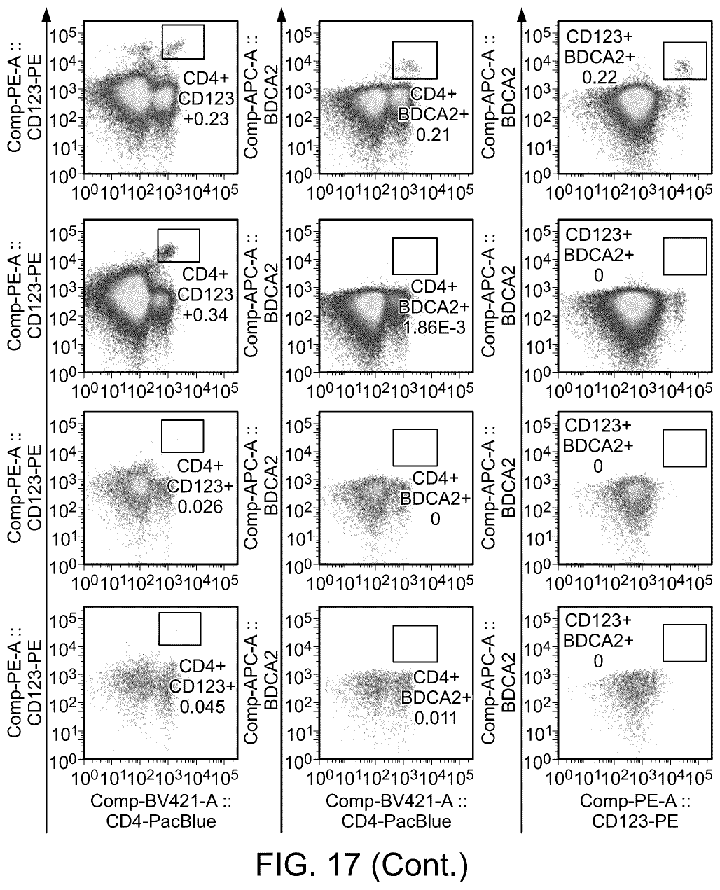

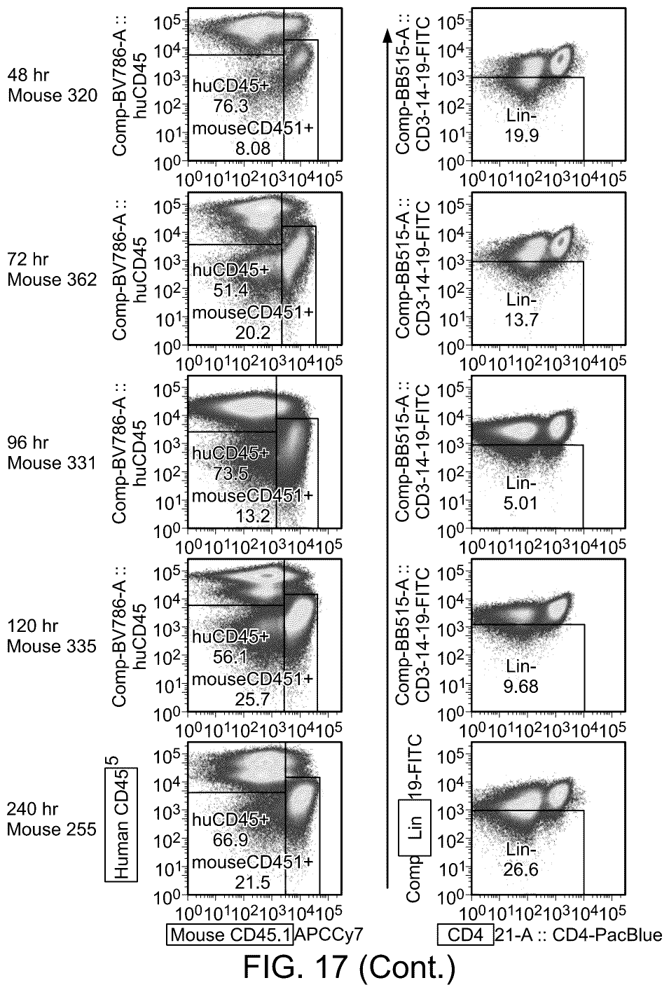

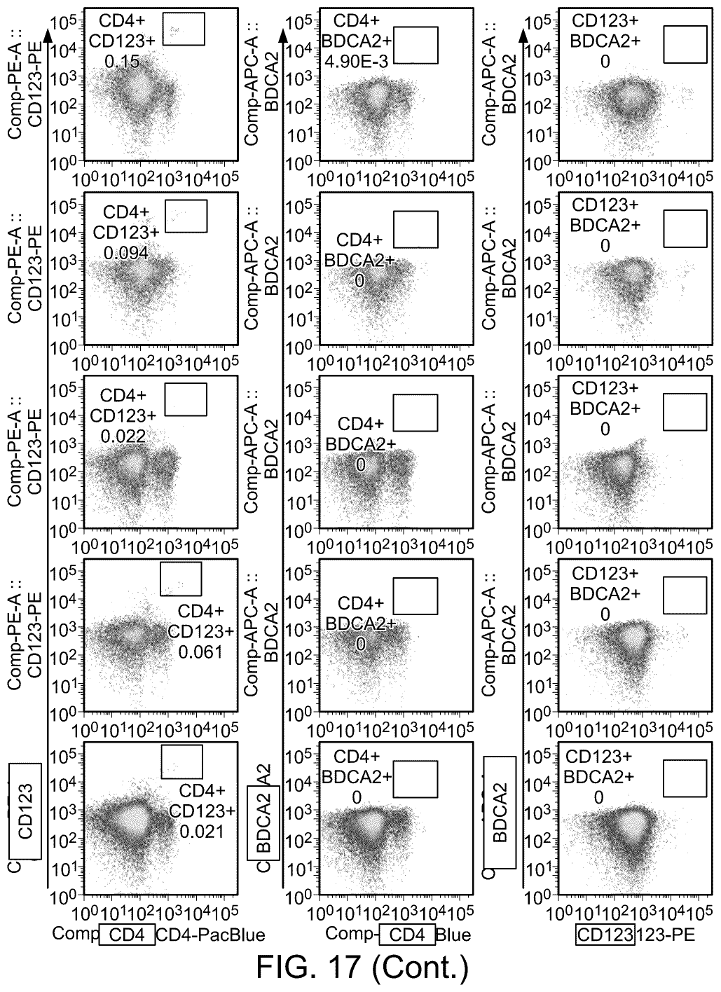

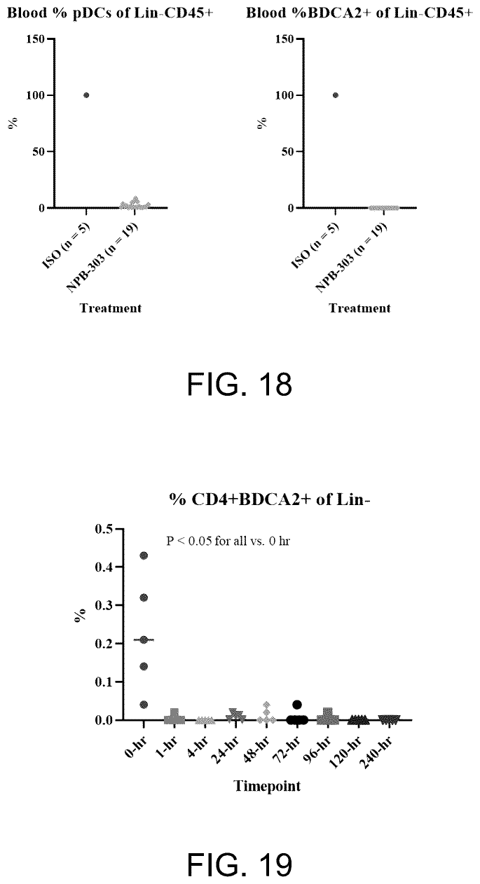

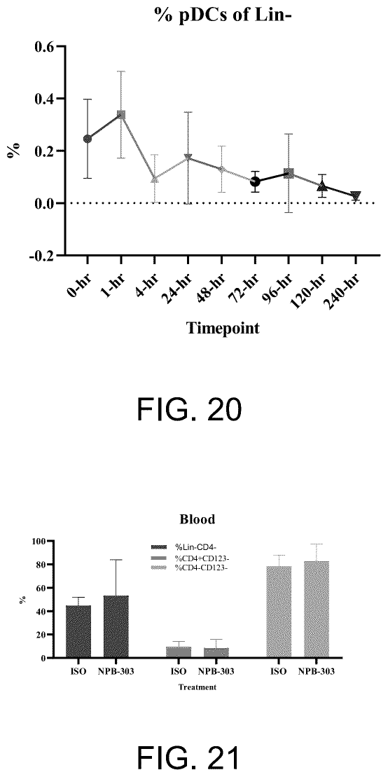

When tested in mice engrafted with human CD34+ HSC/HSPC, NPB-303 efficiently and specifically depleted human pDC in humanized mice in vivo (FIG. 15, FIG. 16, FIG. 17, and FIG. 18). The BDCA2 surface protein was efficiently internalized on the remaining pDC on 10 days after mAb treatment (FIG. 19). Human pDC in lymphoid organs were efficiently depleted and/or inhibited by BDCA2 binding (FIG. 19 and FIG. 20). The humanized NPB-303 mAb thus specifically binds to BDCA2 on human pDC and efficiently inhibits and depletes pDC in vitro and in vivo. However, NPB-303 did not deplete other cell types (Lin−CD4−, CD4+CD123− and CD4−CD123−) in blood, as shown in FIG. 21.

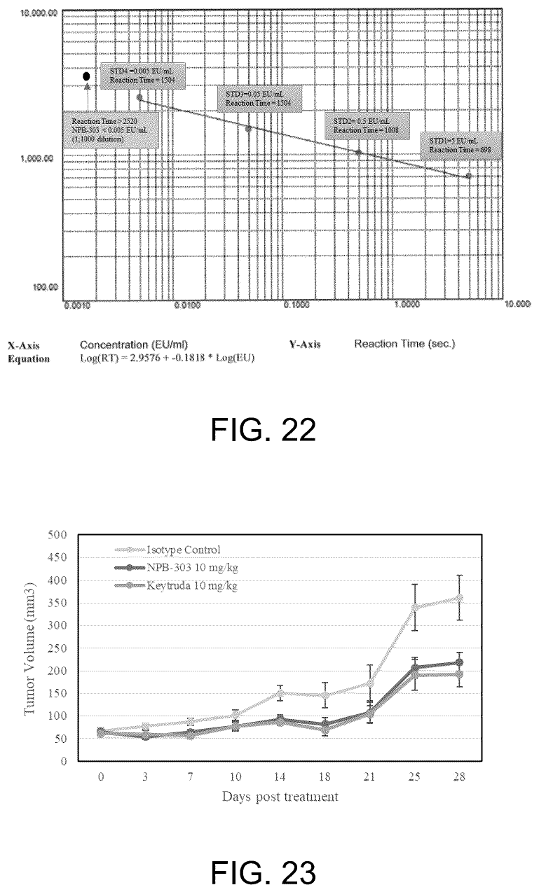

FIG. 22 provides the endotoxin testing performed using a Kinetic Turbidimetric and Kinetic Chromogenic Testing method (ENDOSAFE). The endotoxin level of NPB-303 (1:1000 dilution) was less than 0.005 EU/mL with a reaction time of about 2520 s.

### Example 4

### Anti-Tumor Activity of NPB-303 Antibodies

Two different human triple-negative breast cancer (TNBC) cell lines: MDA-MB-231 (ATCC HTB-26) and MDA-MB-468 (ATCC HTB-132) were purchased from the American Type Culture Collection (ATCC). Cells were cultured in Leibovitz's L-15 medium (Thermo Fisher Scientific, Waltham, MA, USA), supplemented with 10% heat-inactivated fetal bovine serum (GE Healthcare Life Sciences, HyClone Laboratories) and 1% penicillin-streptomycin (Thermo Fisher Scientific) at 37° C. with 5% CO₂.

At least 12-week post-human HSC transplantation, 5×10⁶ TNBC MDA-MB-231 or MDA-MB-468 cells, suspended in serum-free medium mixed with Matrigel (Corning Life Sciences), were injected into thoracic mammary fat pads in female humanized mice. The tumor volumes were monitored weekly using a digital caliper.

Once tumor sizes reached to ˜100 mm³, ˜300 mm³ or ˜600 mm³ in tumor (MDA-MB-231)-bearing humanized mice, mice were sacrificed, and tumor tissues were analyzed for human pDC infiltration using the Flow Cytometry pDC panel as described previously. In a separated experiment, once tumor sizes reached to ˜300 mm³, blood, spleen, tumor, lung and liver were isolated and analyzed for human pDC and T cell infiltration. For T cell Flow Cytometry analysis, cells were stained with Live/Dead Dye, mouse CD45.1 APCCy7 and human antibodies including CD3 PECy7, CD4 PE, CD8 BV515, PD1 BV421 and Tim3 APC (all from BioLegend). Cells were analyzed on a BD FACSCelesta Instrument and data was analyzed using FlowJo software.

Once tumors reached ˜80-100 mm³, TNBC (MDA-MB-231)-bearing humanized NSG mice were randomized into 4 treatment groups. Mice were treated with NPB-303 at doses of 3, 10, 30 mg/kg IV or isotype control antibody (30 mg/kg) twice per week for 4 weeks. Tumor volume, body weight and crude observations were recorded twice weekly. At the end of the study, spleen and tumor were analyzed for pDC infiltration as described before.

For the second TNBC (MDA-MB-468) model development, HIT-TNBC mice were randomized and treated with NPB-303 at doses of 1 and 10 mg/kg and isotype control antibody (10 mg/kg) twice a week, i.v, for three weeks. Tumor volume, body weight and crude observations were recorded twice weekly.

For the combination study, TNBC (MDA-MB-231) bearing humanized NBSGW (NOD.Cg-Kit^{W-41J} Tyr⁺Prkdc^{scid} Il2rg^{tmlWjl}/ThomJ) mice were used. Once tumor volumes reached ˜200 mm3, mice were randomized into four treatment groups: isotype control 30 mg/kg, IV, twice a week for 3 weeks; ABRAXANE® alone, 5 mg/kg, i.p, daily for 5 days only, NPB-303, 30 mg/kg i.v. twice a week for 3 weeks; and the combination of ABRAXANE® (5 mg/kg) and NPB-303 (30 mg/kg) with the same treatment frequencies as mono treatment group. Tumor volumes were recorded twice weekly. Mice were scarified at the end of the study, cells from spleen, tumor and blood were analyzed. Liver tissues also were isolated for histology analysis.

Liver tissues were removed from the Combination Study mice and immediately placed into 4% paraformaldehyde for fixation for up to 48 hours. Then tissues were moved into 70% ethanol until embedding. Each tissue was step-sectioned every 100 μm [microns]), typically resulting 10 slides per organ, and stained with H&E.

Samples were evaluated by a board-certified veterinary pathologist (HORUS Scientific & NOVA Preclinical Consultants, LLC) for morphologic evidence and magnitude of tumor infiltrates into the lung and liver tissues. Light microscopy was used to characterize the tumor infiltrates using the following semi-quantitative scale: (0) no observable change; (1) minimal—a nearly imperceptible/inconspicuous feature/change in the tissue; (2) mild/subtle feature/presence in the tissue; (3) moderate/notable feature/presence in the tissue; (4) marked/overwhelming feature/presence in the tissue. The metastasis scores of all samples were analyzed in Microsoft EXCEL™ and the p-values between groups was calculated using a t-test.

Given the functional human immunity developed in the NSG/NRG-hu HSC mice, these mice partially reject human tumors that are co-engrafted subcutaneously. NSG-hu HSC mice support non-HLA matched tumor growth, and human immune cells can be enhanced to control tumor growth. Described herein are such humanized mice with human immune cells and tumors (“HIT mice”).

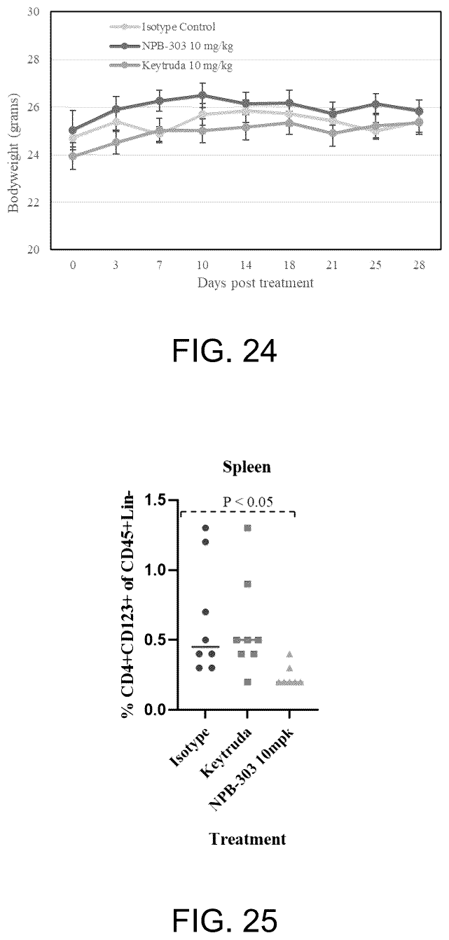

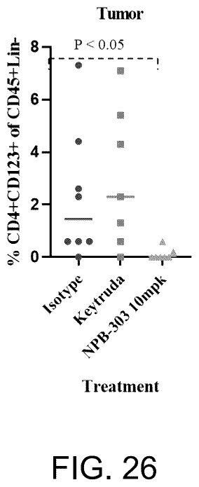

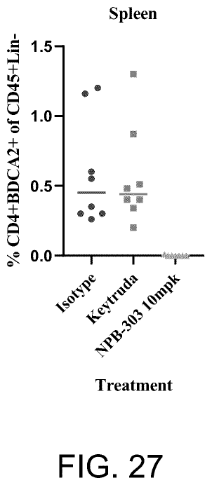

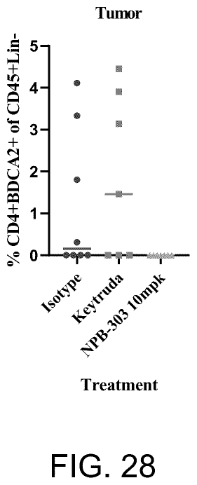

Further, immuno-oncology checkpoint inhibitor anti-PD1 mAb KEYTRUDA® can reactivate anti-tumor responses to control tumor growth. NPB-303 shows comparable tumor volume reduction compared to KEYTRUDA® in tumor-bearing humice (FIG. 23), and the tumor-bearing humice appeared to tolerate the NPB-303 treatment well (FIG. 24). However, spleen and tumor pDC are depleted by NPB-303 but not KEYTRUDA® (FIG. 25 and FIG. 26). Similarly spleen and tumor BDCA-2 was down-regulated by NPB-303 but not KEYTRUDA® (FIG. 27 and FIG. 28).

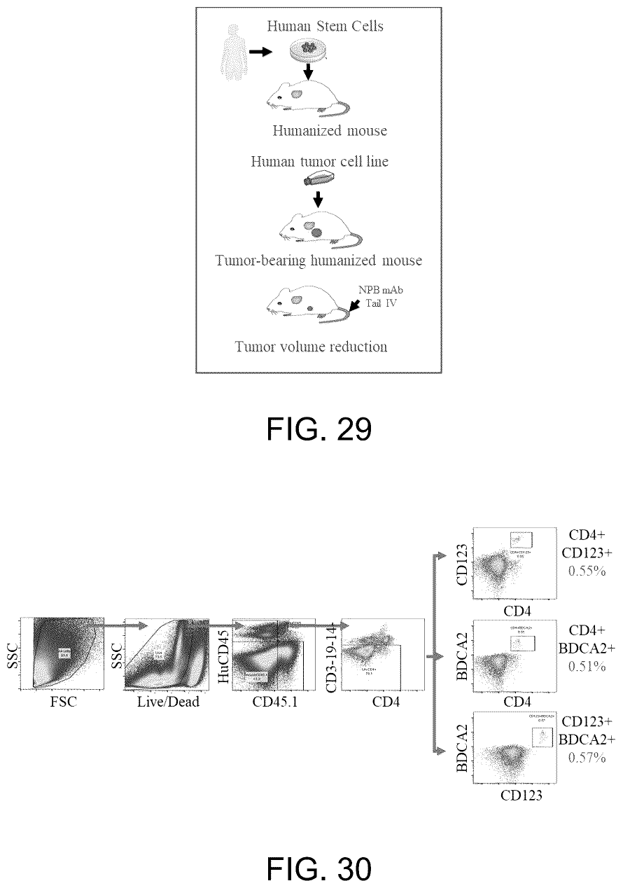

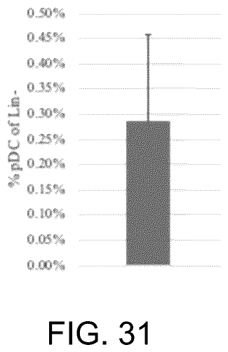

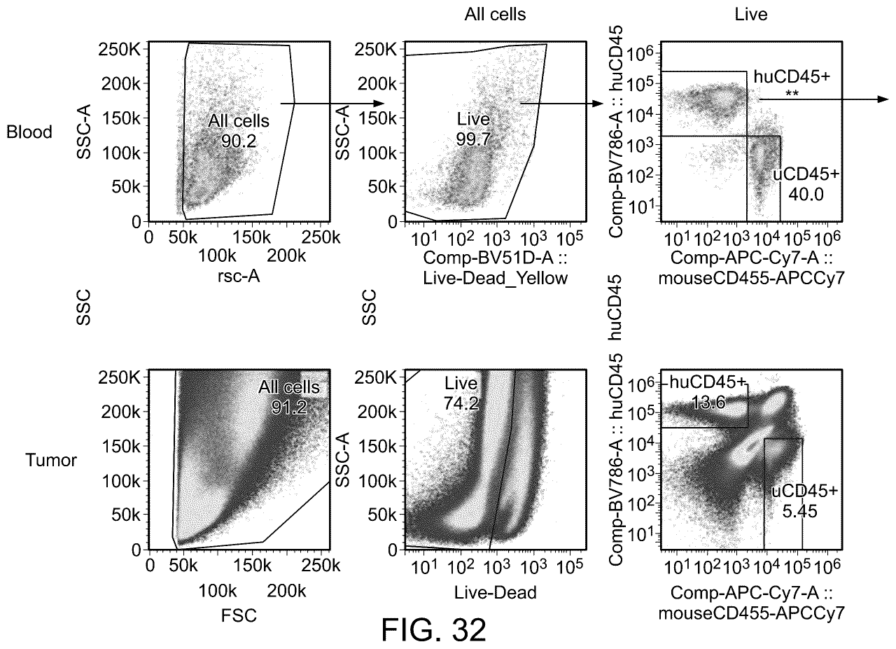

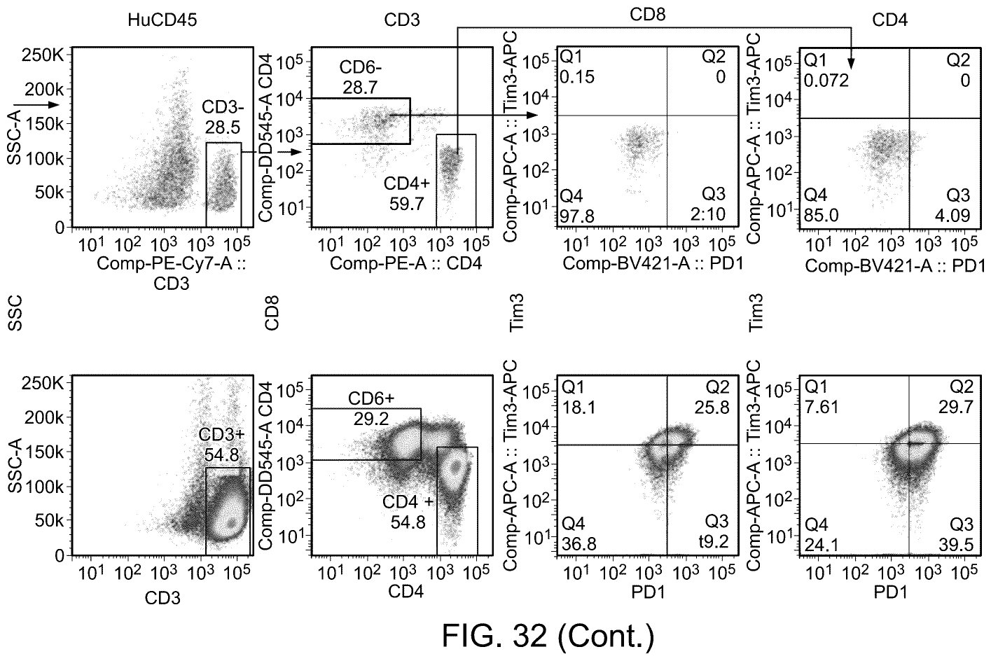

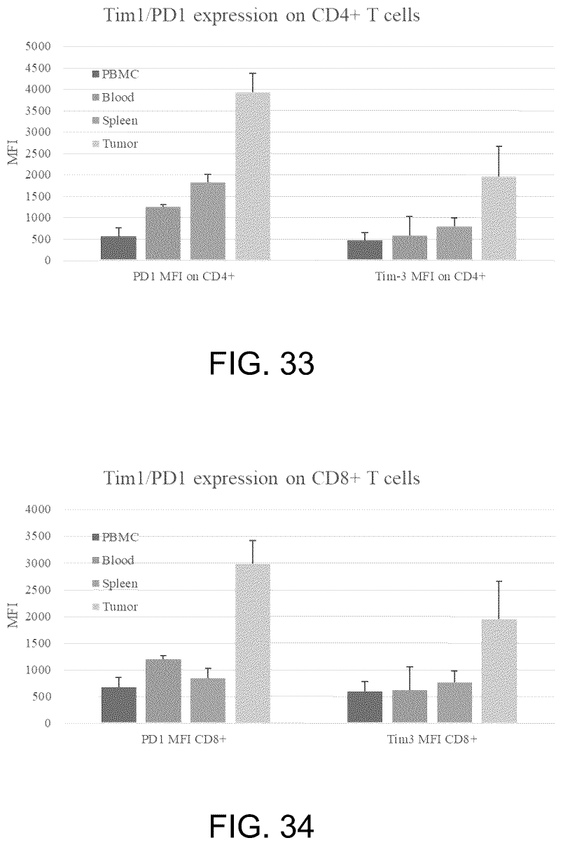

Using the HIT mice with human triple negative breast cancer cells (“TNBC-HIT”), as shown in FIG. 29, it was shown that engrafted tumors were infiltrated with human CD45 leukocytes including CD123+CD4+CD303+ pDCs (FIG. 30 and FIG. 31). In addition, human T cells were detected in the tumor that exhibited immune exhaustion markers (PD1 and TIM3, FIG. 32, FIG. 339, and FIG. 34).

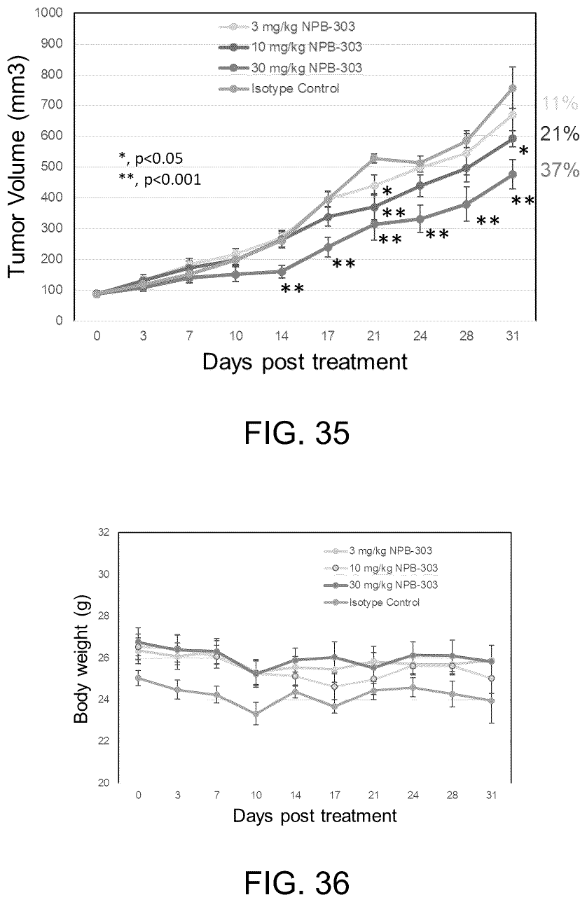

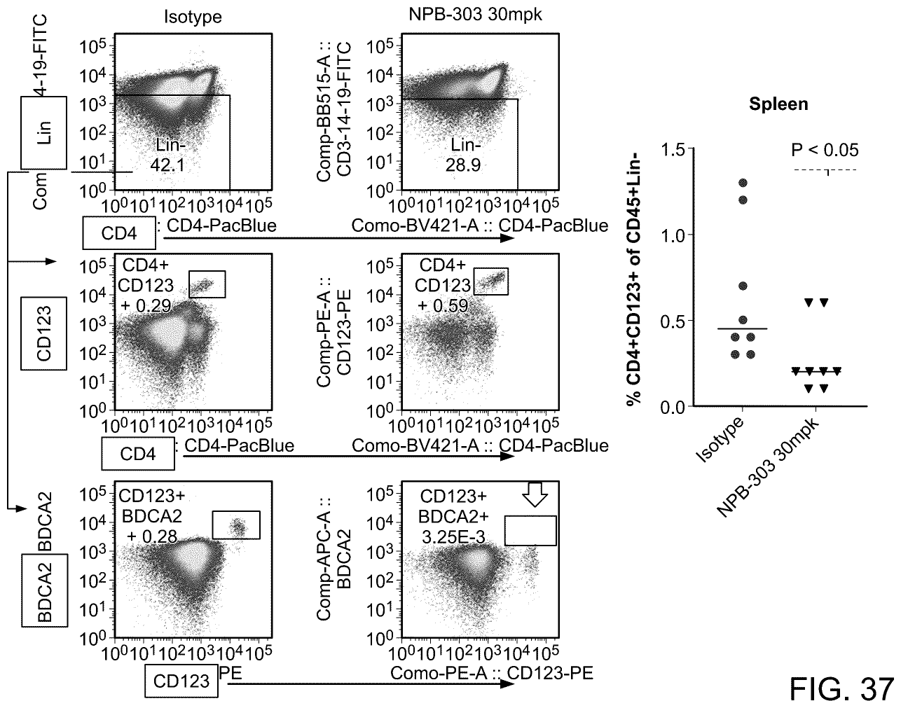

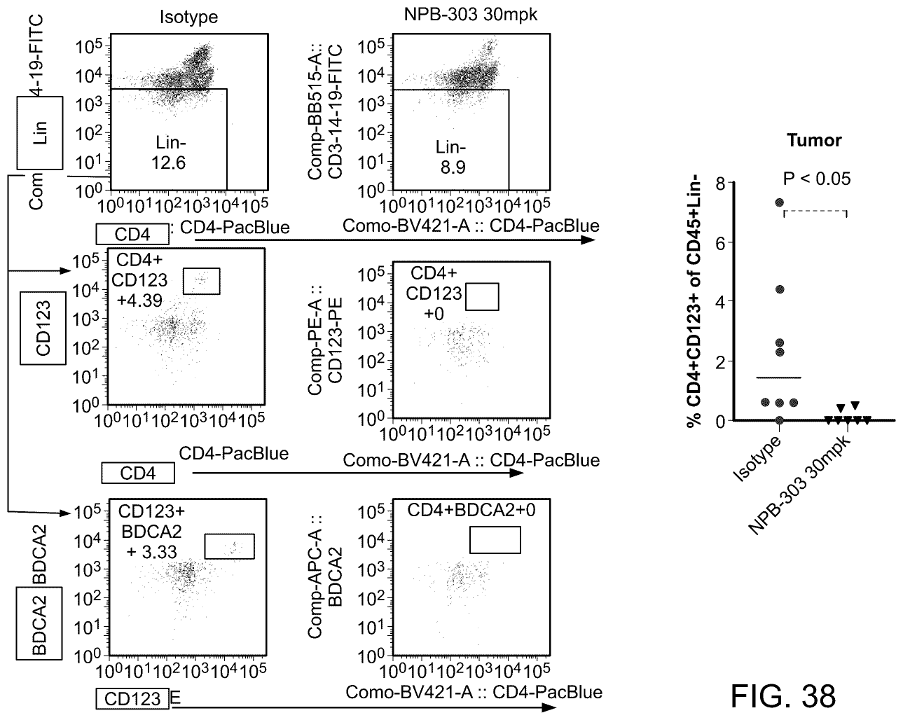

NPB-303 was examined for treating human TNBC in the TNBC-HIT model (FIG. 35). HIT mice with MDA-MB-231 TNBC at about 100 mm³ were treated with NPB-303 at doses of 3 mg/kg, 10 mg/kg and 30 mg/kg, or isotype control mAb (30 mg/kg), and tumor growth was monitored for 31 days. All NPB-303 treated groups had significantly smaller tumor volumes compared to the isotype control mAb treated group, and a dose-dependent response was observed (FIG. 35). No apparent adverse effect was observed by the mAb (FIG. 36). As expected, human pDC was significantly depleted in both tumors (FIG. 37) and spleens (FIG. 38) of NPB-303-treated mice. The remaining pDC also lost expression of BDCA2/CD303, indicating NPB-303 binding to and inhibiting all human pDC.

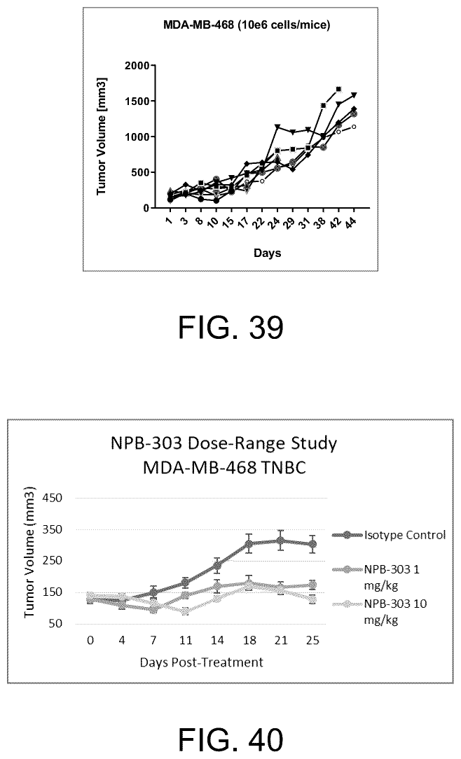

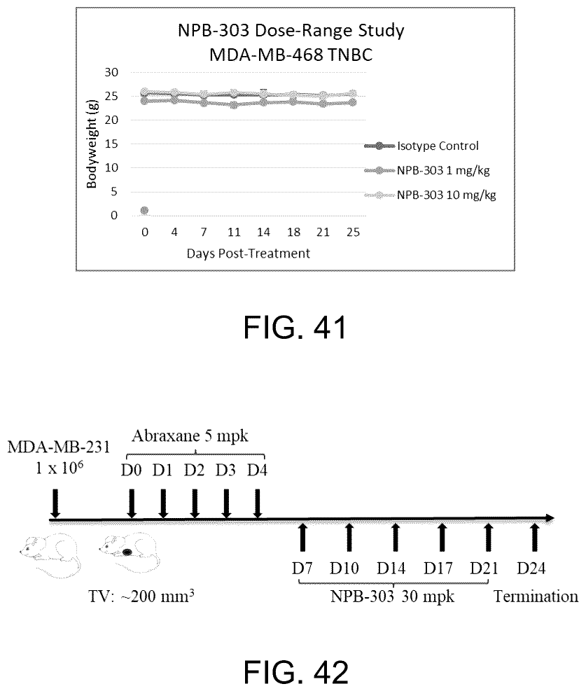

To confirm the therapeutic activity of NPB-303 on human TNBC, its effect in the TNBC-HIT mice engrafted with another human TNBC cell line (MDA-MB-468, FIG. 39) was tested. Similar tumor reduction activity was observed in the MB-468-HIT mice (FIG. 40), with no obvious adverse effect on HIT mice (FIG. 41).

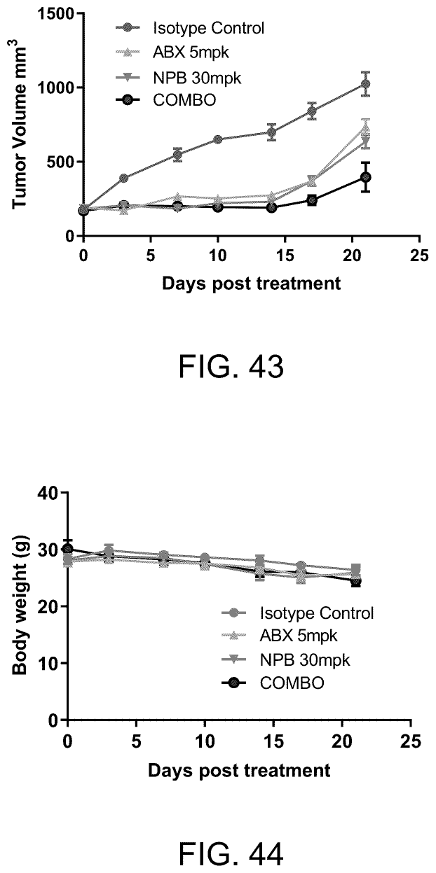

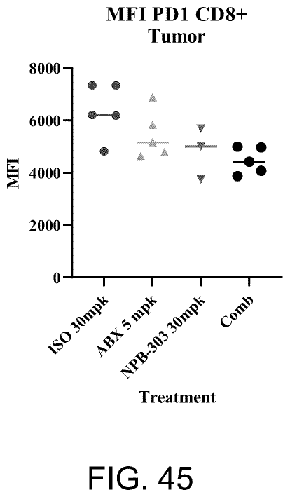

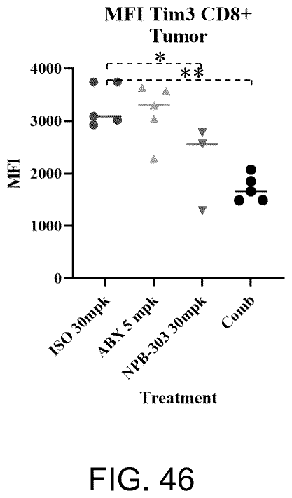

Patients with TNBC are currently treated with the frontline chemotherapy of ABRAXANE®, followed with immune check point inhibitors such as anti-PD1 mAb (KEYTRUDA®). The combination activity of NPB-303 following ABRAXANE® therapy was thus tested in TNBC-HIT mice (FIG. 42). When tumor sizes were followed after treatments, ABRAXANE® or NPB-303 each inhibited tumor growth to similar size (˜50%, FIG. 43). Importantly, combination of Abx/NPB-303 further reduced the tumor size by ˜90% (FIG. 43) without obvious adverse effect on HIT mice (FIG. 44). Compared to the group treated with either ISO or ABRAXANE® alone, treatments with NPB-303 or in combination with ABRAXANE® significantly reversed tumor infiltrating CD8 T cells by down-regulation of PD1 and Tim3 expression (FIG. 45 and FIG. 46).

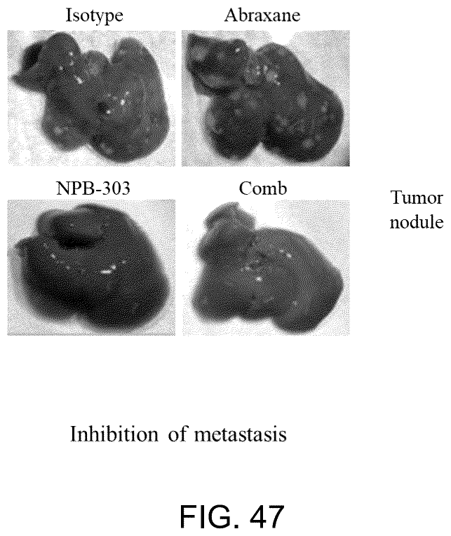

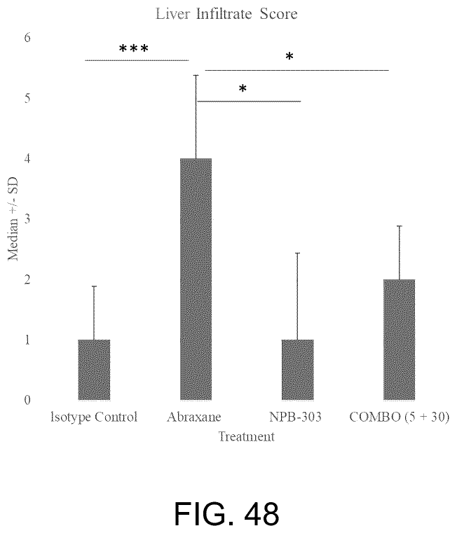

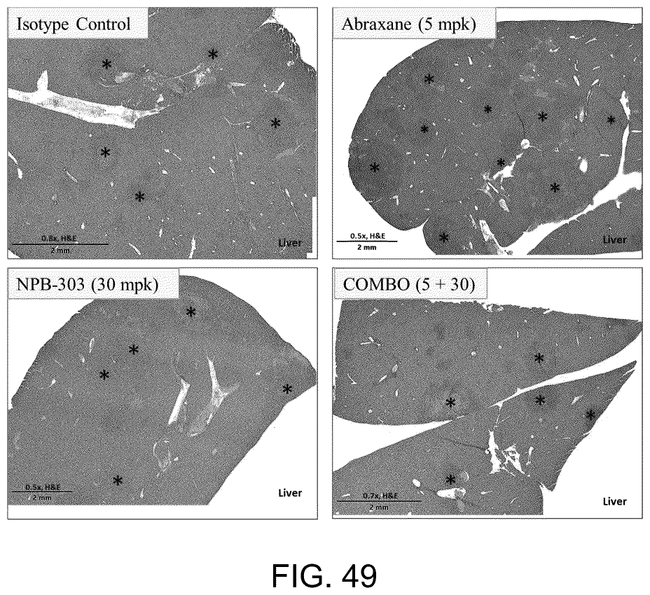

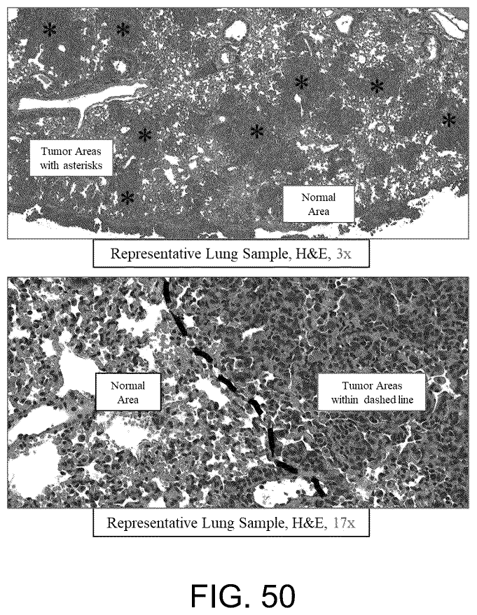

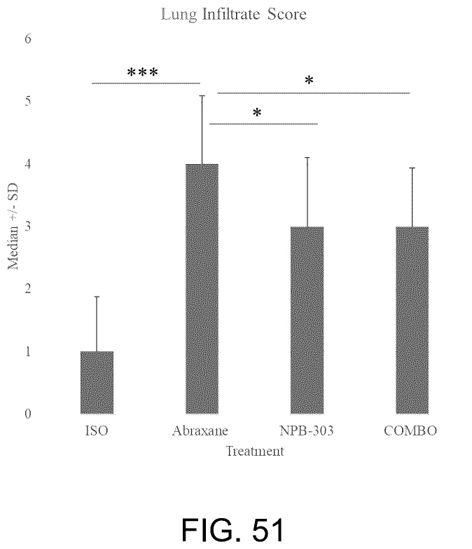

ABRAXANE® treatments may lead to tumor metastasis. Metastasized tumors were, thus, investigated in TNBC-HIT mice (FIG. 43). While ABRAXANE®-treated mice showed clear tumor metastasis to the liver, NPB-303-treated mice showed no or lower tumor metastasis. A combination of ABRAXANE® and NPB-303 showed significantly reduced tumor metastasis in liver (FIG. 47, FIG. 48, and FIG. 49) and in lung (FIG. 50 and FIG. 51). These data indicate that NPB303-induced tumor inhibition was not associated with enhanced tumor metastasis, and NPB-303 combination with ABRAXANE® cooperatively suppressed tumor growth and reduced tumor metastasis.

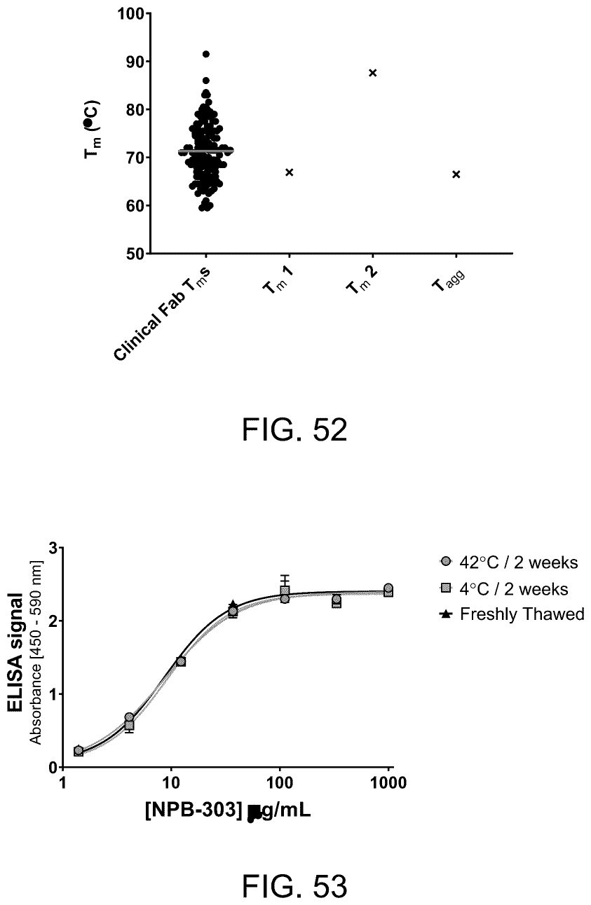

NPB-303 also displays a thermal stability profile that is similar to that of other clinical stage and marketed monoclonal antibodies. As shown in FIG. 52, the thermal denaturation (Tₘ) and aggregation onset (T_{agg}) for NPB-303 is comparable to that of therapeutic antibodies that progressed at least to clinical trials. Further, NPB-303 binding activity is stable under various heat stress conditions. FIG. 53 shows the binding activities of NPB-303 at different conditions. There are no differences between fresh thawed NPB-303, NPB-303 incubated at 4° C. for 2 weeks, and NPB-303 subjected to stress incubation at 42° C. for 2 weeks.

pDC/IFN-I in tumor microenvironments is associated with immune suppression. NPB-303 inhibited TA-pDC in a dose-dependent response in TNBC-HIT mice, which was confirmed with TNBC-HIT mice with two human TNBC cell lines (MB231 and MB468). This effect of NPB-303 was examined in combination with the first line chemotherapy agent, ABRAXANE®. Either ABRAXANE® or NPB-303 reduced tumor growth, and their combination synergistically inhibited tumors. NPB-303 showed significant activity in preventing tumor metastasis to the liver and lung. Even though anti-PD1 antibodies, such as KEYTRUDA®, have shown some efficacy in treating various types of human cancers, the majority of cancer patients fail to respond to KEYTRUDA® due to other immune suppressive mechanisms including persistent inflammation. This Example provides a new weapon for effective cancer treatment.

### Example 5

### NPB-303 Antibody Developability and Manufacturability Engineering

With the entry into clinical trials and the development of candidate drugs increasing, the testing for the safety and efficacy evaluation has become more important. Compared with other molecular drugs, monoclonal antibodies are more susceptible to various post-translational modifications (PTM) during production, transportation, and storage, which will affect the stability of the drug molecule and the structure and function of the protein. Aspartate isomerization is a spontaneous non-enzymatic PTM method. After isomerization of aspartic acid, a methylene group is inserted into the protein backbone, that is, the aspartate side chain reduced a methylene group, thus causing a change in the structure of the protein and protein function. Many studies have revealed that aspartate isomerization can affect the effectiveness of candidate drugs. For example, aspartic acid isomerization in the complementarity-determining regions (CDRs) of antibodies can reduce the receptor binding efficiency and can affect final drug efficacy.

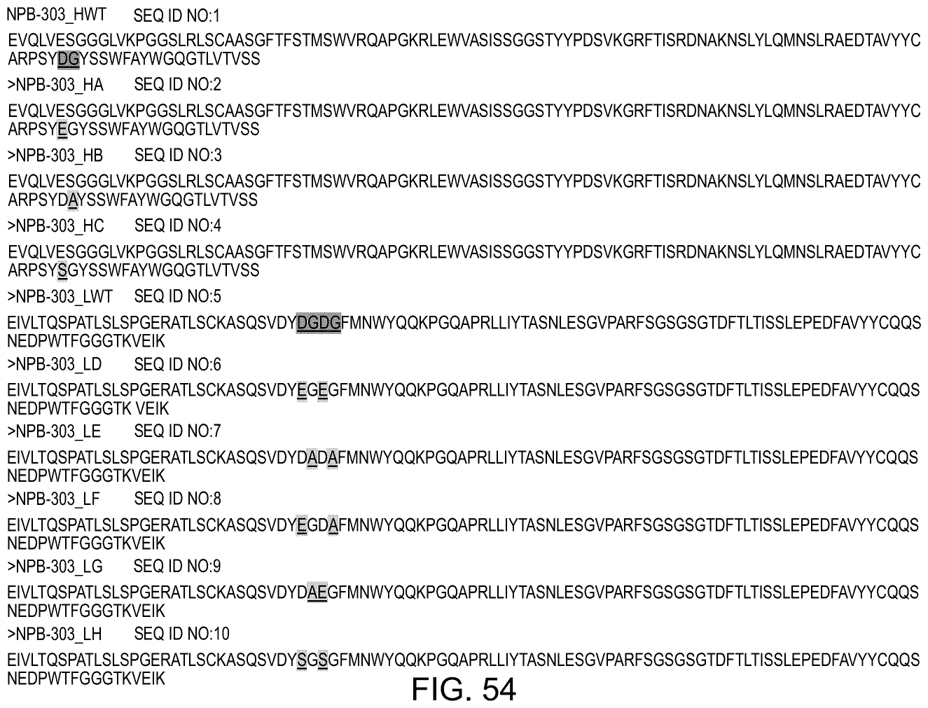

In the NPB-303 sequence, DG could be problematic amino acids which causes isomerization issue. A developability engineering study was conducted by following modification of the antibody sequences: 1) change DG in heavy chain CDR3 to DA, EG and SG without change light chain to determine if the changes affect affinity; 2), change DGDG in light chain CDR1 to EGEG, DADA, EGDA, DAEG, SGSG (maybe SGDG and DGSG) without change heavy chain to determine if the changes affect affinity; and 3), combine the best changes in heavy and light chains into the same Ab molecule. The detailed sequence modification is shown in the FIG. 54, which includes SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

The antibodies with different combinations of heavy chain and light chain combinations (Table 3) were produced in a small scale (3 mL) using transient antibody production. After protein purification, the antibodies were analyzed including titer, A280, SEC-HPLC and Caliper SDS (reducing and non-reducing). The summary of QC data for all antibodies is shown in Table 4. All antibodies were examined in a formulation comprising 50 mM Na-AC, 33 mM Tris, and the indicated amount of antibody, with a pH of 5.0.

TABLE 3

| Antibody ID | Heavy chain construct | Light chain construct |
|---|---|---|
| NPB-303 WT | SEQ ID NO: 1 | SEQ ID NO: 5 |
| NPB-303_A | SEQ ID NO: 2 | SEQ ID NO: 5 |
| NPB-303_B | SEQ ID NO: 3 | SEQ ID NO: 5 |
| NPB-303_C | SEQ ID NO: 4 | SEQ ID NO: 5 |
| NPB-303_D | SEQ ID NO: 1 | SEQ ID NO: 6 |
| NPB-303_E | SEQ ID NO: 1 | SEQ ID NO: 7 |
| NPB-303_F | SEQ ID NO: 1 | SEQ ID NO: 8 |
| NPB-303_G | SEQ ID NO: 1 | SEQ ID NO: 9 |
| NPB-303_H | SEQ ID NO: 1 | SEQ ID NO: 10 |
| NPB-303_A.D | SEQ ID NO: 2 | SEQ ID NO: 6 |
| NPB-303_A.E | SEQ ID NO: 2 | SEQ ID NO: 7 |
| NPB-303_A.F | SEQ ID NO: 2 | SEQ ID NO: 8 |
| NPB-303_A.G | SEQ ID NO: 2 | SEQ ID NO: 9 |
| NPB-303_A.H | SEQ ID NO: 2 | SEQ ID NO: 10 |
| NPB-303_B.D | SEQ ID NO: 3 | SEQ ID NO: 6 |
| NPB-303_B.E | SEQ ID NO: 3 | SEQ ID NO: 7 |
| NPB-303_B.F | SEQ ID NO: 3 | SEQ ID NO: 8 |
| NPB-303_B.G | SEQ ID NO: 3 | SEQ ID NO: 9 |
| NPB-303_B.H | SEQ ID NO: 3 | SEQ ID NO: 10 |
| NPB-303_C.D | SEQ ID NO: 4 | SEQ ID NO: 6 |
| NPB-303_C.E | SEQ ID NO: 4 | SEQ ID NO: 7 |
| NPB-303_C.F | SEQ ID NO: 4 | SEQ ID NO: 8 |
| NPB-303_C.G | SEQ ID NO: 4 | SEQ ID NO: 9 |
| NPB-303_C.H | SEQ ID NO: 4 | SEQ ID NO: 10 |

TABLE 4

| | | | | Reduced |
|---|---|---|---|---|
| | Concentration | Amount | SEC-HPLC | CD-SDS |
| Antibody ID | (mg/ml) | (mg) | Purity (%) | Purity (%) |
| NPB-303 WT | 0.21 | 0.2 | 98.7 | 98.72 |
| NPB-303_A | 0.44 | 0.2 | 97.7 | 98.43 |
| NPB-303_B | 0.52 | 0.2 | 96.7 | 98.47 |
| NPB-303_C | 0.45 | 0.2 | 97.0 | 98.90 |
| NPB-303_D | 0.49 | 0.2 | 97.8 | 98.53 |
| NPB-303_E | 0.16 | 0.1 | 99.3 | 99.19 |
| NPB-303_F | 0.55 | 0.2 | 97.6 | 98.07 |
| NPB-303_G | 0.37 | 0.2 | 98.9 | 98.79 |
| NPB-303_H | 0.46 | 0.2 | 96.9 | 98.83 |
| NPB-303_A.D | 0.50 | 0.2 | 96.7 | 98.83 |
| NPB-303_A.E | 0.31 | 0.2 | 98.5 | 98.47 |
| NPB-303_A.F | 0.21 | 0.2 | 98.4 | 98.68 |
| NPB-303_A.G | 0.21 | 0.1 | 99.4 | 98.66 |
| NPB-303_A.H | 0.47 | 0.2 | 97.1 | 98.78 |
| NPB-303_B.D | 0.66 | 0.2 | 96.7 | 98.72 |
| NPB-303_B.E | 0.44 | 0.2 | 97.2 | 98.59 |
| NPB-303_B.F | 0.58 | 0.2 | 96.1 | 98.76 |
| NPB-303_B.G | 0.25 | 0.1 | 97.5 | 98.59 |
| NPB-303_B.H | 0.27 | 0.2 | 96.9 | 98.93 |
| NPB-303_C.D | 0.61 | 0.2 | 95.3 | 98.92 |
| NPB-303_C.E | 0.32 | 0.2 | 98.1 | 98.94 |
| NPB-303_C.F | 0.51 | 0.2 | 96.4 | 99.02 |
| NPB-303_C.G | 0.37 | 0.2 | 98.2 | 98.85 |
| NPB-303_C.H | 0.17 | 0.2 | 97.7 | 98.90 |

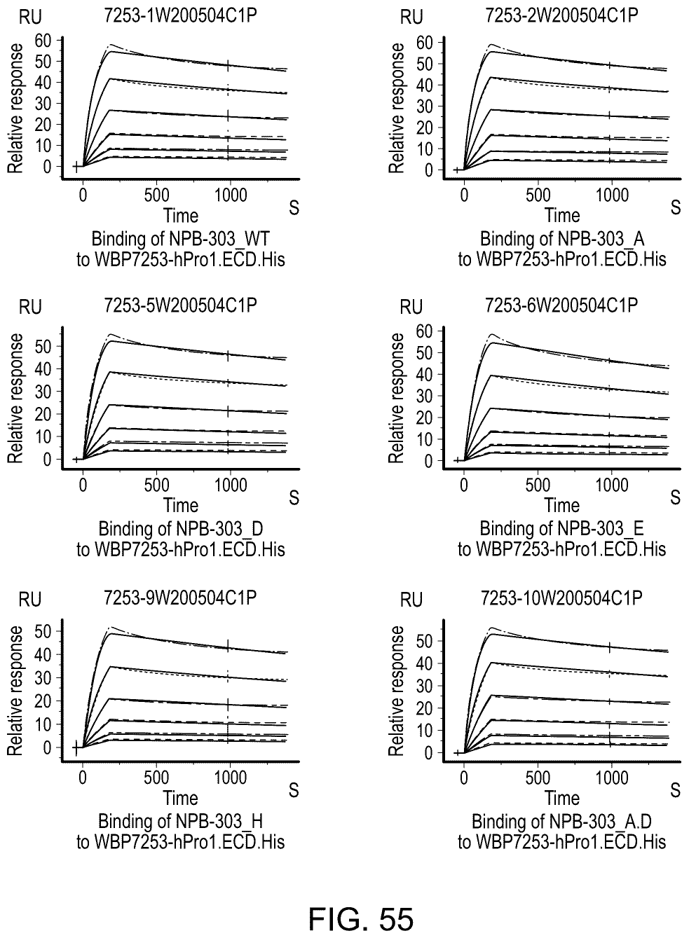

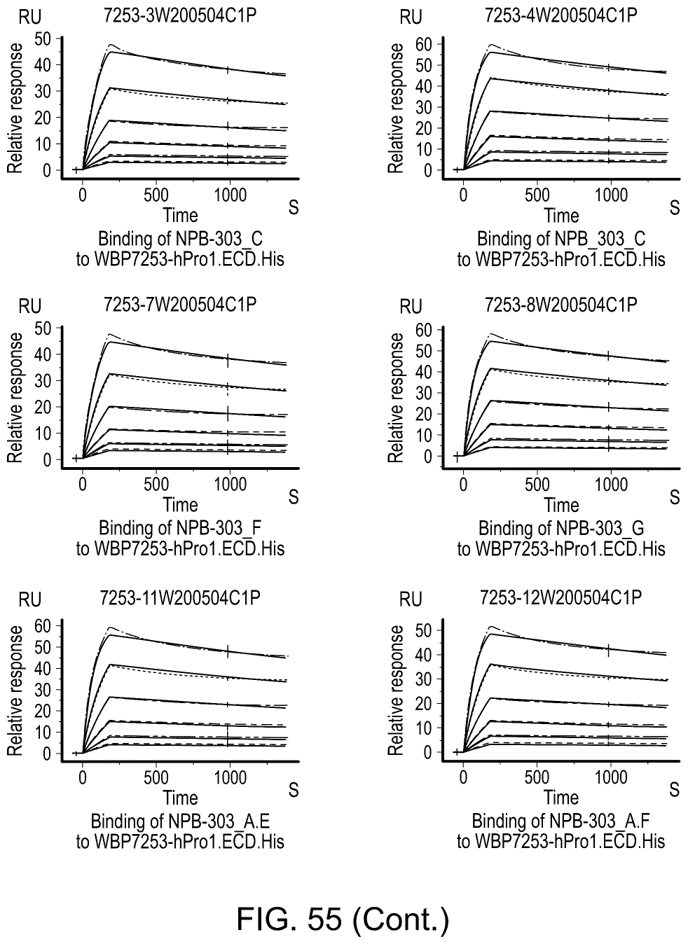

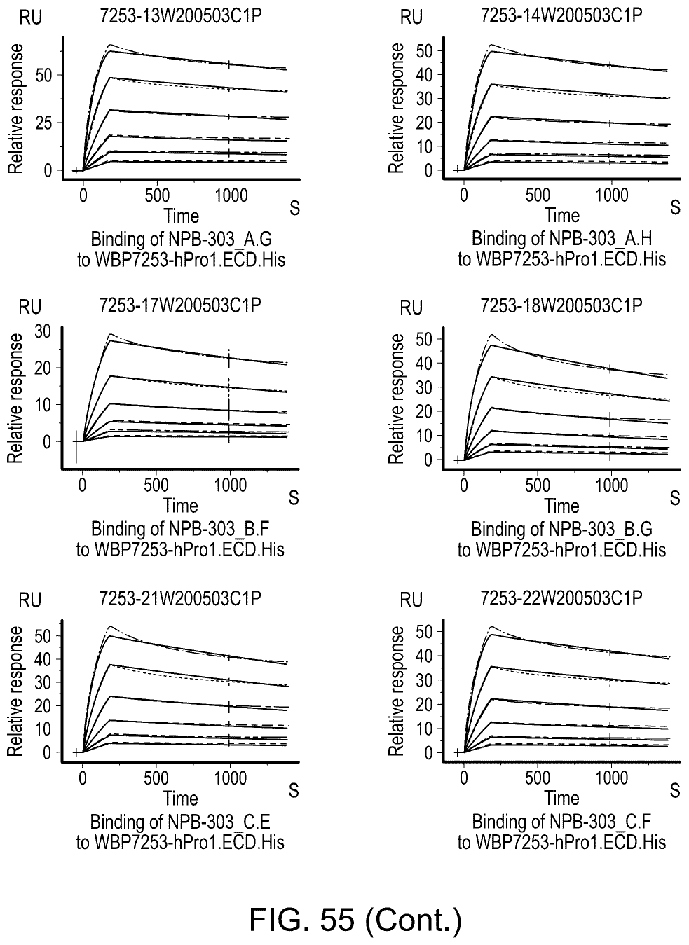

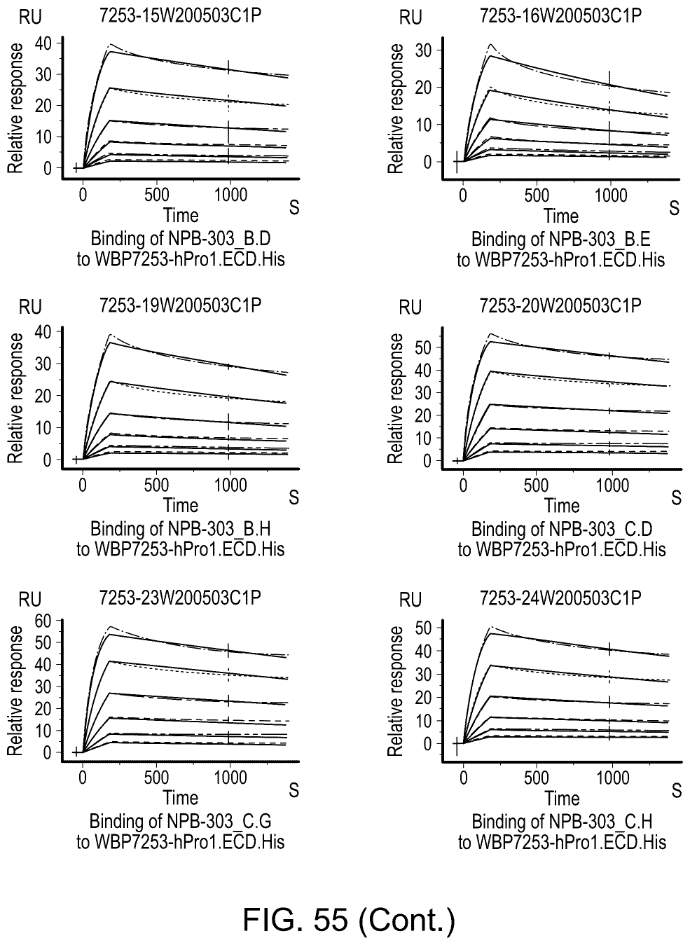

BDCA2 antibody binding kinetics were measured using a Biacore surface plasmon resonance (SPR) instrument (GE Life Sciences). Briefly, anti-BDCA2 antibodies were captured on an anti-human Fc chip. The recombinant BDCA2 proteins were then flowed over the captured antibodies over a range of concentrations, at a rate of 50 μL/minute. The data was analyzed to determine kinetic constants using the BIAevaluation software package (GE Life Sciences). The sensorgrams of SPR based affinity testing for all 24 antibodies are shown in FIG. 55, and the biophysical characteristics of all 24 antibodies are summarized in Table 5.

TABLE 5

| Antibody ID | kₐ (Ms⁻¹) | k_{d} (s⁻¹) | KD (M) |
|---|---|---|---|
| NPB-303 WT | 1.31 × 10⁵ | 1.56 × 10⁻⁴ | 1.19 × 10⁻⁹ |
| NPB-303_A | 1.44 × 10⁵ | 1.45 × 10⁻⁴ | 1.01 × 10⁻⁹ |
| NPB-303_B | 9.39 × 10⁴ | 1.93 × 10⁻⁴ | 2.06 × 10⁻⁹ |
| NPB-303_C | 1.38 × 10⁵ | 1.65 × 10⁻⁴ | 1.2 × 10⁻⁹ |
| NPB-303_D | 1.15 × 10⁵ | 1.44 × 10⁻⁴ | 1.25 × 10⁻⁹ |
| NPB-303_E | 1.06 × 10⁵ | 2.08 × 10⁻⁴ | 1.96 × 10⁻⁹ |
| NPB-303_F | 1.07 × 10⁵ | 1.87 × 10⁻⁴ | 1.74 × 10⁻⁹ |
| NPB-303_G | 1.26 × 10⁵ | 1.66 × 10⁻⁴ | 1.31 × 10⁻⁹ |
| NPB-303_H | 9.88 × 10⁴ | 1.66 × 10⁻⁴ | 1.68 × 10⁻⁹ |
| NPB-303_A.D | 1.3 × 10⁵ | 1.38 × 10⁻⁴ | 1.06 × 10⁻⁹ |
| NPB-303_A.E | 1.23 × 10⁵ | 1.83 × 10⁻⁴ | 1.49 × 10⁻⁹ |
| NPB-303_A.F | 1.16 × 10⁵ | 1.64 × 10⁻⁴ | 1.41 × 10⁻⁹ |
| NPB-303_A.G | 1.42 × 10⁵ | 1.42 × 10⁻⁴ | 9.98 × 10⁻¹⁰ |
| NPB-303_A.H | 1.07 × 10⁵ | 1.59 × 10⁻⁴ | 1.49 × 10⁻⁹ |
| NPB-303_B.D | 8.68 × 10⁴ | 2.12 × 10⁻⁴ | 2.44 × 10⁻⁹ |
| NPB-303_B.E | 8.56 × 10⁴ | 4.09 × 10⁻⁴ | 4.77 × 10⁻⁹ |
| NPB-303_B.F | 6.76 × 10⁴ | 2.32 × 10⁻⁴ | 3.44 × 10⁻⁹ |
| NPB-303_B.G | 1.09 × 10⁵ | 2.86 × 10⁻⁴ | 2.63 × 10⁻⁹ |
| NPB-303_B.H | 7.85 × 10⁴ | 2.72 × 10⁻⁴ | 3.47 × 10⁻⁹ |
| NPB-303_C.D | 1.22 × 10⁵ | 1.54 × 10⁻⁴ | 1.26 × 10⁻⁹ |
| NPB-303_C.E | 1.25 × 10⁵ | 2.37 × 10⁻⁴ | 1.89 × 10⁻⁹ |
| NPB-303_C.F | 1.10 × 10⁵ | 1.95 × 10⁻⁴ | 1.77 × 10⁻⁹ |
| NPB-303_C.G | 1.38 × 10⁵ | 1.82 × 10⁻⁴ | 1.32 × 10⁻⁹ |
| NPB-303_C.H | 9.74 × 10⁴ | 1.97 × 10⁻⁴ | 2.02 × 10⁻⁹ |

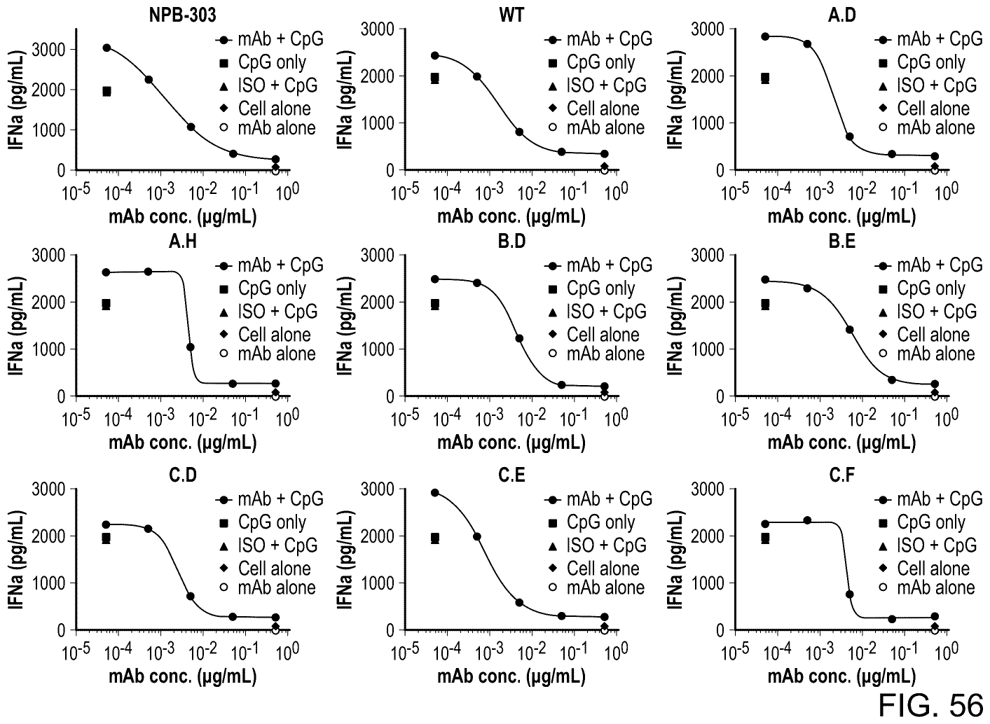

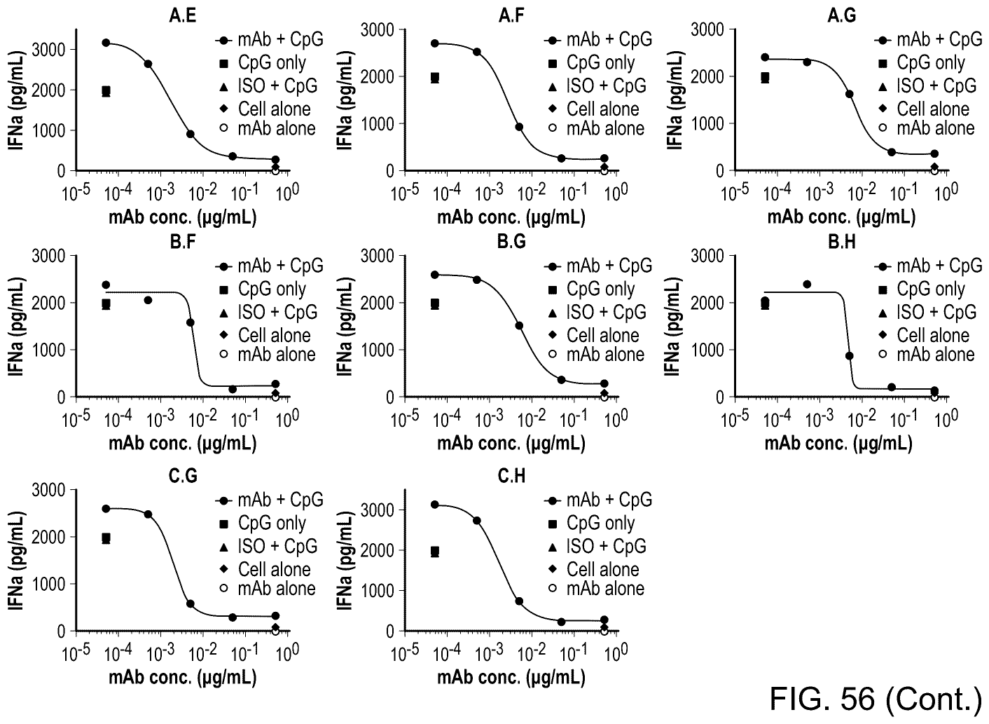

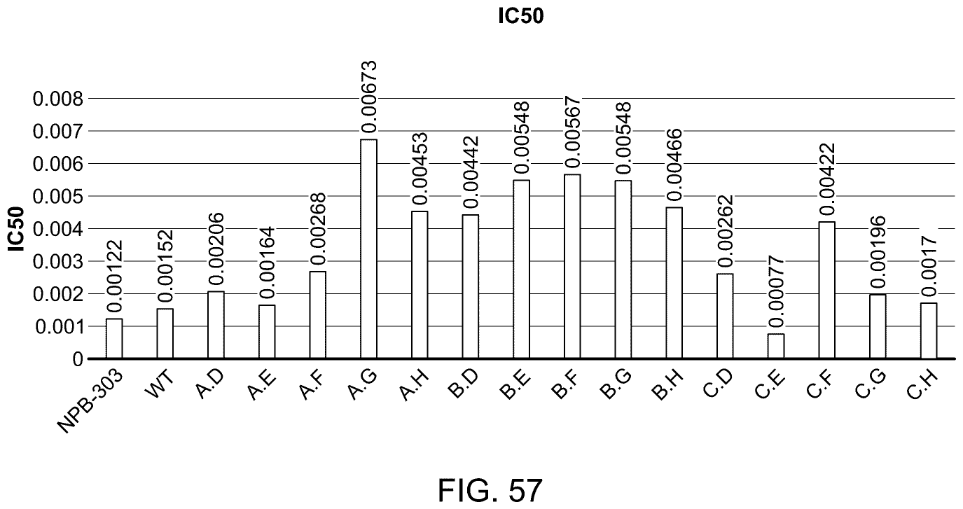

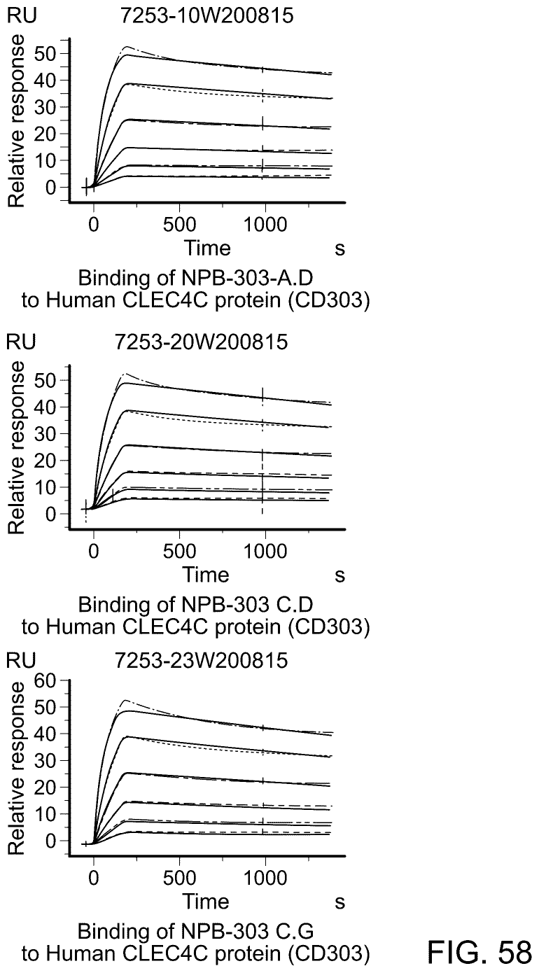

The 24 antibodies were further tested for their biological activity toward inhibition of TLR-9-induced IFNα production in an in vitro cultivated PBMC from one healthy human donor. A set of graphs of IFNα inhibition assay is shown in FIG. 56, and the potencies (IC₅₀) of all antibodies are summarized in FIG. 57. Although the IC₅₀ varies between antibodies, all 24 antibodies show dose-dependent inhibition of TLR-9 induced IFNα production.

Based on the binding affinity, expression level, and potency of all 24 antibodies, the top-3 antibodies were selected (NPB-303_A.D; NPB-303_C.D and NPB-303_C.G), and these antibodies were produced on a larger scale (100 mL) using transient transfection. The summary of QC data for the top-3 antibodies is shown in Table 6. The binding affinities were analyzed using the SPR method, and the sensorgrams of all three are shown in FIG. 58. The biophysical activities are summarized in Table 7. The data indicate that the biophysical characterization of the large-scale production is similar to that of the small-scale production.

TABLE 6

| | Concentration | Amount | SEC-HPLC |
|---|---|---|---|
| Antibody ID | (mg/ml) | (mg) | Purity (%) |
| NPB-303_A.D | 2.66 | 64.74 | 99.5 |
| NPB-303_C.D | 2.67 | 64.80 | 99.3 |
| NPB-303_C.G | 2.24 | 52.48 | 99.6 |

TABLE 7

| Antibody ID | kₐ (Ms⁻¹) | k_{d} (s⁻¹) | KD (M) |
|---|---|---|---|
| NPB-303_A.D | 1.44 × 10⁵ | 1.33 × 10⁻⁴ | 9.27 × 10⁻¹⁰ |
| NPB-303_C.D | 1.42 × 10⁵ | 1.61 × 10⁻⁴ | 1.13 × 10⁻⁹ |
| NPB-303_C.G | 1.58 × 10⁵ | 1.76 × 10⁻⁴ | 1.11 × 10⁻⁹ |

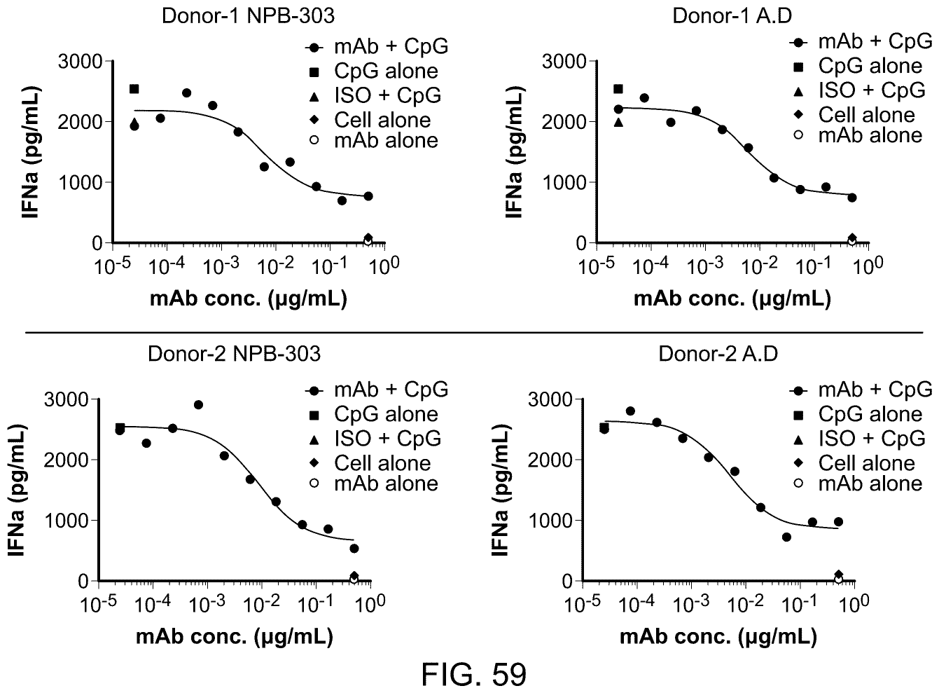

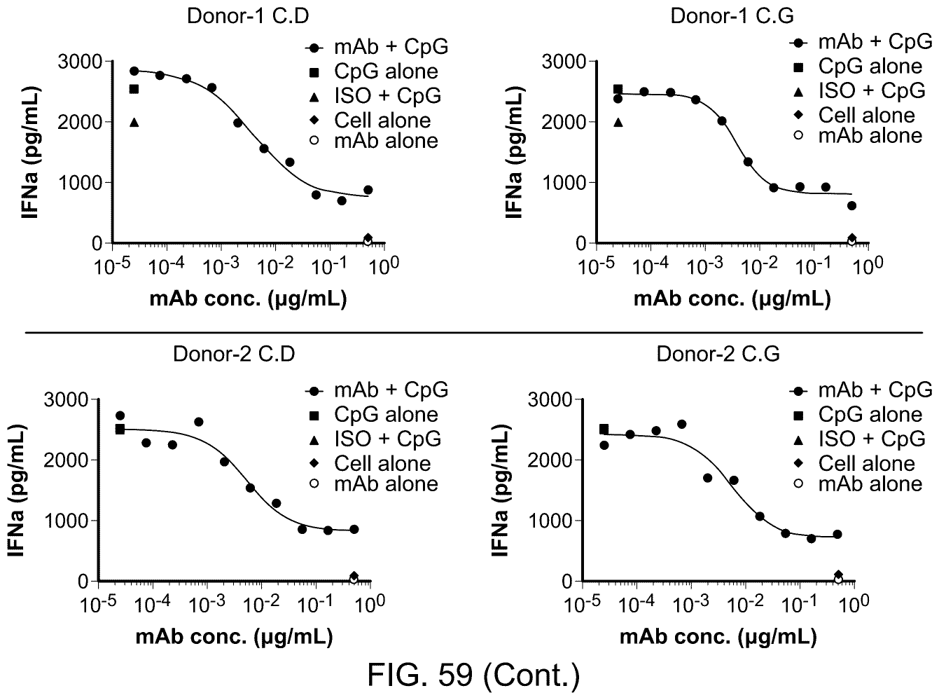

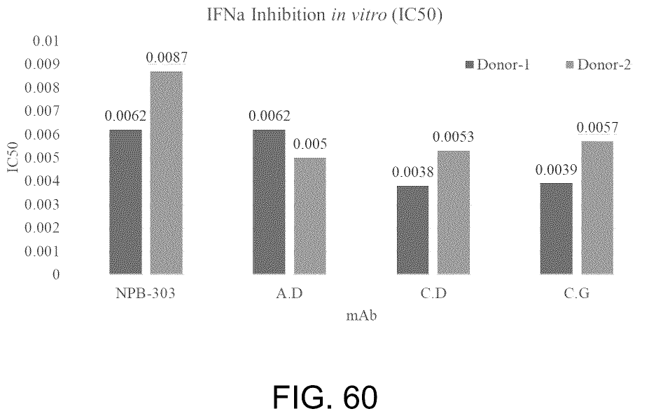

NPB-303_A.D; NPB-303_C.D and NPB-303_C.G from the large-scale production were also tested for inhibition of TLR-9 induced IFNα production. A dose-dependent IFNα inhibition was observed on two different healthy donors (FIG. 59), and the IC₅₀ values shown in FIG. 60 were obtained.

NPB-303_A.D; NPB-303_C.D and NPB-303_C.G from the large-scale production were further tested for human pDC depletion in vivo in the humanized mice. Briefly, 20 humanized mice were randomized into 5 groups (4 mice per group). The mice were treated with either isotype control antibody, NPB-303_WT, NPB-303_A.D, NPB-303_C.D, or NPB-303_C.G at 10 mg/kg i.v. Seventy-two (72) hours later, mice were sacrificed, and the blood and spleen were analyzed for pDCs.

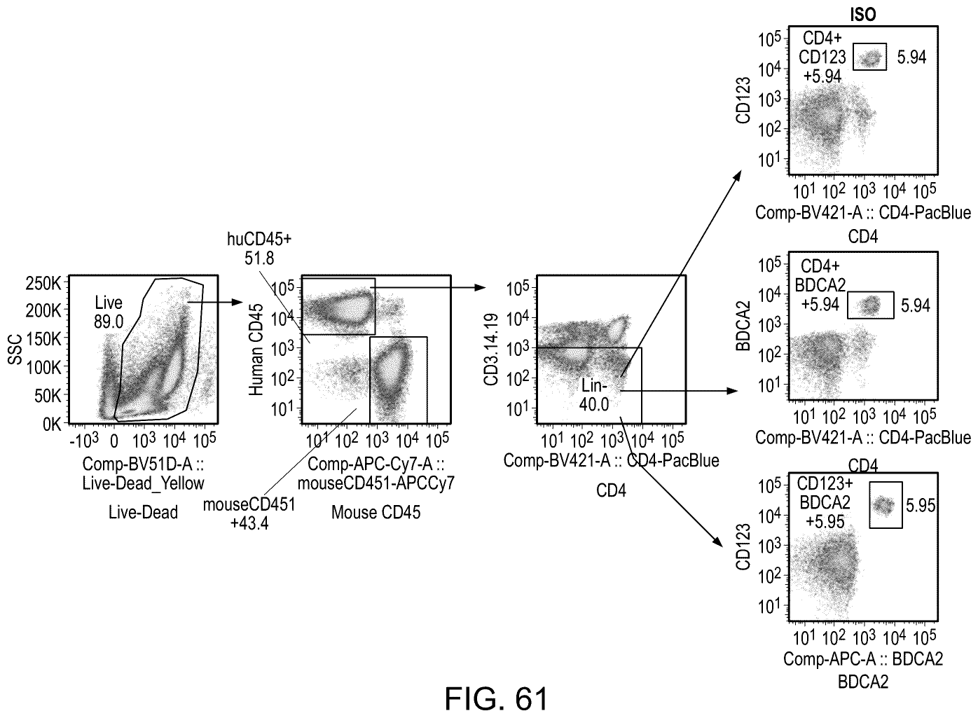

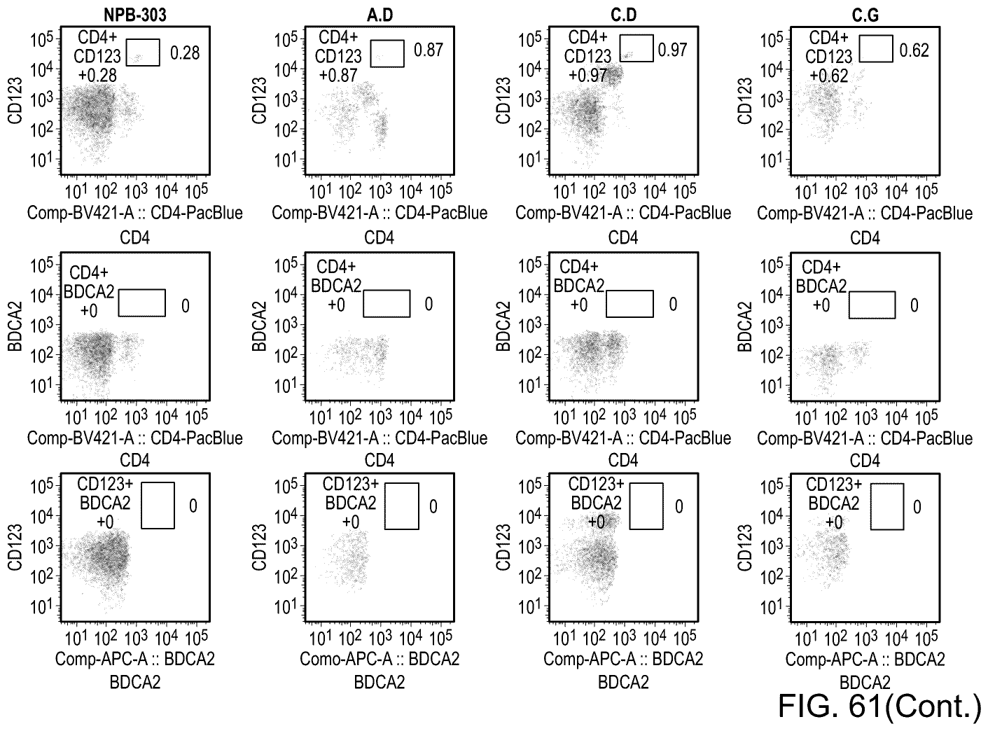

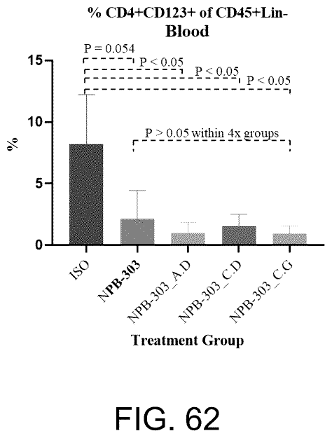

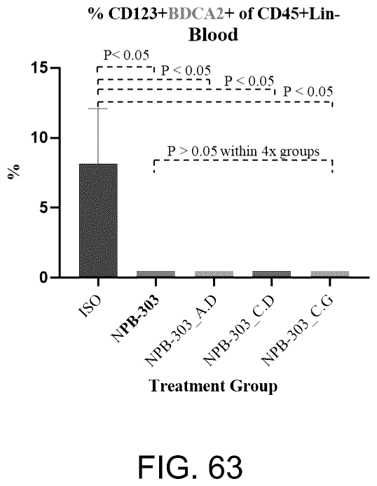

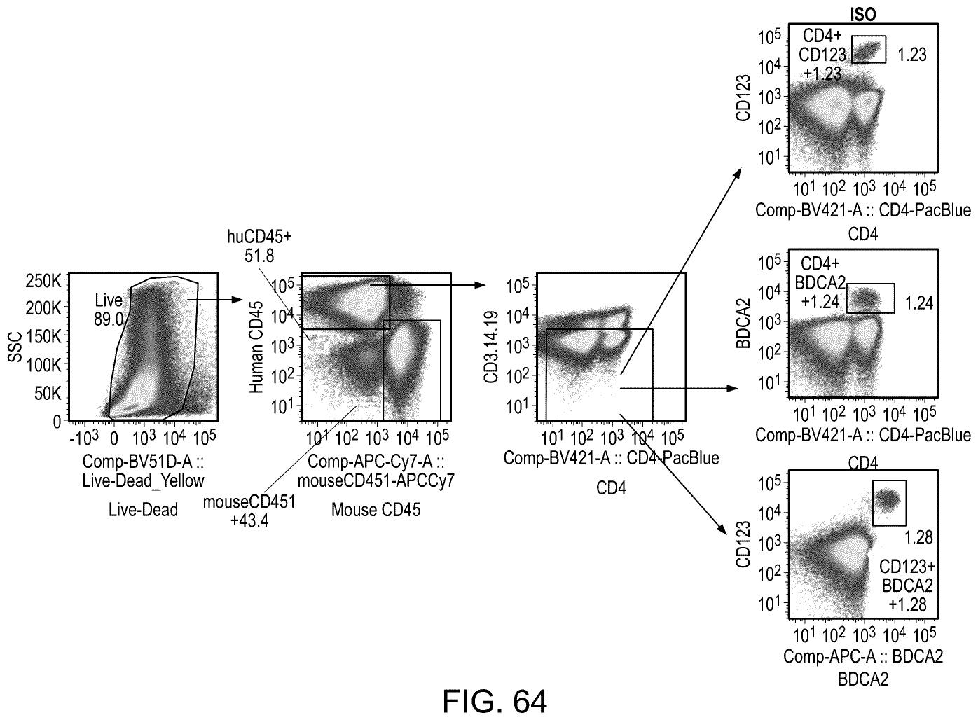

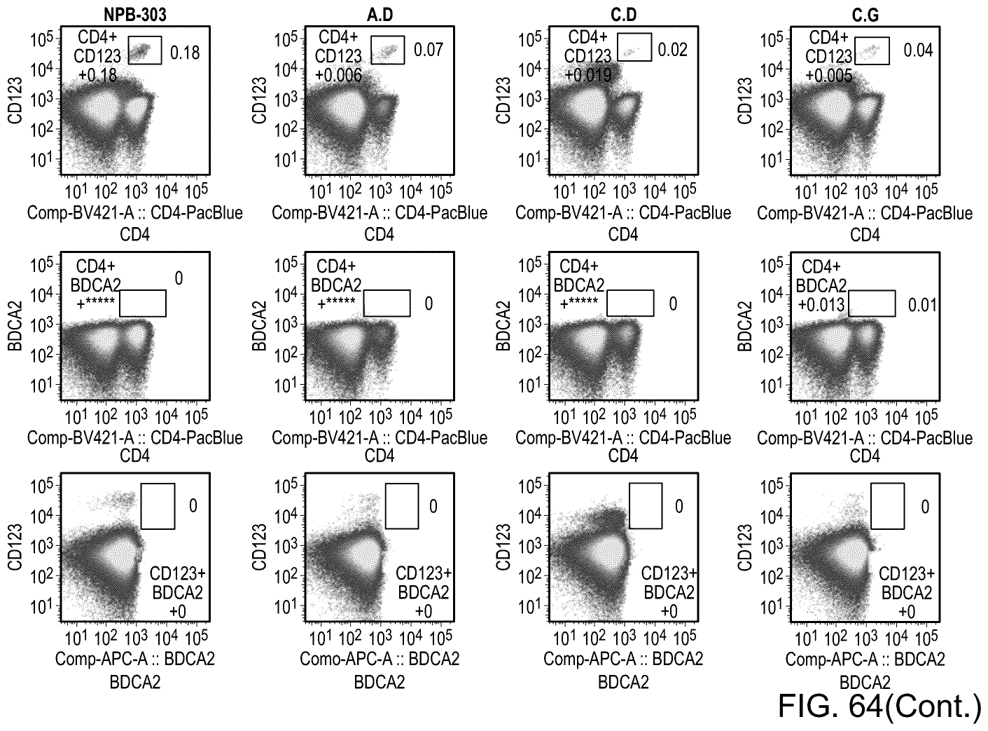

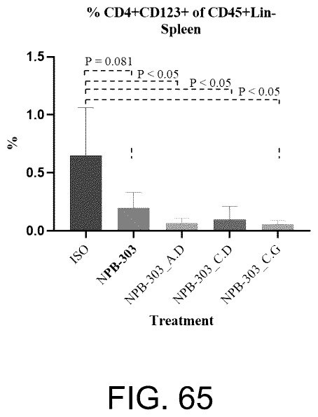

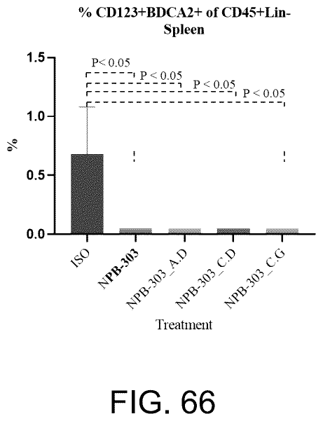

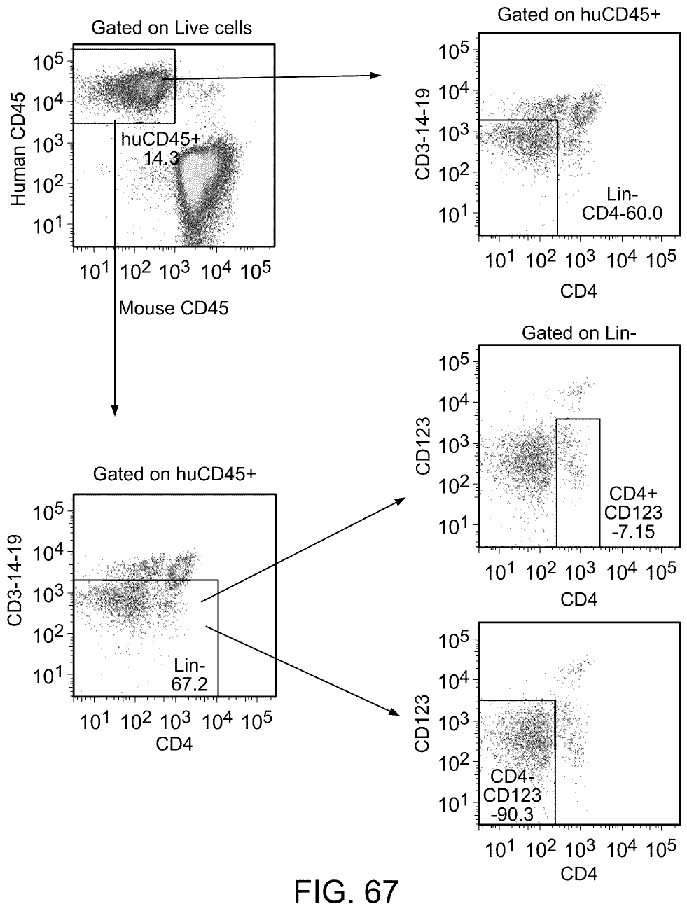

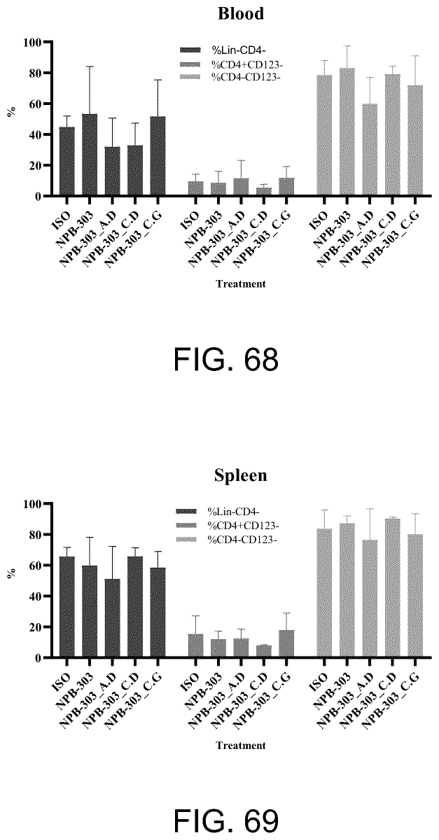

Representative Flow plots of blood samples for one mouse per groups were shown in FIG. 61. Human pDCs were depleted in all mice treated with NPB-303 antibodies compared to isotype control (FIG. 62), and BDCA2 expression was down-regulated in all mice treated with NPB-303 antibodies (FIG. 63). Splenocytes were also analyzed for pDC depletion by Flow Cytometry. Representative Flow plots of spleen from one mouse per group are shown in FIG. 64. Human pDC depletion (FIG. 65) and BDCA2 down-regulation (FIG. 66) were observed. In addition, other cell types including Lin−CD4−, CD4+CD123− and CD4−CD123− cells (FIG. 67) were not affected by NPB-303 antibody treatment in both blood (FIG. 68) and spleen (FIG. 69).

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. An antibody or fragment thereof comprising:
a heavy chain variable region comprising an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

2. The antibody or fragment thereof according to claim 1, wherein the antibody or fragment thereof binds an epitope having SEQ ID NO: 11 with an affinity (K_{d}) from 0.1 nanomolar (nM) to 100 nM.

3. The antibody or fragment thereof according to claim 1, wherein the antibody or fragment thereof is a monoclonal antibody or fragment thereof.

4. The antibody or fragment thereof according to claim 1, wherein the antibody or fragment thereof is a humanized antibody or fragment thereof.

5. The antibody or fragment thereof according to claim 1, wherein:
the heavy chain variable region comprises an amino acid sequence given by SEQ ID NO: 1 or a sequence at least about 90% identical to SEQ ID NO: 1; and
the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO:
10.

6. The antibody or fragment thereof according to claim 1, wherein:
the heavy chain variable region comprises an amino acid sequence given by SEQ ID NO: 2 or a sequence at least about 90% identical to SEQ ID NO: 2; and
the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO:
10.

7. The antibody or fragment thereof according to claim 1, wherein:
the heavy chain variable region comprises an amino acid sequence given by SEQ ID NO: 3 or a sequence at least about 90% identical to SEQ ID NO: 3; and
the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO:
10.

8. The antibody or fragment thereof according to claim 1, wherein:
the heavy chain variable region comprises an amino acid sequence given by SEQ ID NO: 4 or a sequence at least about 90% identical to SEQ ID NO: 4; and
the light chain variable region comprises an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO:
10.

9. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 5 or a sequence at least about 90% identical to SEQ ID NO: 5.

10. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 6 or a sequence at least about 90% identical to SEQ ID NO: 6.

11. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 7 or a sequence at least about 90% identical to SEQ ID NO: 7.

12. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 8 or a sequence at least about 90% identical to SEQ ID NO: 8.

13. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 9 or a sequence at least about 90% identical to SEQ ID NO: 9.

14. The antibody or fragment thereof according to claim 1, wherein:
the heavy chain variable region comprises an amino acid sequence selected from the group consisting of SEQ ID NO: 1, SEQ ID NO: 2, SEQ ID NO: 3, SEQ ID NO: 4, and a sequence at least about 90% identical to SEQ ID NO: 1, SEQ ID NO: 2, SEQ ID NO: 3, or SEQ ID NO: 4; and
the light chain variable region comprises an amino acid sequence given by SEQ ID NO: 10 or a sequence at least about 90% identical to SEQ ID NO: 10.

15. The antibody or fragment thereof according to claim 1, wherein:
the heavy chain variable region comprises an amino acid sequence given by SEQ ID NO: 1 or a sequence at least about 90% identical to SEQ ID NO: 1; and
the light chain variable region comprises an amino acid sequence given by SEQ ID NO: 5 or a sequence at least about 90% identical to SEQ ID NO: 5.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A method for depleting, inhibiting, or depleting and inhibiting plasmacytoid dendritic cells (pDC) in a patient in need thereof, the method comprising administering to the patient an effective amount of an antibody or fragment thereof that specifically binds to an epitope comprising blood dendritic cell antigen-2 (BDCA2) and depletes, inhibits, or depletes and inhibits pDC, thereby depleting, inhibiting, or depleting and inhibiting pDC; wherein the antibody or fragment thereof comprises:
a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and
a light chain variable region comprising an amino acid sequence selected from the group consisting of SEQ ID NO: 5, SEQ ID NO:6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

40. The method according to claim 39, wherein the epitope comprises an amino acid sequence given by SEQ ID NO: 11, and the antibody or fragment thereof binds the epitope with an affinity (K_{d}) from 0.1 nanomolar (nM) to 100 nM.

41. The method according to claim 39, wherein the antibody or fragment thereof is a monoclonal antibody or fragment thereof.

42. The method according to claim 39, wherein the antibody or fragment thereof is a humanized antibody or fragment thereof.

43. The method according to claim 39, wherein the BDCA2 is human BDCA2.

44. A method for treating a disorder associated with plasmacytoid dendritic cells (pDC) in a patient in need thereof, the method comprising administering to the patient an effective amount of an antibody or a fragment thereof that specifically binds to an epitope comprising blood dendritic cell antigen-2 (BDCA2) and depletes, inhibits, or depletes and inhibits pDC, thereby treating the disorder; wherein the antibody or fragment thereof comprises:
a heavy chain variable region comprising an amino acid sequence selected from the group consisting of SEQ ID NO: 1, SEQ ID NO:2, SEQ ID NO: 3, SEQ ID NO: 4, and a sequence at least about 90% identical to SEQ ID NO: 1, SEQ ID NO:2, SEQ ID NO: 3, or SEQ ID NO: 4; and
a light chain variable region comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, and a sequence at least about 90% identical to SEQ ID NO: 5, SEQ ID NO: 6, SEQ ID NO: 7, SEQ ID NO: 8, SEQ ID NO: 9, or SEQ ID NO: 10.

45. The method of claim 44, wherein the disorder is selected from the group consisting of an infectious disease, an autoimmune disease, an inflammatory condition, a respiratory condition, a skin disorder, a cancer, and a combination of two or more of these.

46. The method of claim 45, wherein the disorder is an infectious disease associated with infection by an infectious agent.

47. The method of claim 46, wherein the infectious agent is one or more viruses or one or more microorganisms.

48. The method of claim 47, wherein the infectious agent is a virus selected from the group consisting of Hepadnaviridae; Flaviviridae; Retroviridae; Herpesviridae; Papovaviridae; Rhabdoviridae; Paramyxoviridae; Reoviridae; Bunyaviridae; Filoviridae; Adenoviridae; Parvoviridae; Arenaviridae; Orthomyxoviridae; Poxviridae; Togaviridae; Coronaviridae; Picornaviridae; rhinoviruses; orbiviruses; picodnaviruses; encephalomyocarditis virus (EMV); Parainfluenza viruses; adenoviruses; Coxsackieviruses; Echoviruses; Rubeola virus; Rubella virus; human papillomaviruses; Canine distemper virus; Canine contagious hepatitis virus; Feline calicivirus; Feline rhinotracheitis virus; TGE virus (swine); Foot and mouth disease virus; simian virus 5; human parainfluenza virus type 2; human metapneuomovirus; and enteroviruses.

49. The method of claim 47, wherein the infectious agent is a microorganism selected from the group consisting of Rickettsia, Chlamydia, Mycobacteria, Clostridia, Corynebacteria, Mycoplasma, Ureaplasma, Legionella, Shi-gella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., and Helicobacter spp.

50. The method of claim 47, wherein the infectious agent is a microorganism selected from the group consisting of Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis (Yersinia pestis), Pseudomonas aeruginosa, Campylobacter jejuni, Clostridium dificile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

51. The method of claim 45, wherein the disorder comprises an autoimmune disease selected from the group consisting of diabetes, cutaneous psoriasis, hyperthyroidism, autoimmune adrenal insufficiency, hemolytic anemia, rheumatic carditis, systemic lupus erythematosus, cutaneous lupus erythematosis, psoriatic arthritis, Sjogren's syndrome polymyositis, myositis, multiple sclerosis, Crohn's disease, ulcerative colitis, lupus, inflammatory bowel syndrome, dermatomyositis, dermatitis, atopic dermatitis, scleroderma, polymyositis, psoriasis, alopecia areata, rheumatoid arthritis, graft-versus-host disease, interferonopathies, irritable bowel syndrome, and a combination of two or more of these.

52. The method of claim 45, wherein the disorder comprises an inflammatory condition, the inflammatory condition comprising acute kidney injury.

53. The method of claim 45, wherein the disorder comprises a respiratory condition, the respiratory condition comprising asthma, lung fibrosis, or a combination of asthma and lung fibrosis.

54. The method of claim 45, wherein the disorder comprises a skin disorder selected from the group consisting of scleroderma, psoriasis, atopic dermatitis, dermamyosititis, and a combination of two or more of these.

55. The method of claim 45, wherein the disorder comprises a cancer selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, B-cell acute lymphocytic leukemia, blastic plasmacytoid dendritic cell neoplasm (“BPDCN” or “pDC leukemia”), chronic lymphocytic leukemia, acute myelogenous leukemia, chronic myelogenous leukemia (“AML”), chronic granulocytic leukemia, acute granulocytic leukemia, aging-related hairy cell leukemia, chronic myelomonocytic leukemia (“CMML”), myelodysplastic syndrome, chronic myelofibrosis, multiple myeloma, neuroblastoma, rhabdomyosarcoma, Kaposi's sarcoma, Waldenström's macroglobulinemia, diffuse large B-cell lymphoma (DLBCL) (L265P), polycythemia vera, essential thrombocytosis, Hodgkin's disease, non-Hodgkin's lymphoma, soft-tissue sarcoma, osteogenic sarcoma, primary macroglobulinemia, retinoblastoma, and a combination of two or more of these.

56. The method of claim 44, wherein the epitope comprises an amino acid sequence given by SEQ ID NO: 11 and the antibody or fragment thereof binds the epitope with an affinity (K_{d}) from 0.1 nanomolar (nM) to 100 nM.

57. The method of claim 44, wherein the antibody or fragment thereof is a monoclonal antibody or fragment thereof.

58. The method of claim 44, wherein the antibody or fragment thereof is a humanized antibody or fragment thereof.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. A kit for depleting, inhibiting, or depleting and inhibiting plasmacytoid dendritic cells (pDC) in a patient in need thereof, said kit comprising:
instructions; and
the antibody or fragment thereof according to claim 1.

94. A pharmaceutical composition comprising:
at least one excipient; and
the antibody or fragment thereof according to claim 1.

95. (canceled)

96. (canceled)

97. (canceled)


SEQUENCE LISTING (14 sequences)

SEQ ID NO: 1 - protein, 118 aa, synthetic construct
  REGION 1..118: note = Heavy chain CDR3
EVQLVESGGGLVKPGGSLRLSCAASGFTFSTMSWVRQAPGKRLEWVASISSGGSTYYPDSVKGRFTISRDNAKNSLYLQMNSLRAEDTAVYYCARPSYDGYSSWFAYWGQGTLVTVSS

SEQ ID NO: 2 - protein, 118 aa, synthetic construct
  REGION 1..118: note = heavy chain CDR3 - modification A
EVQLVESGGGLVKPGGSLRLSCAASGFTFSTMSWVRQAPGKRLEWVASISSGGSTYYPDSVKGRFTISRDNAKNSLYLQMNSLRAEDTAVYYCARPSYEGYSSWFAYWGQGTLVTVSS

SEQ ID NO: 3 - protein, 118 aa, synthetic construct
  REGION 1..118: note = Heavy chain CDR3 - modification B
EVQLVESGGGLVKPGGSLRLSCAASGFTFSTMSWVRQAPGKRLEWVASISSGGSTYYPDSVKGRFTISRDNAKNSLYLQMNSLRAEDTAVYYCARPSYDAYSSWFAYWGQGTLVTVSS

SEQ ID NO: 4 - protein, 118 aa, synthetic construct
  REGION 1..118: note = Heavy chain CDR3 - modification C
EVQLVESGGGLVKPGGSLRLSCAASGFTFSTMSWVRQAPGKRLEWVASISSGGSTYYPDSVKGRFTISRDNAKNSLYLQMNSLRAEDTAVYYCARPSYSGYSSWFAYWGQGTLVTVSS

SEQ ID NO: 5 - protein, 111 aa, synthetic construct
  REGION 1..111: note = Light chain CDR1
EIVLTQSPATLSLSPGERATLSCKASQSVDYDGDGFMNWYQQKPGQAPRLLIYTASNLESGVPARFSGSGSGTDFTLTISSLEPEDFAVYYCQQSNEDPWTFGGGTKVEIK

SEQ ID NO: 6 - protein, 111 aa, synthetic construct
  REGION 1..111: note = Light chain CDR1 - modification D
EIVLTQSPATLSLSPGERATLSCKASQSVDYEGEGFMNWYQQKPGQAPRLLIYTASNLESGVPARFSGSGSGTDFTLTISSLEPEDFAVYYCQQSNEDPWTFGGGTKVEIK

SEQ ID NO: 7 - protein, 111 aa, synthetic construct
  REGION 1..111: note = Light chain CDR1 - modification E
EIVLTQSPATLSLSPGERATLSCKASQSVDYDADAFMNWYQQKPGQAPRLLIYTASNLESGVPARFSGSGSGTDFTLTISSLEPEDFAVYYCQQSNEDPWTFGGGTKVEIK

SEQ ID NO: 8 - protein, 111 aa, synthetic construct
  REGION 1..111: note = Light chain CDR1 - modification F
EIVLTQSPATLSLSPGERATLSCKASQSVDYEGDAFMNWYQQKPGQAPRLLIYTASNLESGVPARFSGSGSGTDFTLTISSLEPEDFAVYYCQQSNEDPWTFGGGTKVEIK

SEQ ID NO: 9 - protein, 111 aa, synthetic construct
  REGION 1..111: note = Light chain CDR1 - modification G
EIVLTQSPATLSLSPGERATLSCKASQSVDYDAEGFMNWYQQKPGQAPRLLIYTASNLESGVPARFSGSGSGTDFTLTISSLEPEDFAVYYCQQSNEDPWTFGGGTKVEIK

SEQ ID NO: 10 - protein, 111 aa, synthetic construct
  REGION 1..111: note = Light chain CDR1 - modification H
EIVLTQSPATLSLSPGERATLSCKASQSVDYSGSGFMNWYQQKPGQAPRLLIYTASNLESGVPARFSGSGSGTDFTLTISSLEPEDFAVYYCQQSNEDPWTFGGGTKVEIK

SEQ ID NO: 11 - protein, 22 aa, Homo sapiens
FISTGMQSWTKSQKNCSVMGAD

SEQ ID NO: 12 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 13 - protein, 448 aa, synthetic construct
  REGION 1..448: note = NPB-303 Heavy chain
EVQLVESGGGLVKPGGSLRLSCAASGFTFSTMSWVRQAPGKRLEWVASISSGGSTYYPDSVKGRFTISRDNAKNSLYLQMNSLRAEDTAVYYCARPSYDGYSSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 218 aa, synthetic construct
  REGION 1..218: note = NPB-303 Light chain
EIVLTQSPATLSLSPGERATLSCKASQSVDYDGDGFMNWYQQKPGQAPRLLIYTASNLESGVPARFSGSGSGTDFTLTISSLEPEDFAVYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC